SEQUENCE LISTING (5 sequences)

SEQ ID NO: 1 - DNA, 5341 bp, Canis lupus
  source 1..5341: mol_type = unassigned DNA; note = Canis familiaris
taactcaggaagtttaaaagtcatacaaaaattaggtggctgaaccagagtttgaacctagatgcttttatctactacacatacactgcttactttcccattaaataccacttcatcccaccccaaccaagtagtcctagggaaccacttcctggagcatagacatatcttcattcacctgtccttgagcaatgtctttatgtagaatgtgctgtttttctccattttctcattcttcaacattttgctcacactgcttatggaatattctgtccttatgcccttgttgaaattaatctattagagccacttatctgtcttcaataaaagtggttgacaagattgccttctctatcattgcaacacatgtaatttacctaaagaaatgaccttgccctaaaaaagtaatcataaatgaccttactctaacaaccgtcagatagtacaacaataaatttatttatacatggaaaagtacaactgataatgtatcaaagaagcttgaatatttaacaaatatttttaaacatgttttaaaacacatatgttttaaataaaatgaattaaatcatgttaaagatcaaatagaacttcgaacatgccaaaattctgatgagacaaatttgatgatgactcacttctgccctcaaagggcaggaggttggttgcatattgtggaatttcctctgacataattgaagaatgaggctgcatattctctgctgctctgaataatataaaatgccacagaagctctccatccaacagaagtcctttgggacagcagagctcacaagcgtctgggacaagagccagaaagaaaccagaacgaaggcaccttgtgtaaacatgacttccaagctggctgttgctctcttggcagcttttgtcctttctgcagctctctgtgaaggtaagcagacttttctggtttacagagttttcctgtgtctgaatgtgatcctcagatagcaagattacccttaatgctttaataacaaatatcctttttattcaagtaaaggaaataatatttgcagtcacttaacattaaattgaagctttgtttagattttatctctacaacacttagaaaagtataaagtttaattaatatagacatcttgttcctattttccgtgctctataggatgtttacatataaatgcattaatataaatctatagagcgtatctttttaaaaagatatatttatttatttttagagagtgagagaaaggtgggggggtagagacggagagagagagagagagagagagagagagagaatttcaagcagactcccagctgagtgtggagcccaatgtggggcccaatctaaccaccctgagatcatgacctgagcctaaatcaggtgtgagatgcttaaccaactgagcgccctagagtatatcttattagggccacaattactcatattatttattaatattgagcagataactcaggtaattttactacatgtactgttttattgacgaacttcagacaactatcatttcctgtttggtttaaagacaacaccccaacaaattgtaaatcttcttggaatccctcccatgaaagaaatacagttagaaatgtttatagcatagagttcttacatttttcttttttctgctaagcattaaagattttgctaagatctgagacatctttgtgcaatttctattgctattaattctttccaaaggcaatttttctgttactgaattttaggttatatcctataaatagcattgtggtatcatagaagattacacaatatcaagtagaaaaaaaagtaccataaggatgtcaacatttcacaaagtcaactccaagtcttatgcaggaagtcaatcaatgttgtcattaatgacagcatttttttttttaactgaatttaggggctaggagaatatccagccttctccttcaagaattagtttactaaagatgtgatgtttaccaaacatacagttaccaaattatcaacttggaattaaggagagaaagtaagtgaccagcagagtcatgtttgtgatgaaattaatcctagcttttattattttttcacagctgcagttctgtcaagagtcagttcagaacttcgatgccagtgtataaaaacacactccacacctttccatcccaaatatattaaagaactgagagtgattgacagtggcccacattgtgaaaactcagaaatcatgtaagtactttcaaaagatattttacttcggcaaatccataattgaggaaggtggaaatatccaacaaaattctaggtgtaggaatacaatagtgggcagaacagaaaggaaagattctttgtctctatgacatttaaatatggtaccttccattcaccatagttaaaaatgtcttggacccacattttatggttatactacaggaactacaacttgaagggcaaagggatgtctgaaacctgctaaatactgatttagtcatatctcttttatttcagtgtaaagcttttcaatggaaatgaggtgtgcctggaccccaaggaaaaatgggtacaaaaggttgtgcagatatttctaaagaagtaagttttttttttttttaactttagattcttcatttaccctgagacatatagtctaaatgttagcctataaatttcctcttgctgctggaagtctactcttttggaaatttgcctctttaattaaaaaacaaaaaaatagtagcaatagtgagtttgttgtactcataactgggaagaccatacatgtcagtttgtccagtgaagtctgggcttatgcctgtgccctgtatcagtgatatggttccctttcattctcagaagtgttctggtttagatgataatttatctttctactcataattgaaaattagagtatttctaacacatgaatatctgagattatttatttccatagtatatatattttcaagaatgatactttcctccttattagacatttaattaaaatttgttttattttatttattttcagggctgagaaacaagatccgtgaaacaacaaacacattctctgtggtttccaagaattcctcaggaaagatgccaatgagacttcaaaaaaatctatttcagtacttcatgtcccgtgtagacctggtgtaggattgccagataaaaatacagtatgcccagttagatttgaatattaagtaaaacaatgaatagtttttttctaaagtctcatatatgttgccctattcaatgtctaggcacacttacattaaacatattattcattgtttgctgtaaattcaaatgtagctggaaatcctggatatattttgttgttgttacatctttccacctcacctacaggccaggatgcatgagtcccttttcaaccttgccttggtctcttctttattcctcaactggagaaaaggtatcagcaagcatcctacctcacagaaatatgaggacatatggaagcactttaactttttctcatgttgtctaaattatgttcaagtgaaacttgtttgcctatttattatttatgtatttatttaagaaacaaatatgggaatatctgtgcataaatttggaaaaataggaaaggaagcattgttgataagttagtataatgatggtagtgaatttatatttattttggtatttagtgatgttatattaaagaactattttgttttttttttttttaaagaactattttgaacaaggttgctagatttagcaaaattaaaaatgagatactcatttaattttgatttcaaacaataattttttattatattattatttatctgaaatttcaattgaaccgcaatcctacttttgatactcctagtcttgtctattcactgacagccttgttcaatgctgggttgaatgatcagaaccctgagttagaattgtttctccaaagagcaaaaactcgacaagcaatattaatgaagtaatttcttgccagttaaaatttgtatatttataatatacaaatagattcttataattttacttattgtgttcttaaacactgacttttttgactttaagatgcttttatatgttccccaagagattttttttcctactatttttgatgctatggaaatagaaatgtaaaatatttaaaataaaacttattgtcaaagtcatcaggtgtttgtctttctttggtagttattgatttatttttttacattaaaaataatattttgagttgaaaagcatagatgcttggggttcctgggtggcttagtcagttaagtgtttgactcttggtttcagctcaggccacgatctcagaatcctgagacccagccctgcatagatttccacacttggcatggaggctgcttgggattctcactctcccttggtctctatcccctctcctgttctctctctgtctctctcaaataagtaaataaagtctaataaacaaacaaaaatgcttctctttgtgttttttatattatgtgggaaattataaacctttgtaaaaaaacaaatttttttcagagatgggttacaaacagtagtcctttccatagagtgatggaaattttctcaactccccaattgtattccattttgctaataagagtgacttacgattttcaattaaccaacccaggtatttgtacagcttagtacatctgagcactctgtgaaagagggaagctgatactcattataagccacttctcaactaattggggagtaaaactgacacctatgagataaggaattttaaatatgattaaagaccaagtatatttataaataagtcaaacaaaactcttaaaagtaaaaatgcaataatttgactaaaatactcagtggagaagttttataacagagtagacactgctgaaaaaaaaaaaaaatcagagatccaggaactaaaaccaaagaactttatctggagttcacatcagagagatatagatgtaatgaatagagaaaaacaatgagaagacatgggagatttagggataaggtcttactatatctagttggatttttccaggcagaggagagaggaaagggttaggaacatttgaagtgacaatggctaaaaacgtaccacgtgaagaagaataacaactcatagtttcaagaaatccaacttatcaatgcctaactttacatttctggtcaagaaagatggatcttagaactctgattcactttaatccacctcccagctcatttactaatgcttttagttattttaagtccactttcttttaaagctctacaaagttgagaggaagagagtcctctcctatttttctcactcattggttcatattgcaacctgctttt

SEQ ID NO: 2 - DNA, 33741 bp, Canis lupus
  source 1..33741: mol_type = unassigned DNA; note = Canis familiaris
atgctcaaccactgagccacccaggcgtccctatactgtttgtttgtttgtttgtttttaatttttatttatttatgatagtcacagagagagaaagagaggcagagacacaggcagagagaagcaggctccatgcaccgggagcccgacgtgggaatcgatcctgggtctccaggatcgcgccctaggccaaaggcaggtgccaaaccgctgcgccacccagggatccctgtttgtttgtttttaataaataggtgtaaaatgttttaaaatgagctggagtggtgtgtggtagattttgatgatttgtttttttcataatgagggaaaactaaccaagtagaatactagttatgtactctaatggacgacagtaccaggtaacaaaagttgtggcttttcttttaccactttctctaggttaagttttaatgtcagtaataatgaataaacctaaaactttatttgtcaacctctaaaacttaagtttaaaaagcgtatgtagtaagattcccagttttcataatatttttacataataaatgcttttttatgacattttatgatgtatataaatactttttatgacattaagtgaaatgactttccatcatacttaaaataaaacgtaaacattctttaccatggactgtaaaattttgtgtactcttgatttctgtctgccctccagcttcatctctttactctccccctcatgcttctatcacactggctttttgtcaggtcgtagattataccaagttctttcctgttacagtgcctctggctggcttttttacatggtgagatcctcactcctcaatcaagcccagctaaaatgtcccttccgactttccttgtgacctccctctctagaatagatctttcattcattatcaccaaatctattttctttttcacacatcacattcaactggttgacagatctttagctctactttaaggggattcctaccccttctctccatctctttaggccaggatctataacacaaaccttgctttgacacttgcattcacagcagccagatttcatgggaactttagagactattaaagatctacctgaagaaaaaaaaaaaaaaaaagatctacctgtactttggctcaaaggtctcctggaaaaaaatagacaataaagatatgaataagtccagtccatgaacaaataattatggtgttaggcatcaccatgatgaatgatcatttctgaacttaagaatacatctccataaagtagatcactattatttagtcttacaagtgtgggtacataagagcatgcattacctggtagagggacccagttcaactgaattttgaaaagaactctggtttctttctgtgccatgctaccttccattgaaatttgacttaaattttatgtgttgggttaggtcttgattcattcttattttaaggacaggattatataaagtgagtgagatcatttgcattagtctaatattcatttgttgtgctagatctccaacttgctgctgaacttgggaagacattactggatcggaacacagagttagaagattctcttcagcagatgtacacaaccaatcaggagcagttacaggaaattgaggtaatttactgattaagagagttacactttaagttgtggcatgttgctgctattgaccagtcatgttctgctaaaatagcactttcaaagaaccattggagagacttgggaagttgatttctatttttttgtgctttcccaacatatgtctgatgaaagaaatttctctctttaccttggtgaagtatactagaagcaaatggtccagtgtttaccattatgtgaaacacaataaaattgtgaatattatgcttcgtttttaactttttttaaatagattttatttatttatttgagagagagtgtgagccagagagagagagaaagagagagaaagtgagcacaagcaagtaagagcaggggtgagagagaaggagaagcaggctctctgctgagcaggaagcccgactcggggctcaatcctaggaccctggcgttgtgacccgctgaaggcagacacttaaccgactgaaccacccaggcacccctacttttttcttttaaaaaatgaattatgtaatatttcagatattcccagaggtgtaagagaatacaacgaatagctctgtacccaccacccagcttaagaaaaaactacccatactgttgaggctccctctttggttatacccccttccccaaccactgccaaataccatcctaagtttgggtttagatggaaaaaattttaaattgtagtactgatttcatgatgtgtttaaatttcttgtgccattcaaagaattaatcaggacaacccaggtggctcagcggtttagctctgccttcagcccagggtgtgatcctggagacccaggatcaattccctctcgggctccctgcatggagcctgcttctccctctgcctgtgtctctgcctctctctctctctctgtctctctcatgaataaataaataaaatcttaaaaaaaaaaaaaaaaagaatcagggatgcctggcaggccagtgggtagtgtatgcaactcttgatcttaagttcgagccctatgttgtgggtagtgtatacttaaaaaaaatcataaaatggatatttctttctctttgcttgatattaatttatttacatttttggtagatttttaaattttattttttttaaagattctattttattcatgagagagacagagaggcagagacaaaggcagagggagaagcaggctccccatggggaggccaatatgggactcgatcctgggacttctggatcacaccgtgagccaaaggcagacactcaatcactgagccacccaggcaaccctttagtagatattttttaaattaaaagagtacttttcccctaattataacaaactaaaatgagagttactagattaaggctttaaatgcaaggtgctggggtacctgggtggctcggttgttgagcgttggaacctcaatttcagctcaggccatgatataaggattgtgagattgagcccctagtaggctccttggtcaacatggagcctacttcagattctctctttccctcttcctctacccctccccacccccaccttacatatgtacatgcacacttgtgtgttctcaatctctctctttcaaataaaaaaataaaatctttttaaatattcaaaatgaaaaataaaatgcagggtgctatcttgtaaatggtcccattcaagtagtccagttatgggttggccatgtgactataaagatgataattttgggggtgcctcactggtgcaatcagtggagcttctaatttttgacttagggattgtgggtttaagccccttgttgggaaaaaaaaaaaaaaaagccccttgttgggtgtacagattacttaaaaataaaatctgtaagaaaatataaagatgataattttgaatattaacctgacagttgtctcattagaatacttttctctatgttccacctccccacatacctcccctacccaaaaaaaagctttgcacttaaatgacaactccttcctctcaaattggtacattagagacaggattcttgtgtttatgtgcacgaaaaatgtaaaagacctttgaacagtgatcagaacttactgagtggggtacttgggtggctcagttgtgactcttgatattggctcaagttatgatctcagggttgtgagatcaagccgtgcattgggctccacagtgggcattgcgtgtacctaagagtctctctcttccttgccctgtgcccctccctggacccccccccccaaagaaaatactaaaaaaggcaaattcataatctaaatatccttaattttacatagtagccataattaacgtgtgtatacagcagatctttccttcattaggatttatgaataatcaggtgactagctctgtgagaaatatgcttttataatctagaagggtatatcttgtaatgatattgaagttattagctaccacttttagcatgttctatatcaaaagagcatagaacatgccatacccagactaaatatagtttccaagaaacctaaaaatgcagaaaaacaagcattttaaaagctgttactaggagagcctggacgactcagtccattgggcatctgcctttggctcaggtcatgatcccagaatgctgggattgagccctatgtcaggccccttgctcagtggggagccaccttgtctctctccctctgcctgctgctccccctgcttactctctctgtcaaataaatatgtacaatcttaaaaaaaaaaaacaaaaaagcaaaactgttaaaactagccaagtgtcatacacaaagaattcatacttgctttgtgtcttagactttgctttacaactcagaagatttcatagagcagtcctagggccatatcatatgctgtgagacacatctatttctacttgggtagaaacgtagtctacttaggtagaaacatttaagagaatgatacctatttgttaccacaggagactatgaacacaattgctttatgatctaagtgacttattttcttctttcttctgccctgtggtattgaggatgattaaacagttaagacagattgttggcattgtgaaaaaaaatccacagatttttgaaattctctcaatcttcaaataacactataccatctattgtcttttataaggtgttgcaaagtgtaatggaaatgaagtgtccaattcagatcttaatgattaaataaatataaatttaaatacgtgtataacctattcaaataaatttagcctagaatattccggaaacattttcatgactcttctttccataataaagcatgtagttccctggttcatagatccaagcattgttgcattcaacattttactctttatccaaagctgtgttttggctgagaacaacactttcatagattttttttctctaccattgcagttggctatcttctttctttttataaatcactttatgaaagtcaatggggagggacaaaatgaggagatgaaagaaatggcagtgctggttaagctcacttagtagccagagcaccctccccccaaagtggccttattgcctgatcaggtcacatctatttctgtgcagacttacaagtcacaatattaactcccaatagtctaaaacaaggaaagcttgatgcttttataaaaggaagcaggcagccaaagagcctaattccgtactaaaaggtgaccgctttttatatgcttatcattgcaaccttcaggaaccacctaagctttatgaaggccctttccttaattcaaaccttatattagttctctctgtcttttaaaaagtaaactttttattgaaatacacatacagaggaatgcctgggtggctcagcagttgaacctctgcctttggcttaggtcgtggtcccatagtcctgggatcgggtcctgcgtcgggctccctgcatggagcctgcttctccctctgtctctgcatctctctctgtgtctctcatgaatgaataaataaaatgttaaagaaatatatatatatacagaaaaaagcacaaatcctaaatatatagttcaatgagtgttcattaacatactgatgaaaccaacacctaggtaagaaataagaacagtatcagctctgctagtcgtcacccacactaaccagtatgctaatacctagattaatttagcctgtttgaactttaccacttttacaggttttaaactaactttttagaatgtttattgggacttttcctgactagaaaagtaatatatgatcatttttaaagatagaaaaacatgtagttgaaaaccagaaagttagccttaccacctggaaaaatttgaaatgagatcatccagaagaatggtactgaaacttggaaaatactagtataaaatctcccaaactgtgacacattatagacattatagaaaaagacaaataaggaaactctcaagggtgttttataagaactacaagtgtgctttcaaagactcacagtggatttcaaataatcctcgtaaaaattgatcagtattttttagtgatcgttttaggtttctttttgttaaagaaaagagtccaagcatgagtaggtcaaggggcagagagagagaaccagactcccctatgagcagagagccctacacagggttccacctcaggaccctgagatcatgacctgagtaagccaaaggcagacgcttaacagactgagccacccaggtgtccctcagtgattgttttttatgtaaaaaaaaattaaactattgaaagtaaagattggcttcagagatacctatagtctgtgtgacttttattcagtgttgatttgaacatataatttttcaagactgccgagcaagtttttctctcctggatgaggggggaggagagaagccttggagggtaatgagatctggcaccatcagacataatcggattctcttaaagattcctataatcatgtaagcaagtctacagccttatgtttgtgttgcatctccgcttggaaacctgtattattcccataagccttgtcactaccactcaaagtagtctggccttcaagatatcctttcaagaacttatgtcttccttggaaattttaaggcattttattcctctgctacatttatcaatattctgtatatttatgatttgttctagtctacccacccatttcccagtgcaacattgaaggctgttgctatgtttattcatggtctttcacagagtaaacatcaataaatacttgttgaaacctatatccttttttttttttttttttttaagtaggctctgtgcccaacatggggcttgaactcatgaccctgagattgagagtcacatgctctaccaactgagccagccaggcaccccttaaatcagtattttagaaagggacagattagcaatttgcaaagcacttctgattcctaaactgtgggtttccaccttgagattttttttaaggcaataggaggggcactggctttagcattagaagattcaggtttaagccctccagcctatagcaaatagatggagactttaggcatgtgaattacctctcagatcctgttttttctgctgtataatggtgataagaatgcttgcactacattggcacagggttgtcctgaagggatgaggttatgtaagtgtattttggacagtataaattgtacaatttatccatgttataagaattttggtaatgaatcttaattcagaaaaaaatccttttcaattactggaaaattgattttaaaaacaagccaggcttttaatagattatattataggtggtttttatttttatattcatgcctcttctctccacccccccccccctttaaatttcctacaggggaaataaagtattttaatcagggaaaaggtttttcaaaggtaaaaggaaatatttcttcagaattgtagaagtcattttgatactaaactcagaggatttgggcaataatccttaatagaaggataatcccaacactgcataaaagccaaatttccattgcattccttatgtgttttaagttggggttttttgtcttttctcctttactccaggtcacggttctgcaattgcaaatagatagtaatcttgctggcctttctttttgaaagatttgcctcagttgttaagttgaactatttatagcttttgcttataacctgaaccttgtactcctagtcaaggtggcattctacgcaaattccaagaaacgtgtgtaataactcagagaactaaactttgatactcttggctataaaatcgacatttttgcctgtgtttttgctactgtcgtgtttgcctccccatgttttgcttaaatgtctccctttccgttgatatgtttaatatgacagatgcttttgtacttgagttttggcacttgaattttggatcttggcagtcctgtcgaattcatttgaggttgtgtgtcaaagattctgaatagagaaaaccagaacattagaagcttatctttgcctttaaatgaagggtagaaaatgagctaactcagtcccttctagattcagaaaaaaattgacaaatactaatttgaaactttggaggtcaagtgttaaagcaaggtctgaccacatacagttctgattctctggtctgataccactacttgctgggagatctaataatttttcttttaagttgctgaccaagttagtcagacttctttgttaggtaacctattacagattcttgtagaagtaaagggcttttttgattacttttgatttaataataataagattaaactcctaccacgtgaaaacaagctccctgccattctttagattgccattaaagccaagtcctctaagaccaaaatattacaagctcttcataaatggtttcattgtgtttttatcacaagtttatatatcaaactagaaggacttactatagtttttcttgatacataggtcctctccaccctcccagtctgtggttgcactttttgcagtttcagttacgtggtcagcccccatccagaaacagacaattctccttccgttgtatggtcagaaggtgccttggagcttcacactgtgtcacaatgcctgcgtcactcacctggcttatctcatcacgtgggtgttttatcagctcatgtcatcagaagaagtgtaggtacagcaaaggagatattttgagagagaccacattcacataacttttttttacagcctattgctataattctattttattattctcactgtgccagatttataaattagactttatcataggtgtatgtatagggaaaaaacatagtcatatatttagggtacggtgctatccactttttcaggcacctactgggagtcttggaatgtatccccagtggaaaagagggggactgctatcgcctctttgtactttatttacatatttgtttcctttatatttgaactttatatctttaaaaggggacaacaattaggggcccacataatagctggtaggaattataaatgtttttcagattactgggaataagtaatgtttttcagattgtgccatgggaataagtacagagaagtctataaatgaggcaaaagggagggaattagtggctattaggaacttttcccaagagggctataaccaaagcccttctcagaatcttttagttaagtctcctccacatttcagcagtcttagttgtggtaaagagcacgataagctgttgtgaatagaggtctagaaattgccattttggggcgcctagctggctcagttggtagagcatgtgactcttgttctcagggtcatgatagaaaggaattgccatttagccaatattttttgagtccctactatgtatcaggaattgtactaaatggactagaaagagaagaggcttcccttttcaaggagggtcagaaagactggacactgggatgccagggtggctcagcagttgagcgcctgcctttggcccaaggcgtgattctggagtcctgggatcgagtcccacattgggctgcccccatggagcctgcttctctctctgcctatatctctgcctctctctctgtcatgaataaataaaaatcttaaaaaaaaaaaaaaaaaaaagactggacactaacaagtaagcacttggtcatttgttattacgctaattgttttaaaggaaactgataagataccttgggaaaggtgctgcttgaggcagggtggtctggaaaggcttctgaaaagattatgtttgagcagaggccttaggacaatcagagttcttaaccattcttgtgccatagatccctgtggctgtctggtgaggtctgtgatgatcctcttaaaatatatagaataaaagggaatctgttatattgaaatgccttatcaaaatattaaaaaaataaatatgtgatatggtaatgtttgtcaacctaaaaagtataatttcaaatagtgatgagtgtagccaataaatcaagatttctgcaatcactatcatgtgatatgaaaatatctgtgatttctattggtgatgaagtcctagcaaccccatcatactacttatggcttgatggcttgttgcctccattcagaatcaaagcacgtggtaaatattggaagttagagaaaataaagatataacttctccctcattcaagttcacagatcctgtcagttctgtctgtggaccccagattaagaactgctgggttaaatggagccagttaggaaaaacacatcccctaacagaagggggagcttgcttagagtaaggcaactccatagttttaaggtaaagatactttgtgaaataaggatgaatcagtagctccctggtgaaactggaagagccacttaagagacagttgggaaaatcttaattaagactgaaaaataggggatccctgggtggcgccgtggtttggtgcctgcctttggcccagggcacgatcctggagacccgggatcaaatcccacgtcgggctcccggtgcatggagcctgcttctccctctgcctgtgtctctgcctctctctctctgtgtgactatcataaataaataaagaaaaaaaatatttaaaaaaaagactgaaaaataaatgatagtaaggggattattgggaggttttaggtatgattaaaattgtagttaaaacaaaaagtcttttagaaatatatgaatatggatatttaagatgtcttggatttgctttaaaatgattggagatgagaataggaaagtagaaatgtcacaagactgatgataattgttgaaactggatgatggatagatgggggcttattaaaccatttttttctcttttatatgtttgacattatccataatgttttaaaaacatagtagagggcaccccaggtggctcagctgtttagcgctgccttcgggccagggcctgatcctggggaccagggatcgagtcccacgttgggctccctatgtggagcctgcttctccctctgcctatgtctctgtctctctgtctgtctctctgtctctcctgaataaataaataaaatcttaaaaaaaaaaatgaaatctctaaaaacatagtagaggggcagccctggtggcttggcggtttagcgccgccttcagcccagggcccaatcctggagacctgggattgagtcccacgtcaggctccctgcatggagcctacttctccctctgcctgtgtctgcctctctgtctctgtctctctctctgcatctctcatgaagaaataaaatcttaaataaaaaaacacacacagtagataaaattagccatgtataaagagtctgtacttgggtggatgaaagaagcagcacacaaggtcaggaccactaatgtattatttcgggggatgtgactaaaagaacatattgaagcataactggtaaagaacgctgctggggtggttagaattccacttcatcttaatgaagagctgcaagtactagtcctatggagggaaatggtaaacatgaagcttaagaatcattgttgatccaagtgaatgctgatgcctcctggacctaagagaaaaagacattagtttggggggaggggagacttccagggttttccatgtccaagatcaacatttctctaataagtgtgatccatggacctcttatactaacctcacctggagtttttgctgaaaatgcagaatactgattgtaccctagtctcaccgactcaaaattttaggagaaaggggtataggttcctgtagtttgtttaataaagctctcagatagatgaatcttagcacatgcaaagtttgcaaactaactgtagctgcctcttcctgagcagttttaccatatgctgacgctgtgctgaacccttcacacacttaaaccttacggcagcctttgtgagataggtgatgaggaaatatcgcctgagaattaaggttacttgccaaaggtcacccggttagaggtggtgaagcagaacttcagattctaacctatgctcttagatactgctgcctcccaccaggctcttttctctgattctatacccaccaaaagatttctgtggaaattgacctctcacctgccctttacagaggatgtgggtcttccagtaactgggcagagacaattcctgtggtaaattagtaattagtgtctggtctatgtgttagcctttttcagtcgttgtaaatcagtgccaaagaatacttgacacctatacttgatataaaagtagcttgtgaagtttctgtaccctttgtatgtatacctgtatcctaaactttgtccctaagagattgaacacattcccatcagtcataggaacctactatgacatcgctagccagagggcaggattaggggccttctcccaggagaagctggtaggtactgaggcagtgaaggctaaacataggctttgagagggaccgtgatcagtgtgtgctcacctaccttatttaatatatttaaatcctacaagataggttagtcatcaattcataaccccttatttttcaattctgaacccaaaggctctataaaacaagatttttttttcctttaagtttgcagcagattcatttggcagaaaaccctgacctgaactgttgtgaagctgtttataatccttattcacacttaccattgcagaaatattaatgctcgggacacctgagcggctcagcagttgagcgtctgccttcggcctagggcgtgatcctggcgtctcagaattgagtcctgcatcgggctccctgcatggagcctgcttctctctctgcctatgtctctctgcctctctctctgtgtctcatgaataaataaaatcttaaaaaaaaaaaaaaaaaaagaaatgatcgattacaggatgctgctcataccacactgaggatgtttaaagtttttagtgtgtttgccacgttgtctttctacaattgaaagaattttcaaaaacatttttggcccctagagtttcagatgagaaagttgaagttatgagacttaagtgttttctcaacatattaccaagttatatcactagattttgaaaaagatcatttattctttttttttcaactgtcacattgtcatttatagtccactgaaaatcatttagcacattctttgaaagaagtcttttagctttgcaaatatagttgtacagatttacagttgtaaaggtgacttttgactcttgacaacatggatttgaactgtctgggtccacttacacaaggattttttttctgaaaatacagaacgtagtgtaaatatattttctcttcctcatgattttcttaacattttcttttctttagcttactatattgtgagaataagtatgtaattcgtataaacatacaaaatgtgttaatcaactgttcatgtcattagtaaggcttccagtcaatagtaggctattaaagtcttagaggaataaaaagttacaagtggatttttgactatgcggaagtttgtgccccaaccccatgttgatcaaggatcaactgtatagttgtacaaaaagccccaagaagtgttgtggggtttttgttttgttttgttttttaagattttatttatgagagagagagagacagaggcagacaaaggcagagggagaagcaggctccatgcagggagcctgacatgggacttgatccgggtctccaggatcaggccttgggccgaaggtggcgctaaaccgctgagccacctgggctgcccggttttgttttttctttttaagatttatttatttatttgagggagagcaagcaagcaagaggagcagaggagagagaataccaagccgactccaccctcagtgtggagcccagtgcagagcctgatctcatgtgcctgagatcacaacctcagctgaaaccaagagaccgatgcttaactgactgggccaccaggcacccaagacatgactttaaaaacattttccttggggcgcctaggtgacttagttaagcatctgcctttggctcagggtcatgatcctgggctcctggaatcaagccctgaattgggcactctgctcggggggggggggggggctgcttctccctctcctgtttcagcttggccaacttacgcttgttttttctgtgtcaaataagtaaaaattaaaaaaaataaaagagccactaaaaaaataagtaaataaaaatactattcttggggctcctgggtggctcagtcagttaagcatctgactcttgattttggttcaggttgtgatcccagggtcatgagattaggccccacatcaggtttcatattcagcagagtctgcttaagaccttctctctctctctgctcctactatgtgtacatgtgtgttctctctctcagaaaaatctttaaaaagtcatgtcttgggcacctggatggcttagtcaagtgtccaactcttgattgcagctcagatcttaatctcagggttgtgagttcagagcccacattgggctctgtgctataggctaggtaggcatgtagaatttaaaatcatgtctttggtcttttaccatttgatcattgatgatgctttgaacctacctttttttcctctttctagttgtgctccagagaaatgttaggaatatttcatttggaatatatactgtgtttcattgtcttctggtggagttgctgtaggagaggcagtcagtgtaatttaacactgaggcatgaagactgtgattattcccctttatccttggacatttagaaagtgattttagcatttaccagagtaaataatctggcaaatttctcatgaataaagaggattatggtggggcacctaggtggctcagtggttgagcatctgcctttggctcaggttgtgatcccagggttctgggatcgagttccgcatcaggctatctgcagggagcttgcttctctctgtctctgcctctctctgtctctcaggaataaaaaaaatctttaaaaaggattatgatgatcacattctcacaatattaaaaataacaagagtctttgaattatagtaagctaaatatctctttctggataagtcagttatttcccctgtatttttgtccagagtagctcatttggagaaaaaaattccatttttcttccaagcaaaaaatcatctaaaattctctacttcaggctttcttatttctgtaaaacatggtatttttgcttaaacattgatcatgtattccattgcttttactcttgtgggattgtcagtctcagatattcttgttgaatattaattttatgcatgcattgtgctttagaagagtagaccagcctcatgatacataggatgttggtaagcattaaggaaagtaaacatataaaggatggcctgtatttatttcctattagacaagtactttaattgggatttaaagattttatttattcatgaaagacagagaggcagagacatagagggagaagcaggctcctcacagggagtctgacgtaggacgtgatccctggaccctgtatcacgccctgaagtcaaccgctgagccacttaggtgtcccttaaagtttttctttacataaaatagacctgtggtattatggtataataaaaaccatgtatttgatctttctatttcgtggcacagagcttctaaaaccattggaatttcctgaggatagaatgggaactggttgccagaagaagcacaaccataatattagttagaaccatcagtccaacccccaacctctggggagggaatgggggttggagatggagttcagtcaccaatggccaatgatttcatcagtcgtgctatagaatgaagcttcaatgaaaccctgaaacaaggaacacattgaggtgctaataggctagtgcatcttaagagggtgtagaagctctacactcctcttcacccccatactttgctctgtgcatgtcttccatgtggctcttcctgagttgtctttttttttttttttttttttttttgagttgttatcctttttataaagtagtcagtatagggcagctcaggtggctcagtggtttagcgccaccttcggcccagggtgtgatcctgaagacctgggatcaagtcctgcatcgggctccctgcgtggggcctgcttctccctctgcctgtgcctctgcctctctgtgtgtgtttctcatgaataaataaataaaatcttaaaaaaaaaaagtcagtataagtaaagtgatctcctgaggtctgtgagttattggagtgaattattaaacctgaggggaggttgtggaaaccttcagatttacagctggttggtcagaagtacaggtggtctctggatctttcaactggtgtctgaagtgcagcaattttataggaccaagcccttctgacctggaatccctacaggtaggcagtgtcagaattgaattgtaagatacccagttggtgttggagaatcaagagtgggtgttcgaatgctgcgggtattttgtctgtttaatgtataacctggtcacaatcattttctcttacagtatctgaccaagcaggtggagcttctacggcagatgaatgaacagcatgcaaaggtttatgagcaattagatgtcacagcaagggaactggaagaaacaaatcaaaagctagttgctgacagcaaggcctcacagcaaaagattgtgaggtaaattttctagactccattgaggaagccctagagggagttacttagtgttgactagaatagaatgtggctttagtggatttgggtcaatatggggtacagtttaatttaattctttatggagaatagagattaaattagaagtcctttccaattaagagtgcccagtttgagtggctggcaactaatctctcctcactgctgatgcccttaatttaatatgacaagcagcaaacggcttgcttctgttcctcacagtctgactgaaacaatcgaatgtctacaaaccaacattgatcacctccagagccaggtggaggagttgaagtcatctggccaaagaaggaggagccagggaaaatgtgaccaggagaaaccggcacccagcttctcatgtctgaaggaactgtacgatctccgccagtaagaacctgctgttctgtggtattaatgagcggggaagcgtcgcctgtcagcgacacatccctgattcaagtagattaaaagtacccttttatgaattgctacttggtgattagactaccagtggtttgagcaaaaccacttaacttaaatatacaactgatcaagttcttgggcattagtaattctgcaaaaacttatttttagaaatcttgagaatattcatttcagtgctttgcatgtgttaagtcttcgtttaaattaagttgtacttctgatagactacaggtttgggggttgtatttcgcattagagtggaaaaaatcgaaatttcaaaaaaagcttttttgcctcagaaatgcctttatagggattcctgagtggctcagtggttgagcatctgcctttggctcagggtgtaatcccagggtctaggatcgagtcccacatcagactccttgcaggaagcctgcttctccctctgcctgtttctgcctctctctctctgtgtctcatgaataaataaataaaatctgggatccctgggtggcgcagcggtttggcgcctgcctttggcccagggcgcgatcctggagacccgggatcgaatcccacatcgggcttccggtgcatggagcctgcttctccctctgcctgtgtctctgcctctctctctctcactgtgtgcctatcataaataaataaattaaaaaaaaaaaaaataaataaataaataaataaataaataaaatcttttaagaaaaagaaatgcctttataatttagagttaagagactgaatatttaagaactacttcttgaggggatccctgggtggcacagcggtttggcgcctgcctttggcccagggcacgatcctggggacctgggatcgagtcccatgtcgggctcacggtgcatggagcctgcttctccctctgcctatgtctctgcctctctctctctctctctctctctgtgactattataaataaataaattaaaaattaaaaaaaaaaaaaagaacttcttgagcacatatgtggcaggtgctatgccagacatgaggagtggtgagcaaaatagacccagttcctgccctcccacttcagtctcggggagaggcagatacaaatcatcacacaaacacagttacaagttacctaggactttgcacaggagacctaggcttctgcatttggaggctctcaaccttgactcttgatcaagtcactttggaatttttcaaacagcaataccagtgagtccctcaggcctaaatcgggatctctgggtactgggaaaaacctgcccaggtgattctgatatgcagccaggcttgagaacccctgggccattctcatttccttcacaggagtcagtcatatcactggacttagaaaatgggtgtcctgttaatggttaaaaaaatagtcacaaaaattatgttttcttacatgtgcttatttgctagccttacagaatttcattttaaccattgctctagtgtagaataggcaaaaaattaatcactgtcacattcctcaaaaatattaaattgaaaggggatccctgggtggcgcagcggtttggcgcctgcctttggcccaggcgcgatcctggagacccgggatcgaatcccacgtcgggctcccggtgcatggagcctgcttctctttctgcctctctctctctctctctctctgtgagactatcataaatttaaaaaaatatatattaatttgaaaaaaaatcttttcattcccaattgacctatgtaacgtgtgtgtgtgtgtgtatttttttctttttggtaatatgttctttcaacatttttgtcctagacactttgtgtatgatcatgtgtttgctgagaagatcacttccttgcaaagtcagcaaagccctgatgaagaagaaaatgagcatttgaagaaaacagtgacgatgttacaggcccagctaagcctggagcggcaaaagcgggtgaccatggaggaggaatatgggcttgtgctcaaggagaacagtgagctggaacagcagctgggagccactgacgcctaccgagcgcgggcgctggaattggaggctgaggtggcagagatgcggcagatgctgcagtcagagcatccttttgtgaatggggttgagaagctggtgccagactctctattcatacctttcaaagaacacagccagagcctgctggaggagatgttcctgcctgtgtcggaagcacacagaaagcctctcaagcgcagcagcagtgagacagtgctaagcagcttggcagggggggacatcgtgaggggtcatgaggagacctgcattcggagggccaaggctgtgaagcagagaggcatctcccttctgcacgaagtggacactcagtatagcgccctgaaggtaaagtatgaggagctgctgaggaagtgccagcaggaagaggactccctgtcccataaggccgtgcagacctccagggcactggccaaagagacccagcctgagctcgacaccagcagttgggagccagcttctgctaccccagagcctgtcagttcccccaccaccaccacacctccagaatataaagcgctttttaaggagatctttagttgcatcaagaaaactaagcaagaaatagatgaacagagaacaaaataccgatctctttcctctcattcctaattcaacctccagctctactactaatttgcctatttcttttcacctctctctccaactccgacaagtgtttgtaaaccccgcagcctaatattactcatgatgtttgcctcgtagctttgcttatttagcagctgcgaacagcagcagggaaagaaggtggctgctggtgtctgttctataatccagatctgtttaaactcctcaggaaatcccatgacaactggcctctggctggcgcgctgattaggttgtgattccttgaaaagccccagaaccagtggagggatagatccatgctcctagggaagtaggcctggagttactatagtgtatagtatatagaggttgtatagtgtagaggagcaaaagggcagagttgaaacaaaattaaggcaacttttgctcctccttctcaaaaccatagaagtcagatggctgccaaactgaagtagctttcacaaacactgttggatactgtgaattcattaagaaagaatgtccaggagaaagcaggggtctaatccagaataaatggcattgacaaatactccaggccttgtgttttgttccatccactagtagttgagctgtgactgacaagtcataatgtgtctaagtccaaccaatgccgaactgaaaaggcaagctccggtttgaccacagttagcttgttagcagaggccctagagggccttccccaccccaaaccccccacctccagatggttttcctcaaagctgtagtggttagcagaggcagatgtgtaaatctttcatccctaaagtcaaaataaagcagcaagatctaagccctcactcctaccgtgttgtccgagctggctcagctgacagccacaagctacccttgttcagttcctgtcgttcctggaaatgtttgtgacttgtgcagaattcgtgtctgctaccttaattactaaacagtattattctgtgggaatctactcatcctctcacttgcatacttttaatttaaaactatttaagagaattgaaattccagttattgtatatatttttctaaaaaccaaataaaactacctatgaaaatgaacaaatggatcacttattttaagctgaagccatcttactgttataaccaacttcacattttttttattccaacagataactcagatttgaccatgattttttggagttattttttcccccattcttagggatcatgttaatgtgttcagtagaataactatatgattgaaaaatatgttcttaaagattttgtttatttgagagaaggagagagcatgagtggtagggagagggaggggaagaacagggagaggaagaagcagactccccacagagcagggagcccaacacagggctcagccccagtaccatgacctgagcagtaggcatatgctcaactgagccatacaggtgcccctatatcaccccatatttttaaacaagagtatactttaagcctttttcactatatctttcattttcactgaaccatgatctttgaaacatttatctgaccatctacatttttcttttttttaagatttatttgagaacaggaggaaccaggagggccagagggagagggaaaagtagactcctgctgagcagggagcccaacatggggctggattctgggcctccaggatcatgacctgagctgaatgcagatgcttaacccactgagctacctaggtgccccagaccatctgcattctttttttttttttttttaagattttatttattcatgagagatgcagagagagaagcaagctccatgcagggagcccaatgcaggactcaatccctggtctccaggatcacaacctgggccgaaggcggcgctaaaccactgagccacccgggctgccctgcattctttatgacagtactaaagtattacacctatggatataaactccatatgtatatttgccaatgagtgcagttgaggaaagaaggtggctgctgctgtcagttctataatccagttccatttaagctccaaaggaaatcccacgacaaactggcctctgtctggtgtgctgatgtgtgtatgtatgtaaatacatatattgtatatatagccatatataagaatccatttaagtagtccctgtatctttggacagttatgaatctagaaaataaatcattagaagtgaagtaaaaaagagcattttgccatcatagtctttcagaagttttctgattcccccagcttccaggccttgccaatacaggaggatgcctagttgctcacacctgtgccagtaaactttttgacccttgccaaacttatggaagaaaagtggagtttacttgtagtttagaattcgtttactaaagttgaatattttatagcccgttaaaccattcgtattgtattttgcctatttttcttatttaagagctcaagcattgaggaaattcattttgtaactagaagttacaaatactctatcttaattttaccttgttactggcattttgtgaagcaaaattttttgtggttatattagtttttcaatacttttttgtgctggacaggtgtttgatccacctaaagttcattttttttcttttattgtaaaaatacacatgacaattatcatcttaaccattttttaagtataaagtgttattaagtaaattcacattcttgtgcaactatctcagtcattcatccccatgactctcttcatttgtaaccatgaaacttggcccctttaacttctcattctcccttccccccagtttgcggcaactacctttaaacttttctgtctttatgattttgactaagtacctcacagaagtagaatctgtctttttgtgattggcttatttcactgagcataattacctcaaatttcatccatgtcgtagcatattgggtttcctttttaaggctgaatattattctatgtgtatatgccactttttgtttatccacccatcagtggacatttgggttgtttacatgttttacctattgtgactaacactgctatgaatgtgtgtacaaatatgcctttgagatcctgctttcagttctttgaatatatactcagaagaggaattactagatcaaatggtaattctgtttttatgttttgaggaaacttcatcatacggttttccacagtggctgtaccgttttacatgcctaccacagtacacattggttctagtttctccacatccttgccaaaatgtactttgtgattttttgatagtagccactctaaaggaagtggtatcttaacagttttgatttgcacttctctgatcagtgacacaactttcaatgtgcttggccatttgtatatctttcttggataaatgttcaaatcctttgccaatgtttgaatttgttttttctcttgagttacatgtatattcattctgggtattaatccgttatcagataatttacaaaaattttacatttaaatgttctaggggtttgcctttttacctttttttttttttttttttttgatgatttttatgtatttgacacagagggagagggagggaaagagcaagcacaggcagggggagttggcagagggagagaagtttccccactgatcagggagcccaatgcaggactcgatcccaagaccctgggaccatgacctgaactgaaggcagatgcctaactgactgaaccacccaggtgcctcgcctttttactctgttgagattgtcttttgatatataaggcttttaaattgtcatgaagtccagttaatctgttttttcttttgttacctgtgcctttggtgtcatgccaagagatcattgctgaacccagtattataaaacaaaatagtgttgctgaagggtccaacttcattctttagcatgtacataccgttttcctagcaccatttgctgaataggttgttctttctccattaatggtcttggaacccttatcaaaaatcatttgaccatatatgtgaggtttgtttctgggttctctgtattcctttggtctgtgtgtctcttgttatgccagtaccacactgttttgattacagtagctttgtagtaagtattgaaatcaggaagtatgattcctccagttatggtttttttttctccaagattgttttggctatttggtgtcccttgagtttccatatgaattttgggataattttatcatgtctgctaaaactgtcattgggatttagagatcgcatcaaatttgtagatcactgtgtaatattgacattttaacaatattaacccttctaatccatgaacatgaaatgtatttccactttgtcttaatttctttaagcaatgttttattttcattgtatgaatctttcacctctttggttgaattaattacaaggtatttcaaggtgcctggctggctcagtcagaggagcatgacttgatctcagagttgtgagttcaagccctacgttgggtgtagagcctaattaataaactaaaaataaataaataaatatttcattcttttggtgccattttaaaaggaattggcttttaatttcctttttgggctgttcattgttagtagtgtatagaaatgcaactaattgtggagtgttgactttgtattcaactactccgctgaattcatttattctgacaggttttttggtggaatctatagaattttatacatatatcatctgcaaataaaatttttataagatttattttggagagacagaacaagagaagaaaaggatggggtaaggagagagagagagagagagagagagagaaggaagcagactccctactgagcacgcagggagcctgatgcacctcaatctcatgaccctgagatgatctgcgcagaaatctagagtcatcttaacagactgagccacctaggtgcctctgcaagtagaaaattttacttcttccttcctaatctgagtaactttttatttatttttcttgcctaattgctttggctagaactcctttttttaaagatttatttattttttcatgagagagagagaggcagagacacaggaggaaggagaagcaggctccatgcagggagcctgttgcgggactcgatcctgggactccaggatcgcaccctgggccaaagacaggcactaaaccgctgagccacccagggatccccctggctagaactcaatactgttaaattggaagtggttaaagcaggcatctttgccttgttcctgaactttcggtaaatgctttcattctttcaatattgagtatgatgttcactgtgggtttttcatacatggcttttattatgttgaagtagttattttcctagttcattgagtgttttcttttttaaagattatttattcatagagacagagagagagagagagagagaggcagagacacaggcagagggagaagcaggctccatgctgagagcccgtcgcgggactcgatccagggtctccaggatcataccctgggctgcaggtggcgataaaccgctgcgccaccggggctgcccctgtttgtttgtttgtttgtttaaccatgaaaaaacgtagtattttgccatatgctttctctgcatcatttgagatgatcatgtgttgtgtttttttcttcattctgttgacgtggcatagtgtgttgataaatttttttaaacatgttgctattcattttttaaaattttttatttatgatagtcacagaaagagagagaggcagagacacagacagagaagcagtctccatgcaccaggagcttgacgtgggactcgatccggggtctccaggatcgcgccccgggccaaaggcaggcgccaaaccgctgcgccacccagggatcccctaaagtttctttagttgtaaaatcaggttgtagatttaatatctttattgtttttttaaagattttttaaaaagtaatcttgatacccaacatagggttcaaacacacaaccccaagatcaagagtcacatgctctaccaactgagccagacacatgtccttcttttttgtataaggtaagcatttataacactaagtttccccattagcactacttttgctgtgttccataagttttgggatgttgtatttccattttcattcatctctaagtattttctaagttgatccattggtagtttaaaactatattgtttgcagtttggtgaattttcctattttttgttatgatttctaaatatatcctattgtggtcaaggaagatattttgtatgatatcttaagatctgttgaaaatttgtggcctatggtctctccttaaaagtgtcccatacagagcagcctgggtggctcagtggtttggcactgccttcagcccagggcataatcctggagacccaggatcaagtcccatgttgggctccctacagggagcctgcttctccctccgcctgtgtctctgcctttctctctctctcaagaataaataaataaaatctttaaaaaaaaatggcctatacacacttgaaaagaatgtgtattgtgttgtgtaaagtgttctttatatgtctgctagatttagttggcttactgtgttaaatcctgtttccttagtcatcttctgtctggttgttctattattgtgaatggggtattaaaatctcctaatttccctggaatatcttttttccttcctttcaatttcaactggtttgtgtcattggatctcaacggagtctcttgtagacagcatctagttggattttgtgtgttttacaattttatttatttattcatgagagacacacagagaggcagagacacaggcagagggaaaagcaggctccctgcagggagcctgaagcaggactcaatcccaggaccctgggaccataccctgagctgaaggcagatgctcaaccactgagccacccaactgcctcatttgtgtgtattttattttattttattttatttaatatatttaaatattataaatattataaatattttttatttattcatgagagacagagagagaggcagagacacaggcagagggagaaacaggctccatgcagggagcctgacacaggactcgatctgggactccaggatcacaccctgggccaaagtcaggcactaaacctctgagccaaccagggatccccttgtgtgggttttaaattgaagtataattaacagagtgttagtttccaatgtacaatataatgattcagcagttctatacattgcttagtgctcaaggtaagtgtactcttaatccctattacctatttcaccattcctgcccccctcccctctggcaatcaccaatttgttttctgtattttagtctgatttgtttgtttcttaaattctacatatgagtgaaatcatgatatttgtctttctctagcttatttcacttagcataataccctcttagtccatccatgttggatcatgtgttttatttagtttgccaatctctgtcttttgattggagagtttaagccatttacatttaaagtaattttaataaggaggaaattctctcattgtgctatttgttctctgtatgcctcatagctttttttgtcctccatttcctgtattcttgccgtcttttatgtttattgattttttttttgtaatgagatgtttaaaactcctttctcattttcttttgtgtatatcctacagctattttcttcatggttagcatggggattacatttaacatcctagagttaaaacagtaatttgaatttatactttagcttaacttcaataacaagaaacaaaataaaaacaacctgttccatccccaccccttttggttattgttatcacaaagctacctcttgggatccctgggtggcgcagcggtttggcgcctgcctttggcccagggcgcgatcctggagactcgggatcgaatcccacgtcgggctcccggtgcatggagcctgcttcttcctctgcctgtgtctctgcctctctctctctctctgtgtgactatcataaataaataaaaattaaaaaaaaataaagctacctctttatgggtatataactgtacccaaaaacataaacttatatatttttttaatgcactggtcttttaagttatatagaagatcagatttgaagttacaaatcaaagttgcaggaggcacctgcgtagctcagtcagttcagcatcagactcttgattttagctgggttcatgatctcagggtcatgagactgagcccagtgctgggctctgtgctcatcttgaaggattctctctctccctctccctttgtccctctacccccaacactggtgctctcactcgctcattctctaaagtaaataaataaataaaatatttaaaaaacaaaacaaatcaaagttgcagtaatactacttttacactgataagttttctttaaatgtgtgtgtttgtttctttttaaagtaagctctatgcccaatgtggggcctgaactcacaaccccaagatcaagagtcacatgctctattgactgaggcagccaggcaatcccagatgcattagtctcttaaatcatgtagaaaacaaaaagtactattacaaaccattgttatagttatactggcttttataattgtccatacatttatttgcctttattgaaatttttgtttctcaatagggcttctggttaatatctagtgcccttttgtttcctgttgcaggacacatctaatggtcatgaactccctcagtttttgttacctgggaatgtcctaacttcttcacttttgaaggtcagttttactggataataaggttcttggttgacagtttttgttgttaccgttttcaaagcagtttgtatctattggcccactgtgttctggcctgtaatgttttaaatgagaaatcttttgattggggatccctgggtggctcggcagtttagcgcctgcctttggcccagggcacgatcctggagtcccgggatcaagtcccacatcgggctcccagcatggagcctgcttctccctcctcttgtgtctctgcttctctctctctatgtctatcataaataaataaataaatcttaaaaaaaaatcttttgattatcttattgaggatcccctgtgtatgaggatttgctcttctgcggcttttcaggattctctttggcttttgaaagtttaattataatgtctctcagtgggtctgagattatcttggagttacggagctgcttgaatgtttgtcttcatgccttttcatcaagtttgagcagttttcagccattatttcttcatgtattctctgcccctttctctctcctgctgggactcccacgtgtgtatattggtcttcttatggcgtcacacaggtcccttaggttttgctcactttcgtgttgtgtttctttgtttctctagaatttccatcatcttaccttcaagtttgctcattctttcttctgcttgctcaaatctgcctttgaatccctctagcgtatttttcacttccattgctgtgcttttcagctccagaattattttggtttgctctttaagtctgtcatttccattttactcacacatcattttctttatctacatcctctagttcttttcatgtctttttttttttttaatttatttatgatagtcacagagagagagagagagaggcagagacacaggcagagggagaagcaggctccatgcaccgggagcccgacatgggattcgatcccgagtctccaggatcgcgccctgggccaaaggcaggcgctaaaccgctgcgccacccagggatccccttttcatgtctttaaaacacatttgtcttaaagtcttggtctactgtatctgccattaggtctttcagggacagtttctgttgatttatttttttccttgccatgccctatattttcctggttttgtatgccttgtgattttttgaacaccagacattttaatttgatcatatagtaactctgggagtcagtctgatcctttcccaggttttgctggttttggttattggttttgttccttagattgttgcaggctgtgtctgtgctgaggatcagcctgaggcataaggttaaggttttctcaggtcttttgagactttctctgggcatgcatggtcactttctagtcttctctgtatatgtgatcatattttgaatatcctagttttcaatgtctggctccccaaagggagaaaagtgaaaaatgaaggggaggttgtggggaggatgccagtcctttgaataccttggcagtcacttgagcctcagctggagagacttgcagtgaagggggcaggtgtgtgcaacagtggctgcccacttctgcatctctggccagaagcagtaatccgtgcagattcctgatatttgaacagaatctttttggccacccaggttcctgcaagctgttgcaaactgctctgtgaatatggctagtggtggggttgttgctactctgctaggagccaaagttaaccaaaattgctggcaactgacggtcctgttcttcctggaagttataagcctttaggttctgtacagttctaaaatggttacatcagacaaattctgcttgtgcaattagtgtcctagagcaggagagatttctggtattctcacaccaccatctttctagaatcttcctaaacttcatctattccccatcacttgaaatcacctgaatcagggatccctgggtggctcagcagtttagcgcctgccttcagcccagggactccaggatcgagtcccacatcgggctccctgcatggagcctgcttctccctctgcctgggtctctgcctctctctcatgaataaataaataaataaaatctttaaaaaatcacctgaatcagtactaaatttctacatgaatttgaacctgtttctaagatctggttctcatgcacctgtggcaattacttactgtttagcttgaaattttcctattaagggaatattgctcccattattgctccttcagaatttcccccgtttttttggcgtgttttttcccccgatatgaactttaaagttagcttgtctgattcttggtgggaaatggttattgagatcaaatttaaaaattgattaaactggggatccctgggtgactcagcagtttagtgcctgcctgtggcccagtgcgtgatcctggagtcccaggatcgagtcccacatcgggctacctgcatggagcctgcttctcctcctgcctgtgtctctgcctctctctctctctctctgtctctcatgaatgaataatctttttttaaaaaaatggttaaattaagtatagactgtgacagactttggtccaaaaacatgggtttatgccttcccatttgttctagtggtctgtgtctcagtttccaaacttttatcacttaggtctacagattttagaaggttcacctcctggatgttataaggtcttttcttctgttaaaacatataaacgtgtgtgcatgtatgtatatatgttttatttaagtaatctctatacccaccatagggcttgaactcataaccctgagatcaagagttgaatgctcttccagctgagccagccaggcacctctcttgttgtgtgtgtgtgtatttaaagattttatttatttgagagtgcacacacaagcagggaggagggtcagagagggagaagcagactccccactgagcagggagcttgatgtggggctctgtcccaggaccctgagatcatgacctgagcctaatgaaggcagatgcttaaccaactgagccacccaggcacccattattttgtaactatagttacttataggaaagatactgactccagctgttttgttgaatggttgagttgcttttagttgacataactggagaaaaaaaaatctccacactcaacatggagcttgaactcacaaccctgattgagtcacatgctccaccaagccagcccgcccccgaacaagttttaacacctttttgtctaacttgttagaaggaaactaatttgtgggcagcccgggtggctcagtggtttagcaccgcctttagcccagggcatgatcctggagaccccagatcgagtcccacccacgtcaggctcccagcatggagcctgcttctccctctgcctctctctgtctctcatgaataaataaataaaatcttaaaaaaaaaaaaaaaaactaatttgtgtaaatttttaattttgatttaatcctttgtctaatttctctaagtagtatacccaggatattaattatgctgagcaaacatgtatctcaatcctaaggaatatgtggagggtttggttcagcaggttaaatttttaccgttttaacgaggttagtgagaattcatctattccacttttcaggacttatatttggaatgaattgtaggtctcgacccctgagaggagatcttttcccttctcatttcttattcatgttatgagctgcatggtagaagccctgataccacccttgcatttctgtgatcatagttattccatccccaggatcctcactttgttagacattttaggatttttttttttcaatgtgccgtttcaatctactacttacgattttaattcctatcatgctttgtcaggttttaaatcaattcttcagaagtatctggaggctcgttacatgctcaagtttaacccttgtttgtcatgtgatcatcattcatccgtgaaaatacgtgggctttttcttatttagtaactgatctattgtaatcttatttccaaccagttttaaggcttacgccattttccttcatcggctggtttatcttaacattaatttttatacctcatttttaacaatttcctatattcctgatttactttcctcatctgtttcaggctgtgactccccactgagccccataacctctctgtggtccactgcctctgctgtacaacatgaggggtctcccctttgtaagggggtcacacaatagctggacatactctccaactttggaattttttttcctcttttcaggctcatgtttactaacaattagtaccaagaacataatataccaccatcctatactataccaccaaatattagatgtttttctttttgttggtggggccaccacctaaaccttccggtagtagtggctctggtgtcccaaccagatcctggaagatcaggtagcagaatttcacatcttgcccaccccagcttactctgacttttgtcctaatcctctagggctgcactgtccactaaggtcaccattagctacatgtcccaactgagtgtttaaattgtggctggtcccaactgcaagtgtcggaagtatcaagtgccgcatcttgaaaacggtatcttacatttctttagtaaaaaaatgtcagtacagtttctttttactatttcaatgtgtctactagtaaacttaaaaattacatatgcgactgcaccgtactgccttctcccctcctccctcagctcccttcccggactacatgcgaggctctgggagcacagacaccactatcttcctggaaccctcagacgacagtagcaccatgatgccaggtcagtgttagtatttattttaaaaatacacaaaatataaatttttacatcaaagtgtaatgatctcacttatacattgttccatacttacctggttttgtttgcatccttatgcaaacattaatagatggatttgattctgattttttgctagggttcatgtaaacagctgagactgctacataaagtggttttaaaaggtaaggtggccacagaaacccaatggtgaaagaagaattcgatttggtttgtttaatgaaactggcagaagtcttagcacataggtaa

SEQ ID NO: 3 - DNA, 2328 bp, Canis lupus
  source 1..2328: mol_type = unassigned DNA; note = Canis familiaris
tacaagatttgggtaccctggtttggcgtttctcaatatttagaacatactcatataaaaaaaaaagattaattatctgtcacttagatttatctggattcttatatcttatctggcaaccctgctggtgtgaagccaaaaagttttgtctttgttgtcaaaactgcagcatcttacttaccaaaatgggcaaacagaacagagtagaacatcaacagaaaaatcccctccatcttatcgttctggattccatttttttcttggttttgatttccttgtggtttttgagcctcagagatttctcttactttcaatgggaagtctcaggattgggctttttggaaacaaaagaccaaaagtcttgcaggccacttctgcttcttgaattcccacataccactcccaaggcaaccagtacagacctactgacccagtagaacatggattgagggcaaaatatagaggaccaagatatgcattgctattttcaacatatgatccgcccacacagataaagggtttgcctagagctgttggttgtgtgaagtcattattgcggtaattgaaaaggactcgtttcttcttctcattgtgctgcagcaaaggcttcgtggcaagcccagcaggagccaagggacccaccccatggagagccgcggggtgatggaggcagccaatggctgcaaggatgccttggatttcaaccccatgaaggaatacatggtcctgaacagttcccaaaggatagctgttgcggtgctatgcacctccctcgccctgctaagtgccctggagaacctggccgtcctctacctggtcctgtcctcccaccggctccgcaggaagccctcatacctgttcattagcagcttggctgtggctgacttcctggccagtgtgatctttgcttgcaactttgtaaatttccacgtcttccatggcatggattccaaggctgttttcctactgaagattggcagcgtgaccatgaccttcacagcctctgtaggcagcctactgctgaccgccattgaccgctacctctgcctgtgccacccatctacatacaaagccctactcacctgtgggagggccctggcaaccctgggcatcatgtgggccctctctgcattggtctcctacttgccccttatgggatggacttgctgtcccaatccctgctctgagcttttccccctgatccccaatgactatttgctgggttggctcctgttcattgccttcctcttctttggcatcatctacacttatgggcatgtcctgtggaaggcccatcagcatgtagccagcttggctgaacaccaggacaggcaggtgccagggatggcacgaatgaggctagatgtgaggttggccaagaccctgggggtggtgctggctgtccttttcatatgctggctcccggtactggccctcatggtctacagcctgaccactaccctaagtgaccaggtcaagaaggtcttcgccttctgctccttgctctgtcttgtcaactccatgatcaaccccatcatttatgccctgcggagtggggagatccgctcctctgccttccattgcctggcccactggagaaggcatctgaggagacttgggcttgaaggaaacaaagaagtcccaaggtcctcagtcactgagacagaggctgatgtgaaaatcaccccatggccagattccagagtcctaaactgccctgattgctgatgacacctcttccacagttttaaaggagctgagtcagaatcatttcactcctcagaggaaagagagcagccttgaccctattgtcttacttgagtcaggctccaggggcttagacacttacccctttttgttgataactgatgtctctaacacctggcaggtagcctggctgtgcctatttgcatgagactgttggatagctagaccctgtgaggagtggcaaggtgggcgagaggattgtgcaggctcagtgcacattcagattacagaacctccccaacaaaatagcttagtgtttacatcttccagagaccccaggatccagagggagcctgcaggctcaccaaagaggaactcagaggaaatctagaaggtgaatggattatgttcctgatatctcaagatgtcaggtaggatagctggccaacaatgttcttgcttcattgttgggacatccttggttctaaggctatgaaagaccgcactttctgtcacctttgcctgctgaggaccaaaaactttgatacagtaaggattaagcatgttgattcacctctttcacaggtaattgacaagcctctagttcagggaatcttatta

SEQ ID NO: 4 - DNA, 7690 bp, Canis lupus
  source 1..7690: mol_type = unassigned DNA; note = Canis familiaris
cagttactgaatttcttcctctgtaaaatgggaaaaatgagaagaatagtaaatgagttgacatctgtaaaacctttagaatagtgcctggccccacagggtgagtgttctcaccttctttggaggctatatatgtcagagaaccctcctcttcccaaacagctttccatgtatgagagggaaaatacggatctgtaatgcatgagcaagaaaaactttgaaagcaaccaaataaacacctagaaatttctaatccatcctaactgccccccactatccccagggaagctgcatgctgacctctgatgaggacccctgccctaaaagaagcagctgaagatgccagcagagggagagattttccaccaccagtgccaatattagttcttctggagggcaaagaggaactgatgagctcactctggatcagaggcagtgagaaggagagagagaatgagactcaaatttccagaggctatttcagttttcttttccattttgtgcaatttcacttatgatatcaagcaatatgtggctgctgttttggaaactccccttgggggggcaccccctctactggcctgactctataaagggccagcctgagctgcagaagatcccttcagaggatcctgagacagcaggtggctctccatcagcaggtaccatgaaggtctccgcagctacctttgcaatcctcctcgccactgctaccttctgtgctcctgcctctgcctccccatgtaagtcaggtcctgatcactgcagcctggggccagaccctcacactggcccagactttgtcctcctttcaactgcagcatttaggcttcttgaacccgatgctttatattcctatatatatattcctatatattgctttacccagctgtccagctgtccagctgtccctgagagatctttaagccactccatggcagggaccctctgcttaacttttctgtcttcaagatctataccaagcccttcaggtgctgagcgcggactgatgctggggatatttgcaggcagaggcagcttgaactgacctcaaaaccctatcaccaggagggtttctcagaaacttctaagcttgggatagtcatattctccctgtttcatagatggaaaaagtgggccctgagagaggatttttttaattttaatttttttaagatttatttgagagagaatagaggggaggggttgagcgagaaggaaagaaagaatctccagcagactctcactgagcaaggagcactgatgcagggcttgatcccaccaccctgagatcatgacctgagctgaaatcaagagtgggatgcttaaccaactgtgccacccagaggattttttaaaaacctagtatggaccatgtattatgctgactgctctgcatgtaccatgtcatttaatacttcccatgactgagtggctgagcttgactgaaagacggagaaacagaggctcagaggagttaagtaacttgcctgaggtcacatagctaggaaatagggaaactggtgttcaaactcagtactgtctggctcctaaacactcctactttactgggaagtggcaactgacaccttccagggtcatcctgaattaactgaccaatagaaagctctccttccctgtttggaggccttcttaccatgcctcagccagagactcccaggacacaatacacatcttcaggcccctcttagcggttgaggctgagccatgaagactgaattttggaagcctccccccaacccactgtaggtctcaaagaaactaagttctgaatccactgaggatcacaagagggcaagaataaggacaaagagcccagacaagccaggaggaagcccatgatatccatagaaccaaaggcagaggattagggtgtagagggaaggatacatgcaaggctcaaggattagatagaagattgtggagatcttctccgtcctctgttggaatcacccactctgctactgtgtatccacagccaagtgactttacctctcggagcctcagtttcagccaaagaaggataacagtctatccaagataatgtacccagcacaataccccatgtataatggcaatgagtacaggagtctactgctttctcctcctagatgcctcagacaccacaccctgctgctttgcctacatttccggccgactacccttcacccacgtccaggagtatttctacaccagcagcaagtgctccatgccagcagtcgtgtgagtcccagccctgtggaccctctggggggggcagggggggatgaggaagggacaccttttgttccagagccagtcccccacaggagccctctaagagcctaccctatccactcccacccatcttccagctgctcaaatagccagagttcctccatccgtagcttcagatccccccctaacctttgtgtagaaggattaggcaggggttcctgggggatccttccccactctgatggagtggatcaggtctctgatctttatgggtgtacctacaatgtccctgcatggagacaagattgacatagccatctctctcaactaagaatcccatgttctcccatatgccacacccaatcaaatctacctgcaacctcagaggatctaagaaggagcagtcagaggtcagagagccctagttacagtcaaatgtactttaaaaaaaaattatcaactggtgaagaaataatgacatgagattctcctttaaatatgcaaactaacacctgaaaaattaacagtccttactacctgggatccctgggtggcgcagcggtttagcgcctgcctttggcccagggcgcgatcctggagactcgggatcgaatcccacgtcgggctcccggagcatggagcctgtttctccctctgcctgtgtctctgcctctctctctctctctctctctctcactctctctctgtgactatcataaataaataaataaaaaaatacctttaaaaaaaaaaaaaaacagtccttactacctagaagtgtgtatcaaatgccttacatggattatctccaaatttcatcacaacaaccctgacaggtatgtgtaattctctgcatgtttcagataatgaatctgaaacctagaggtttggagacttgcccaaagttgcacagtaagtgaaggatggagcctgtcttaagagtctcctctttctactaccgacagctcaacagtgacagttgcttaagaagtttaagataatagagacttaaagaatgaaaagggggtaatcgtgaagtttcctctataaagtgaagtttttttaatccaaaaattcctgtgacacttgcaaagccatatgcattcaagaattgcctttttttctggtgaatccttcaaagtgtcatggtaatatctagcactactaattttatttctgaaaacatatttttcaaaagaaaaaaacaatccttctttgagcacctgttatgggcaggtggagatgaaaaaaacactatccctgccctcatgggactcacagtctaatacaaggcagtgagatacagaatgattagagcagtcctgtgtaaaaaaacagtacagaagggcagcccccatggcgcagtggtttagcgctgcctgcagcctggggtgtgatcctggagatcagggatggagtcccacatcaggctccctgcatggagcctgcttctccctctgcctgtgtctctgcctctctctctctctctctctctctctctctctctctctgtgtgtctatgaataaatcaataaaatctttaaaaaaaatttttttaaataaaaaataaaaacagaaggagggagggagcacattcagccccggggattagtggatgtttgctgcaaaaggtgcttgagaaaagcctgagggacaaggtatgttaggcagactgagtgtcgcctggacgctcttttggttgatgcagcatccctgtgtagaaaagcagtggtgggcacttgagcatcagcacaaggactcaaaagtgttagagcaagtgaagtgaatgggcacagagaaagcctagagagccaggcccagtgaagtggctgttatggttacccatgtgagtagtattaaggctaaacaggacctgcaggaaagtagagggttggaaactgtcacgtatggagtcggcaagtcctggcaggtgaggggaggtcaggagcaaggacaagaagggagctaagtgaccttgaggtttctagccagagggctaagtgaccctgaggctggagaagtgtggcactccccgaagtctcaaggaaaaactgtttagaggataaagctaagatgccagcacgacaaccctgtggcaatgtccagcaggagttgttagccaggaagcagtgttgggcatagagataagacattcttttgtaggaggaaagagtctcctgagaggagagtagcaatctttagcagagtcttggtaagcgctggctcaagtgctcagcaagtcaggcttcattagaatcccagctctgccagttgggcagttgcaggagcctggacccatgagttaacgtctctgagcctccatttcttcagctgcaaagtggagtgaaatagaagtacttgcctcactgggtcaaagggagaataatgagatgaaaaacaaaattggaataggtcctggcacacagttggtacctgataagtgttcacttctcctcctccttctccaaaggtaatattatatacatagaagtgacaaaatttgatgagtgcaattcattcatgacctcatgctataaacatggggcattaagccccctcagtgtgccacacctagggccagaaactagggagaaaaacacagaaaggggcagcaattctgagaaacacctgcactcaaggggcaggtggaagtggttgctccggaaagaacaagcaggaagagagaggcaggagggaagtcaggcaaggagggggtccccaaaatgggggcagaaagaagcttagccagggaggttgttccccagtctcacattcttcaggaggaaatagaggcctgagaaaccctcaccccagatattcgtttcctgcccttttgatttcctggccacagacggaaggggccgagaagagaggataacaagtctgtaggatttcctgtctgatgtggctgaaccagggcagggtcattgtctaagttcccttactaccccaaataaacaaacactaggaggcatgagagtccagaatattccatctgctccctttctctcttctgcacattggaatggaagaaagagtgtcaagtggctctgagtctcagaaaaggggaagcagcttcctcaagagcaaacactatccagcatagaacccaaaaagaaaccaagaaatcctcatcctcgccagacgacctaagcttcactttgaaaatatttgagctcataaggtgcagaaaaacatcctaaagaagccaaaaggagaaaggtccaagtagacagggctggcccaaaagtaagatgtgaactgcgggcactgctctccagtccacagtctcccccaggtggaatcagtctcccagccaacaggtgaggtaggcagctggactaagcctgtgccccgcaggcttttatacatccaatgcatgccatcaactcctgtagagataaggcccttccctcccagtggcctccacattctagctggttaattggtagaagtgctttgacaccgggagtacctcggggctttcaccctctctgagcatggatgtccagtaccaggacctttggggtcatcttgctctgatccctcctaaagaagaggtcccataacattccaatgacaaaccacatgcggtgttttccaaaactccacaattccatctcatttgagtctcacaacaacagtgcaaaataggaagtatttctatttgtattttagaggtaaacaaatagattcagaggccctaggtgacttgccaaggtgaatccactaataagtgatgaaatcaagactcgggggtcatgtccttctatcattcacccacacacacatagatgtggttcgtttgttgagtgtctaccctttgccaggcactgtgttatctgaaggcaaatcgcatgtttgttgtaccacttcttctctttcacctactcaaccataactcagttcctagaagacccctctttttttctgcatcttggcacttgttaccaggtctaaaaccagaaatgctgagaaaaaactagtgattgaccagctcaatcctagactcctgagctccatatgcccttagctctccaggttgcgtacatatttctgtgggggttaatatgcacacagatctgcttctggcttggagccctacgattcaacagaagaggaaatatctttccttaaaagctacaaatccaactttgttggctccaagccaacagctccaagaatcctccactccagtaccatcatcatcatcatccccttgtgtctcagttcacttcacctccaaaaatggctgtccaggtgctgaccagatctcatcctgtcctcttcttccacagctttgtcacccgaaagcaccgccaggtgtgtgccaacccacagaagaaatgggtgcgggagtacatcaactctttggagatgagttaggatgtagggcatctggaacctgaactcatgcaaactttcctactgcttcttgctcttgtcctatgcagcttgggagaccctcaccaacccctacccccaccccttcctgggagggcacagatgccaccacccagtagcagttataaaagtagtagcagttataaataaaagcctcctccatctaaagtttgcaagagctccggaggctctgctttgtgcacagaaggtctctaggctcttgagtttcaggcctctcagcttgaccccagctctgcagtcaggaaggagatcaacaagcctccagagacgaagatgggaagaagactgggctccttcggcaatgtcccatggccacagctatcctctcagcccctcactgggaagggctgactgataaatgtgagaaaacagctttccattaaaattctcgctgcagtcaccaagctggctcctagtttgattttctcctcaggaaatttccttccttatgagcattaagtgattaacccatcttgaatttaactgacatgtacttttaatgctttaacagactttttagtgtgtctcttcaagtatttttttaggagttcagatcaaatctttgcagaatgttttctagtttggatttgcctggttgtttccccataattagattcaggttaagcatttctggcagggacactacatgagagatactgggtttctcagggcagcacatcagagcacatcggttcactgctggtgatgttgagtctgatggcccgaaggcccatgtttttttgccacagtgggctccccactgtgtgccaggcacaatgctaggtatgggaggctggtggggaaaaaagagagacgaatgagccacaagccaccgtctttaaagtttaggccaactgggatccctgggtggcgcagcggtttggcgcctgcctttggcccagggcgcgatcctggaaacccgggatcgagtctgtcgggctcccggtgcatggagcctgcttctccctctgcctgtgtctctgcctctctctctctgtgtgactatcataaataaataaaaattttt

SEQ ID NO: 5 - DNA, 26613 bp, Canis lupus
  source 1..26613: mol_type = unassigned DNA; note = Canis familiaris
tgtgtgcgcgcgcacgagtgtgcttggggggtgcgctggacgggcgcgtctccgcagaagtctgagcctgtctgtcggggtgcgcgtgtgcctgtccctgcctgtcgctgcttcttccgagtgctgggggccgcacaggtagcggcccccagggagcgccaagccccgggcgcggcgggggagctcgccagccccagccgcgggcctcgggggcggccgggcagcggcggggccccgcccgggccggccccgcgagcgggcgggagagccgagggccgggccgggccgggccgggccgggccgggcggggcgggcggggcgggcggggcggggcgctcgggccctcggggctgcccgcgcgccattggcggaaagttggcgcacgtcacgcggagggcagctccctaaagtcgtgcacataacgggcagcgcgcactccggggcggcttctcggggcgcaggctgtgggggccgcgcgggcgagcgcagagcaccccgcgagctggatgcggcttcccggcgcccgctcgctgcccgcggcgccgcccccgaggtccgcccgctgaggcgcccgtgcgctcacctgccggcccgcgcgccatgggccagcccgcgaaccgcagcgtcttcttgctggcgcccaacgggagccacgcgccggaccagggagactcgcaggagcggagcgaggcgtgggtggtgggcatgggcatcgtcatgtcgctcatcgtcctggccatcgtgttcgggaacgtgctggtcatcacggccatcgccaggttcgagcgtctgcagacggtcaccaactacttcatcacctccctggcctgtgctgacctggtcatgggcctggcggtggtgccctttggggccagccacatcctcatgaaaatgtggaccttcggcaacttctggtgtgagttttggacttccattgacgtattgtgcgtcacggccagcatcgagaccctgtgcgtgatcgcggtggaccgctactttgccatcacctcgcccttcaagtaccagagcctgctgaccaagaataaggcccgggtggtcattctgatggtgtggatcgtgtccggcctcacctccttcttgcccatccagatgcactggtaccgggccacccaccaggaagccatcaactgctacgccaaggagacgtgctgtgacttcttcacgaaccaagcctatgccattgcctcctccatcgtgtccttctacctacccctggtggtcatggtcttcgtctactccagggtcttccaggtggcccagaggcagctccagaagatcgacagatcggagggccgcttccatgcccaaaacctcagccaagtggagcaggatgggcggagcgggcacggacatcggaggtcctccaagttctgcttgaaggaacacaaggccctcaaaactctgggcatcatcatgggcactttcaccctgtgctggctgcccttcttcatcgtcaacatagtgcatgtgatccaggataacctcatccctaaggaagtttacatcctcctaaactgggtgggctacgtcaactctgctttcaatccccttatctactgccggagccctgacttcaggattgccttccaggagcttctgtgcctgcgcaggtcttccctgaaggcctatgggaatggctactccaacaacagtaacagcagaagcgactatgctggggagcacagtggatgtcacctggggcaggagaaagacagcgaactgctgtgtgaggaccccccaggcacggaagaccgtcaaggtactgtgcctagcgatagcgttgattcgcaggggaggaattgtagtacaaacgactcactgctgtaatgcagcttttctactttttatgaaccccctccccgcaacgaaacactatacagactatttaacttgagtgtaataaatttagaataaaattgtatagagatgtgcaggaggagggacggccttctgccttttttttttatttttttaagctgtaaaaaaaagagaaagcatattcgagtgattgtttgttgtacagttcagttccttttttgcatggaacgtgtaagtttgtgtctgaagggctttggtcccagaggacctggggctgctatgttttgatgacttttccgtggatctacctcattgatcaagtattaggggtaatatatattgctgctggtcatctgtatgtgaaggagtcttttcttcctgcacccttgcactggaggatcttgagtatctcggacctttcagctgtgaacacggactctgctggcccctcttatttgctcaaacagggtgttgttgtaggcagggatttgaggggcagcttcagttgtgttcctgagcaaagtctaaagtttacagtaaataaattgtttgaccatgacttcattgcacctgtttctccaaaaccccttgactggagtgtggtcgcctccccccactggaaaccgcaggtaactatttgtaataaataactgcccagggacttaatatggaatgatacaggaatggcacgcactgattgcttaaccctttcctttgcctttgactctgctgctgcgaacctgtatctctctcaatgccagtgcctcagagcagttgtgcctgccgtcagcatagcttgtctccctggtcctatggtgatgattctgtgttgttacatcagaaacactgactcacagaaacggagttacaggcatctgctttgtccctccacgcgcccccactgctcaccttctagtcctacttgtttaacagctgcttatgtcatatattggctgcagatctatatatagtggagatcttcgtactgtgtcaggtttggcattttaaagataaaggaagaagctcttatgcacaagctacccagacagagcccttgtcattccaagggaaaccgaaaagggcattggtcgtagggagaaagctggaaaacacttaaccgatgaataaatacctcatgacctcaaaacgagattgtaaaccttcctcctccttctgtctctcttcctcacttcgatataaagccagccaggcttctcagccttggcactgttaacgtctcgggccaggtaattttctgggcgggggcctgtcctgtgcatggtcagatgttcaacagcatctctggcctctctacccatcagatgccagcagcaccccctgaactgtgacaaccgaaactgtctccagaccttgccaaatactccgtgaggggtgaaatcacccctggttgagaaccacagatccaaggttccaaaatctaaacagggcagccgcttgattttgcagagcatccgagagggattttccatggttcactgggttagtgacagaccccgggccggcgctcttccctccacccctccaccctgccatgctgccttcccaactcaccttccagccgccccgacactgactgtatttgtggggatgtgcattgtttggtagggtttatccatggaggcgaggcctgtgagtctactgcctttttgtggagagattctagtaccgttaagaggcacatgagggaaaatcacaaaagtaaacacatttcttctcccaccccctttctatttttgcccgtgtgtctgagccagagcttggcccaggtttgaccgagtggaccgtcctccttggcaacggcagcctggagaggatcagcctgcaggttcattgccattgcacccgctcacgaagccgactccacttctctcttcccttctgtcccagccaaggtccccaaccagagaagctttggcttcctctgcttcctgtcagcgcagtgatgggtgtccgggtgcacatgcagctctcgggagaaagctaggtgcccgagttttcaggacgtgcagtggcaaattggagagtgtgtgtgtgcccttcagcgcttttactttttctccaagggctttgctgcctcttctcacattctcctcctgctctgggccccacacccagaggtgcgggcagcttctccagccagtcttcaccctcctggtggcctcacaggatcttggacttgaaagagcccatagaaagcatttggcgtccaaagagacgaaaaatctgtgagggggaatgacctgcccaaggtaagaagccaaggatgtcagagacggggctgcagccgtagaacccagctgtcagctgttattcctggaacagatgtcttccctccactgcatggccattgacttctgtcctcgccctccatggtggcagctttctttccctggggctgtcacagaacaaactcatgtcagtgggtgttactgctctcaggtccttagcgcagaattcaggcaatgaccaaataaccacaatagggataagagaaagaattgttcttttactcagcaagagtctactaggatatcctcagcgttggggaggcggggggacagggaggagcggggaagagatgcatgcttcctcgacccaccaggaattataagccactccaggtagaaatttcaaggagaaaaaaatgtaaacttttctgtcaccgttgtaagtcctgtggacaataaacgtgattaacaacaacaacaaaaaagttgtatctatattcttttggctagagaagcatctaatataatttcacttaaaatttgggtacaaggatgtttagccatattcgtgttcctttaggaatcctttaaatataaaaacttcgatgagttttaagggtcttgacaaaacagcattttacgtactgctcaatcccttgccttctgtcatcttttgttttgcgctttgagtggggggaggcgggtaggcatttgtcagtgtccctctgtccctctgtttgctgacaggttggttacataaggtttgtgttggattcatcatcctaaagttttctttgcctggacagcaagttctcagagtctgtcaagtgagaaccttatcctgagatgcaagctgacaggtgtgcaaagtggcaggccagggtgggcctccccactgaggtcactcttggggggaaggaatctggggaaaaaggagctgggaagtggggactgttgacaagcaatgcttagaatatttctgatgaaactactaccaaaagaaactcaggctggatcgatcagggccatttatgcagcccctactgtgtgcagcatgagaagagtatacacttctgtagcataagattaataatactaaagagatcccttttctggaatcttactaaatgatccagacagttctagacacctttttaggcattacatcatcttgatagccttatgatgtaggcaatataattgtcctctcttaacagatgtgacaacagaggcttggaaagattaagtaactttccaaagactcacaaaccagctagctgcagagtcaggactggaacccaggtctccgtgggtccaaacccatgcagaccaggaagcagcacaaggcagctcataatccatgccagaacgggcataagaactggaaacgcaatacctcggtgagagaagagggaggctgctatgggctggggcagggaggaatgtgcccttcgaagcatgtgactgcagggttgctggggactgggacaggaaaccaatcacccaagtgtctagtttgtttctctgagtctttgctttgtcttttcatcgagaattgggcaaaggtgaaaagaggatttcacacttgaaaacaagcatgggctagagcagaaatgcagcctgcctcacatcctgggaaatcatttttgcttctgaagttgggaagttctctgaaatggagcgggaacctagacccgatgggaagtgaggcagggctaggcggccctggaggtgcaccatccatttggagctatcgaattctagggtaagggagatagtctttggacagcagagagccatggatgcacttggaacgagacggcacggacagcgtaatgtccaataagttctgccctgctctaatccctccttcattgacagtgcaaatatcatttgaaggaggcatttttgaaatacctaagagaacaaacttcccaagcactctcaagggcttaagaaatactggtatcttatgaataataggaggcagggtgaaagaggccggcactgggaattcatgccgttaggttccaggtctccactctctctctctttctccctctctctttctctctctctctcatgttgggtcaggcagttctcctcagtggacctcatcttgcttgtctgtaaaatcacgaggccagtgcaggtggtctctaagctctgctcaggtatgaatttgtataaccattaatttagaagctaagtagagacactagtttgtaattctaattagaattgatgacatttgtcatataaataatgactgagtatcttgggggtggccagctggcagaggagaatgttgtgaagacctatttgatgcatgatacctgccttcaaatatttaaggaccatctggggaaaaggagcatatcaattttacatgaataccgaaaggcaagcagaagctcagaggaaggtcgattccccgctcaggtgaaaaaaaagactttaaaagtgaagtaggttatctatcagaatagtgagcttcctgtcattggaagcattcggagaagcccaaataaagctctgtgtggtgtgaagtacaaagggttcctgcacagagcaggagctggactaagctccttccaaatctgtgcctccatgtccttgaaaacaatagacctgaatgttcttaaaaagtattcagaccgttccctaattccagctggccttctccccaattcctctggatttcagaggtttcactgcattcctaaacaggaaacctccgggggaaatcaggatctcaacagtccttggccatggggggaaatccagaaatagagaaataaggttaagcagttttccaaactattagaccttttcatcagctgctcttcccatgccttttgcaaatgacctactaactcccagagagagaataatgagagctgcaaagtcctctctgcaaaggctattacatacttctccaccctcagaatcagtgcacttcctgtagtttttccgtcaccctcacagggcaggaagaaaaggaggcaggtccctggaaagcacggagcacttcaaagccctctggtccaagagcctagagacctggggtctctcacagcccagccactaaccagtagggccacctcggacaggtcactctccttctctagacctgttttcatcatctgtgatgagagatagtttggtttccatgaatcttgtgtttcctttttccagaattctgtacgatttcaggaagtatttgatgcggcacatctggccgcaatgcttttatttcaaaatcacatttttttttttctcattagaagggtaatagatgccctttgtagaacatttaaaaatatatgaaatcacaaaatgagataaatcacacataacattagcacctcgagagagccattgttatatgtgttgtatatgcttccagtctttgtcctgagcgtggcgatgcaatgcgtataggtatatttttaaacgaaaatgaattagattatgaatacaactttgatcccactgataataattttgcaaatgcctccttgaatatcttttatatataatataatccaggtatcataactggcctgacaatagaagcggccagcagttttcaaactttgggggtacagaggaccatcactcccagagcatctggattgggaaatctgagcaggggcccaagaatctgcattctagcaagttcccccaagtgtttctaacattctaaagttcccttgatccccaaggatcacattctgatgaataagtttatggagataaattctgtcatttatacagaatgtcccctctcttccttgctagttgacaaaaaggtgttgccatacttgtctctttgaaagcatgctttctgcaaatgggctctagttgctttcagagcataatcactaaactcctcgggtgaggttcttgctgcatcgttaaatgcctccaacccagccagttgatttatgttaaaataagtccattcaagttacatagtgaccaatacctactatatgaaaagtataacagcagatttctatccacagagcacctcttgggagaataatgactatgaactccagagggatactatgaacttggtatcatgcttggtggtttttgcgagtgatctcatttagtgcttaccctaaccctacagaatgggtacaattgtgtttctacccattttataaatagggaaactgagacttgaaggggttagctaagttgccaacaatgacgcagtccactggagtgaaacagagcccatgtgctttattgctaggatatgccgtcttccccccttgtggggtcagttaagagaaaaagaagaaaatgatgagaaggcatttttatttttatttcattttttaaaagattttatttttttattcatgagaggcacagggagagaggcagagacataggcagagggagaagcaggctccctacggggaccctgatgtgggaactccatcccaggatccaggatcatgacctgagccaaaggcagatgctcaaccactgagccacccaggtgtcctgagaaggcatttttaatgggcatgctttgttttattctccagggattctttcatgaggcctgattaatttctgagccttgcaactgagaggcaagcccctgccatcgagggtaggctgtggctaagtatgaacgaggacaccagattgccctgaaagcgggcagcaccctgtctttcaccatcattcaggcagtgtttctgagcttcccccaccaggtaggaagcagaggtgggccgcctggtaccagcacattggtttgcgctgagtcaaatcctgactgtcatggcaggaagcccgtgacatctggctttggggtgcttttcccatctctctgcccaggtctgtactcgtatctgaggtttcaccatagagaatatccagatggcatgggcggtgagcaagcccattacttcctaagtgtactaagaaagccaaggctctaagaggtggggtcggtgacaaggtagcccaggcaccacgagaggaccatctctcctggcttgttttattattatttttccagtgctgagaggggaagactgatgtttctgaatatattcacttcttcaggaagagaaaacagtgtttaaaatcacaacagcaatactcatgacaaagcaaaattagaggcctagggaactcttgccttctttcaactgtaactgcctttgtctggagagttttcttcattggacccattctgcctctgttctaccgtctagtgttcctctttatttatgaaaatcctattagatggtcttaaatactgcagacaaccctgataatctgttggtgggttttcattgttttcaatttggttgagaagtgcccacagtgcagagatgcattgccaactctaagcctgttctgctcacaacattcagatttctttcttgtttcacctctgtgtctctgtgattccaaacaatcacctggcctcctcaacctaacagaactgagggccagcctcaagggcctcaggtggccagccttagaaatctctttctataggatcgtggaagggttagtgatgtcagtataggtaggagtttcttggcgctattttattttactgcagtgggtcccaaaaaatgatttgggggaatttctgagaaaatgcatattcatgacccccgtctctgagactctgatctgacaggactctggttgggagccagcaatctgcattttgccacgacgactcccagaggactgcgacgcaggtgctctgaggcaattcgggaacctaatgctagcactgtcttcccttccccgtcctttccactgaagaacacaaaatgtaggcatacaaaagaaatgatcctgtacagttgcatacgttcacacacacacaaaaaatcatcctatcaaataaacaaataaataaatcgaatcctgactttcctgtgttgccaaataaaacaatgtagatgaaaacaatttgtatgtgataaagaattactctgactgacaataaaatgcctagtcccaaaccctcttcaatataccgggaggtagcagcacaggaagacaagggattttgctgcagtgatagactactactccagtgctccttcctccctgacaccaaaccttggttaaatcaaactatattaaagtctatatgaatctatttgcccaatgtacataaaccctcctacttagaaacctgatgggggtcccacccactacagagaagagtatgattcatacatattgacccatacacctctacacacagctgcacacacccaaataaacacacacacacacacacacacacagaattttatgttgatgcctatacctatatcaacataaatccacacaagcgcatttccacatagctatatggccacatagactcagacctaaaccttagagaaatacacacatagagattcctaagcatactttcgtgtgagcaaacatgccacgtcttgtaatcgtggtcccactgctacctacataggcacaagtccgtgtgcagaccccttttccttggtgagtctatatgtccttgattaaagcatgcgtactaaattcctattaagtgccaagaactgttagatgttgaagcacagtgagtgaataaaaacagttcatctgtgcccttatggagtttgtatgtgatgagagagaacttttaaaaaatgcatgccaagttgcaactgtcgttagtgctgcagaggagatttgtggtgttttgagggaatctgactaattctgagagttcggtaaaggctcacaaccccacaaagaagtgaggtttcaactaggagctgaaggatagctagataatctcttgaaaaagaaaggagataagagcatgccactcaggtagaaaagcttgtgcaaatgtcctggggtaagaggaaacctggtgcatgagacaaccgagcaaaggtcagtttagttggtgtgcagagagggagaatgaagcagggtatggggtaagcccgcaagaggcagacagatgccacatgtgaagggacttttaggccacattaaagatttgggtctttatcccaagggcttttagaagccactgatgtttacagtagggatcagatttgtatctactctacaaaagcactctcacgtagataaaggtgcacacaagggttcacacagacagttgggtgttgttttcttagaaatgcacgtttacatgtaaatattcatttaaaaacagagcctctctgaacacactcttatgtatttatgtgcatggtattgaaacccagcaagcatcagaggtgtggaaatatactcttcgctgagtatcatcaggcttaagccagtgtttgggggtgggggctagattagtgatgtatcagacctgatccaaaccctctaactttggagcttcgggctcttaggcttggcctcctgccttcacctcctgctccgctcccactcctcttggctcagagcacaccatgacaaggtggggtttcttccagaacatgatcattgactattcttggcctgggcaaggaggggaggatgggcaggtcaggttgcataaagattgccttgtgccctctcagatttgtaagcagagcctctgcccagcaccaagctcggagcaccctttgcacaaagttggtggtatcaagaagtaaatgtagcgatgggaaaccagaggtctgcaggtcctggtggggactgtggggggactccatgcagtgcattccataaggaaccactcactgcacgaaatgtgacactggaggaggaaacagacaaagccccgcaagaaagcctttcaaggcaagatgtcccacagtggccccactgccctggcacagtgaacacgtgtactcacatatttacacatcaacaaatcttgctctggtgggtgtcacatctccccacagagaaggtagcaagaaatttatcagccacaacagtctgcattctcttccttccgaagaactggaactacatttgtgtgtattttgctcctgtttaccagcaagccctccagggccttaaccagagccagtgacagactccagggaaatcagtaggaacattcatgccgaatgaaaggctcacctagagccttacgttctaaggccataaagatggtcccaagtgattttttttttatattttaaagactcctacccactgagattttttgttttccagtgtgtgcttagaaatgctttcttctataattaaaaagaattatgtaatgtaacttgaagctcttcttattttgcttcaggctgtgtaaggaacatccttcttttctcccagttgggtgctcttttatgctatcctggcagtgagctactagatccatcaagtgctgcagaccatagcactgtgctcaaggggacactgtggcataacaatccatattgatctagcacttggcagtttacaaagagcttttatatctgccatcttatctgatccttacaactctgaggtcagcatgcttgctcccatttcacagataagaagaccaaggttagaagcacccaggtggctctgtcagttaagtgtcctgtttttgattttggctcaggtcgtgatcccagggtcatgggatcaaggcctgcttcgtgctccgaactcagttgagtctgcttgtttttcttcctcagcttctgccccacttgctctctctctctctctctctctctctctctctctctcaaataaataaatagatctaaaaaaaaaaaaacaaagaaacactgagattaaaagatttgcctaagggcgtggagctaccacatggcagggttgggacttgaacttaggccttcatagttggtcaggaaataactcaagttgtcttgagctcaactgcccttctaggacttactcaggctcctctcactgttcttccctgctttcggcttgcattttatcttataaattcagtgctaacactccccagcaccgaagccacagctgcaaagctgcagataagtgactgcctgctccagagacagtccccgggatgttttccatattgacaagccccaggctagggtgagctcatgctgggaggagaccacgctcagctctgtcaactgtggcttagggcagaagctccaagccttttgggttcctcatgccagtcaccagatgcactttccccaagtgagagttaccggataacatacatgtccagctggagctcagggctctactgaggccttgcttgttcatttttcccaattcctcttgcatcaggatgtttctgtgggaagacccggtatgatttgccccaatcccacacccactgggcacagcgcaacctgcttctaagggccaagttccacaccttggtaaaaattgcaggctccaggaagtatttcagcttcttgaaaacagcttgtatttggcataaatgtcttattttagtcagagcaattagatgttcttgttttgcacacacttccaacagaattgccttcagcacaggtgaaaggtatttaatggttctgggaaagcttgagatagtggccacatcccaaccatgagaaacctccctgttggcaactgcttcctctcagagtgtcagcttatctgatttacttagctctccaaacaacatttgattgattgattgattgattcattcattcattcattcattcattcattcattccttgaaaagatgtttgaacacttataaatgccagagcctgtattagctacaagggtctatctagctatcttggtcacctaactatgaccaagataggtgtggctggctggtccccgtgttttagtttcttacagtctaatggagaagacagacaatgaaaagggaattataaatcatttgtggatcacattccttagtatgcacaccagagaaacttaaatatattgatgaaaaggggtgagagatgggcaattggcttcacaattttgctagctaggactaagaaatttgctgaaggatctgtcaacctcagactgactcctacccatccataaaaatattaatgtcttccctttaaaaacatctcaggggggcacctgggtgactcagtggtcatggtcccaaggtcctaggatcgagtcctgcgtcaagtttcccacagagagcctgcctcttcctctgcctatgtctctgcccctctgtgtttctcattaataaataaataaaatctttaaaaaaaaattttaaataaaaacatctcagggagggacacctgggtggggcagtgggttaagcatctgactcttggtttcagctcaggtggtgatctcagggtcatgagatagagctctgcatcagctctgtgctctgtgtggagtctgcttaggactctctctccctttgaccctctacctgcacactctctctacacgccccccaccccccatctcaaataaacaaaacttaaataaataaataaataaatgcacctcagggaaaagcatagttgacagtggcttccaaagttatcaatgatctctgtcttggtacatctctcgagtactgtgccaaattctgaggccacatttttcaagggaagggggacatttggggcccttccaggcttgccagatttagcaagtaaaaacataagacattcagttaaattttaatttcgaatcaccaacacattctttttttcatataaatatgtcccaaatactgcatggaacatacttataccaaaaaaaaaaaattttttttatcagaaattcaaacttaactaggcttcctgtgttttaatccaacagccctacattagagcaaagatagtgaagaaatctcaataatgtgtctcaaaaggaaacttaagaggagcctgactttttagcttggagacaatggtagtaagaacctagatttttgtaaagaagcattttccaaacattttccctaaaagcatatatggaaaatagtaaaaggtacactgagagggaaaaaaaatctttcgaggtcaaaagttggggaaacattgcatggccagtcttcccattttggagcttacactatacagaaacatggtaaacgttctgagtagacttgcaggaaacagacctggttgacctggtttagcttagcatgcctacagaatgccatttgtggtcttttgtctgtttgttttgatatgtttgctttatagaggacatctattaacattgtgcaaactggctactctgcccagaacaccttgggggacagtggcataaaaaaaatgaaagttcttttcaaatatccagagtgctcccgggtagggaggaaacagatttagtctttgtaaaccaaagggccgagctgtacccaatgcacggacacatctgggagacagattgctggcctggtataaagactagaactttctaacaattataatagtctgaaaggtaatgagctccctgtttggagaccttaaggcaaaaatgaaatgctatcagggacactgtaggaaattcttgttttgtgtgggaaattctctgcatctgctaggaagggtgtattcacataaaacacagagaggtctctaccagcaaacagactttcaaattatgtcccgtgttctatatcctttctgcagtctgagtctatccctcttgaatacattgttactgagttcccttcccatcactgggccaaacctctcaagaatattagtcctctcaagctaacgatctttggtctgatctgagcctcttcatagtctagcctaccttgcgtcttggctctttggaaccccaccttgagcctctgggagtgacttacagatttcatgagtaaactaaggcttacatctgagcttagtacctcggaagaggatggggtatggagaaacttggccctatgctcccccaggaccagtgagcttcccccgggtgggtgtctacagtgtggtagagaccaccgcatacctgaggtgctccacataacaaaaatcagtagagggtcaaatcccaaccccgcttcttcctgagtctatgacctggagaaaattgtttggcttctccaactttaatttccctctgtgaaaaaaaaaaaaaaaaaaaaaacaagaatgatgataactgcatcacaatatcatgaaaattaaaagagattaagagagtacattccaaaaaaaaaaaaaaagagtacattccaggcaaattgaacaccaataaaaaataaatttattattattaaaaaaaaaaagagtacagggatccctgggtggctcagcagtttggcgcctgcctttggcccagggcgtgatcctggagacccgggatcgaatcccacatcgggctcccggtgcatggagcctgcttctccctctgcctgtgtctctgcctctctctctttctctctctgtgactatcataaattaaaaaaaataataaaaaataaaaaaaataaaaaaaataaaaaaaagagtacattccaaagagccagcagagtgcgatacaaagtggacattaagtaaatgtttatttaatgtgtgactgggattgctactgttgtggtaaagactttcagagaagttaaaagatttctgtagaacttcaacgatcggtgtgccaagatttcaggccccagccaaaatctaatctagctcttgtctagagctctaacaccttttcagaacttgtgcttgagaatggaaagttatgcatgtgtattaaacatcacagttttccttcctgcgggtcttgggccagtgctaggaaaggcattggattcgggggcagattggtttaagtgactctgcttccttttccgtgctgaactctggtcctcatctgtaaaattaagaggttggactgagatttctctaggttccgtgagctgtgcctttgagctgggggcgatggatgcagatgtgtaagtaatggtccctggtccctaatgaaagaaagaccttgcccaactaaagcaatgtgtgtgtacatgtgtgtacacagcaacgcgatttgaagtcactttaattgccccctgctcttgtgcctgtctctaagtttgacaaagaaagagttgcaccagaattagctgtggaggagtgacctcctgatggggagagctgggttcgagtcccagtcctgactctgctggcagagcctgttccccatctccagggtgtgacactcccttccgcccccaccttggcctcccccatctttcacgtgagacaattggctccagggtctccaagctctgtgtctcctgggaaagcgtctctgatctattttcgcttggctttgcacgctctccgcaaggattctcatcgggatgactcaagttctgcttctttcttttgtttcatctgctctggtttcataacacaagctctccagccaattttagcatttcttggattatgggagtgtctacatgaaggtaccagagcactggtctccttttctgcccaggatgctggtctcccccttccccgccccgcccaccctgacactcccttccattctggcacaagcagactccctcctccccgctggatgacacactaagtggagtctgcttttcgcattagtcgagaggaggccctgaagtcagaaatccttggcacatgactcactttgtctctttgtgcctcctttcccccagtaataaataggtggagtctcacactttctcacctcccagtgtttgcccccccacccccagggaggtgtttttgtgcagagtgtcccctcttttcacagatgggctctctcgattatcatagccccagggcactctctaggaaaacagtatgtaccctccagacttcccttcattactgcccaagacacagcactttctaaaacacagaagtcaacttttgctgtaaagccttccatggctccctattaccttcaggataaagacctcagcagagcgagttcacaaggaagcaccccgaagatgaatgactcatctgcacatacagcccccctgacttgcactccttccctgggaactggaatgggccagggggtacagtgggaataaatagagaggggagcaggactcgggccatgtgtttaagcttatgtgccttccaaaaagggaatagacattcaaaaataataatggttttcttttttaagtactggaaatggtcttagaaatcactggcctggcccaactgcttcatttcacttgtggaaatggatgtcatagaattggcaaattattatttttttcaaagatgtattcattcatgagagacacacagggagagagagagagagaaagaggcagagacacaggcagagggagaagcaggccccacgcagggagcccgatgtgggactagatcccgggtctccaggatcacgccctgggctgaaggcaggtgctaaacctctgagccacccagggatcccagaattggcaaactataagagaccatgtctcttactaagtcgccgggtttctactctgcctctactaccaggggcccctctccactctgcagccagagtcatccttccaagggggatgggatgttgtcactccccatgtgaaagttttctgtggccccaatgcagctactgaagaagccaggcatcttcctggggccagggagccagtgtggtttgggtgcaaggtagacacacggccccactcactctccttctctttaccatcccttggtgcattcgcaggccagaccaccatctgcaccatcttgctgattcatttgggtttcttatttattatctgtgtccactcactagagggtaaactgcaggagggctggggttcgcctctaactctagtgtctacaacacagtggctcacagttggtacataataaatatttgctaaatgactaaccaaacaaacaaatgaatgcatattgaaggaaatttgaaaatggagaatagtataaagaaaccaataaaaaccactcatacttcctccttctagatattacatctgttaatattttggagtatttcttttttttcagatctttttttctccctaggtacatgtaggacatatatatatatatatatatattagatatataatatctaataggaaataaaattatattaaattgtggtttgtatcctgcttttttctctcatcattatgttgtgtttctacatctatggaattatcttttaaaataatattttaaatgctgcatagattacatacatatatcaaaattataaagcatttctctaattctggacactgacaatttttttttccctaagtgatctactttacctccaaaccattctatttttttaaaaaagattttatttatttgagagagagaaaagcagagaaagcatgagctgggggagctgcagagggagagggaaaaccaggctccccgctaaacagggagccctactggggctcaatccaggaccctaggatcgtgacttgagcagaaggcagaagcttaaccaactgagccacccagatgcccctctccaaactattctagtagaacttggcttttaaggtacttgcggtaactatttcttactttgagtagttgggtggaagacattactttctttccaggaaatgtcaacagatcaagcctttattttgtatgttttcccatcacctccttcagtggggattgacttagttgatgatctagttattaagtcaaagacttgtgtttagtacagggttgtgtttcaatgacttcctgttcgaaagataaagtactctaagtgttcacaactgagtagagtccatggagaaacggaggccaggccctgttacatctagtgcatactcttaactcaggggtgactgcctgttgaaattaatcaatgcttagggaccgaagcttcgagcgtttccaaatactgacttataaaatctctgtgaattaaaaggaaaagtaaagataactaacaccttcaagtgatgaaaaaggaaactgagactcaaaccgtatctttggtgaactagatttcagaaaagattttacagagctggaacggaccttcgagatgaactgtcccgaccttgtcatgttctaaagatgaggaaacagaggtccagacaaggaagggtgtcgtacccggtcaccagaccaccagcttgtggtggggctgcaatgagaagccacttcctcttccttggcacccatcagtctttccactgccctagctttgtggggccttgtcaccagccaacactcagcagcagaatctgcccttgggctctgtttcctggagtgctaagaaatctcctcacttcacacagaggattggaaagtcagagctttcttttttttttttttttttttttttaatattttgtgatgtcgaagatcacttttttttttaaatttttatttatttatgatagtcacagagagagagagagaggcagagacacaggcagagggagaagcaggctccatgcaccgggagcccaacataggattcgatcccgggtctccagaattgcgctctgggccaaaggcaggcgctaaaccactgcgccacccagggatcccggaaagtcagagctttcagggaagccctgctgtgagtgcagcaccacaggggtgccaggcataaaacaacacgtacaccctaacatccctttcttgtcagcccccacccccaccccaatccagctctatagcactttacgccctcagagtcccacggacctctccttgctcgctcttgccacactcctcttgcggcctgtggatggccagctctccctctagagtagcagcttcctgaaaccagaagctctttctctttctttgatttttcagcgctctgtagcaaatcattgcctagtccataatagatgttccgtaaatatctgctgaatacatgaataaaaacttagtttctgaaaaaaaaaataaaaaaaaaaaacccacttagtttctgtatcagctggctagggttgcataacaaaatatgttatgagtgccttaaacaacacacactcatttattcacagctgcagaggctggaagtccaagatcaaggagctggcagggttggcttcctctgaagccaccctccggctgcctcttcacagcatcctacgctctacacccgaacccctgccaccatctctatctgggtcctgatcccctcttctcagaaggaccccagccagattgcagcagggcccaccctaatgcctcacccgaactcagttccactcttcaaacggggggaatacagccaatttgtgaagtagtgggagttcacacttcaacatgtggatttggggagaaagcaaaaatgttgtcccagagggctggagagcatggagaggctgggaaggccaatcacaagtcagattaggagaggtcttattttatagaatagtaaaattgggctttattctctagacactgacaaacttttggttatttcagcacaaaatcatttcatcccacctcttcattttaccagcaaagaaaccaaggaccagagagaaaggacttgcccaaggccatgcagtaggtcaatggcagagctgacaccagctgacactgagatctctcaggcccgggtcagagctacactctgctaaattcggcctgcataagtgcatcgaaaccgacatcattttaaagaatgaggttttttttttttccccatgttttcccatgcctttctttttttttttttttttccccccatgcctttcatccagttttcatccactttcttttttgggcaattgtacagttccccaagtttcaagtaattaatacctttcatcaaatataactgatatggttcagtcagaaagatccccagaaatcactaatgatgctgttcccgtgtggctctgtggagtgtggccaaatggctcgctggtacattttacagactcgaaggacctgtatttcttacgtctaatagatagctcgagatagactgttacactaaacagctactctggccaagtttgttttaccacacatagacacacacacacgcacatgcacacgcacacatttggtacactatttcaggccgtttctgccatgcttagagctcctcatagcagtgattccccagctagaggatgtgtgtccaaccaggaaaatggaaaccactcagggatccctgggtggctcagcagtttagcaccagcctttggcccagggtgtgatcctggagacccaggatcgagtcccacgttgggctccctgcatggagcctgcttctccctctgcctgtgtctctgcctctctctctctgtgtctttcatgaataaataaataaaatcttaaaaaaaaaaaagaaaagaaaagaaaagaaaatggaaaccactctaggtctttctacttaagaaattcaatataaagaattggatgcacaagtgatagaagagctaagaagccaaacagggatggtaaggaatcagagattaacaacagcaggaggatgctgttgctcctgagcctagagagacacaggagaaggtggtgacacaagatcccagaagccaggggattatttggcaagagctggagccacagagcacatgaagccactgccagagatgccccttgagttagagaaagaaaggagaaacagtcagcttgtccccacctctcctccattcttctgtcatacactcccatcggccaaactgcagcatgtaggtccccatgtcaaaaagcagggcaggggaagcatgaggaaagggacagagagcaggcaccctggtggccttgggtaaatcatttgctctcagttttagttgccttgtggataatttgataatttctcacacgtttgttgggagaagcttcaaaggcacgtatataaatggaagggcttttattattggttttaattcaaagtatttgggactttgggctctgggggggacaaaagaggggtggtgatcaggtgtcatcaggagctttgtgatgcctccttaattgccctggtgaagggaagtaggtctgcattttaccacttgttattttattgtgtcactctgctccatcaggcttcaaaagccactgactgactttgaataaactggcacataactgaaaggatgttggagtgtgttctcggttctgtttggggagctctctgtggtttttgcatcaaaaagtgacacatagagaggactctagaaattactccaaagccattacctccaccccaccccttttgttttacagaggaccagagaggagacggggttggcctgttgtcccatgccaaattcttggcagagagagatgcacggcgctggaggagactcactgctttctccagtcctcctccttgggccttcgggtcttcagtcagccccatataacagctgatactgcaacatttccttgttctttgccatgatccttcttttccatgagcctcctctctttctttttcgtctttaacctcttcttcctagtagtctttgacaactcgttctcatggggttctttcgctccactctgctgatggacatcagaagcggagtgttgagccacatctattttaagttgagagtgtaaactcttctaaataactccattcccctttgatggcgggagtgagcaaggatcatcacaaactcttggaagaccagtttttctgccaacctcagggcttacggtgaaaataataaggtgccatctcttcctttctactggcagaaagtacagcccatggtcagtaagtggcaggccattccaaccaggcccatgagaacggtaggacttctaccaatggcacgaggtggtttagccctctcggcaagaatgggtctgtgttctgtatctttcagtgccagctaaccttgatgagtggatgataaagctcaaggatggattccttaacttgtcaaatgaagagattgtgccttgattgtaagttcccttccagcactgacatttaagaaatcagtatccttccaccaaagaagtcctattatggtcacagaggcactaagattgtaataatcccctgagtgactcatggaattcccaatggtccactgtgaattgacattttactggtgcagagtctgtggcatcttaaaatgtcagagctagaagagcatcagtcatcctctaccatgaggacattgaggagtctgggacctgaagcttgcaagtatccatttaccgattccttcagcagaaattgactatgcccgtgttatgtgccaagcagtgttctaggtcttagggtacagtaatagagaggagtagggatctgccctcacatatgcttgtagggtaagatgcaggacaactagggaactaaaaaaatgggtacaataattatagtttgtaataattgtttgaaaggcaataaaaatatattggattaattcactttgctcaatggatattgaataacttgggtcaggggaaagtgactgtttgtaacagtagcccttaagctgaagcgtaagaagaaaccagctgtggaaaccaggatgtaagaaagtaggcagagagaatgacatgtgcaaaggccttggggcaggaaagcttttggtatagccaagatgtggaaaggcagcgaatcaagtgggaatgtaataggtgaaaagtggctggaggagttggctgaaaccagattggctgcataggccctcataaggagtttaggttttattctgagattgggaagccattgaaaactcttaagcagttaagcaacatgttctgcttgcatttcttaaagactcctctgattgcagaaaggagaatggattgtagaagaggaaatgagactactgaaatagtccagacaaaaaagaaatgatagacaaaattagcagcaacggaaatggtaagaaatgggaggatttggaataacatagaggctctaagacttgtttcttacttggctacataaactgaggatcagaggaatcaaaaaagacttctaggctgttgagcaactgggtgaatggatgtttcactacagatgtggtaacaaacattaaaggtgggtaggattagagagatctatttactgtaaagggaaactggggttccaggggacacaaggttggggagtatacaggtatgagggtggcataagaattgcaatattagtgcctgggtggctcagtcggttaagtgtctaccttcagctcaggtcatgatctcagaatcctgagatcaagcttcacagtgggctctgctcagtgggaagtctgcttctccatctccctccacccctgcccccttcctgctcatcccctgcctcctccccactcatcctctctctctttctctctcaaataaatgaaagctttaaaataaaaaaaaaagaattgcaatattaccaaatatcatctatgccttaagagaaagtaagactatgaatgactttacagtggtgttattcagtgggtgtgatttggacccatttggcaatgtcctgagattcttttagttgttatgacttttggagagattgtagtttttctggcgtctaggggatagaggccagggatgctgctagatattcttcagtgtataaggcagcccatataacaaaaacttgtctggcctcacatgtcaacaatgctgaatttgagaactctatagattatttttggggttttatacaaatgaatcatgcccagagagcaatgtcttgaaaggtgtcactccagtgttaggaaggagtccagactgcttctgtggcaagaggctgatagtagctagtgaatatgagaccagcagtttccttgtggtggggctgggaccccaagagagggctctgagccctgtagggccttagtaagagcagaacattgattggatctgaccagagagcactaagtccagtgggacaccagatagatgcactgccaagtgagagctcacttattgagcaagaggaccaatggggatatgtctctgggggaaccaggggttgccagaggaagggaaagaggctgggagatcctaaggggccagagggtgggtagcaagtaccagttcccaaactagagatcctcaagagtctgctggttctcatgactgcctgcacagtaaaatcacctggggagattttaagagtattaatacctaggccccacccccaaagacagttattaagtttgtctggcttagggccctaacactggaattttaaagcccctccaggtattctgggttaagaaccactgatctaatccaagcattgcacacagaagcttccagaggcagacaaataaaggaagcaagggactgggaggccccatcgcagtggccagtctgtactcagctggagcccactatcaccctgtagagatctgggctcagtgttgccagaaatcttcccatttccttgttgttgtttttttcctctcaagaagcctaaaatccagaatttttttttttttttttttagtgtaagctccaaaaatcttaaaatcgcatgccaaacaaaacatgtgtgcggagtgctctggacctttgggctgtcatttagcaacctctgatccagtccagtggttttcacacttttttttagccacaggggttttttcctccaaacaagctattttgtgga

(19) United States
(12) Patent Application Publication
(10) Pub. No.: US 2024/0415803 A1
(43) Pub. Date: Dec. 19, 2024

(54) CANNABINOID COMPOSITION AND METHODS OF TREATMENT USING THE SAME

(71) Applicant: CANNPAL ANIMAL THERAPEUTICS LIMITED, New South Wales (AU)

(72) Inventors: Layton Patrick MILLS, New South Wales (AU); Margaret Ann CURTIS, New South Wales (AU); Rayson TAN, New South Wales (AU)

(21) Appl. No.: 18/819,337

(22) Filed: Aug. 29, 2024

Related U.S. Application Data
(63) Continuation of application No. 17/252,483, filed on Dec. 15, 2020, now Pat. No. 12,083,094.

(30) Foreign Application Priority Data
Jun. 15, 2018 (AU) 2018902143
May 3, 2019 (AU) 2019901525

(51) Int. Cl. A61K 31/352; A61K 9/00; A61K 31/05; A61K 47/14; A61P 25/04; G16H 20/13

(52) U.S. Cl. CPC A61K 31/352; A61K 9/0053; A61K 31/05; A61K 47/14; A61P 25/04; G16H 20/13

(57) ABSTRACT

The invention relates to a liquid composition comprising a combination of Δ⁹-tetrahydrocannabinol (THC) and cannabidiol (CBD) and an oral delivery system, methods of treating pain, inflammation and/or anxiety in a subject and methods of controlling a heart rate of a subject comprising orally administering the liquid composition, as well as downregulating or upregulating expression of particular related genes.

### CROSS-REFERENCE TO RELATED APPLICATIONS

This application is a continuation of U.S. patent application Ser. No. 17/252,483, filed Dec. 15, 2020, which is a US national phase of International Patent Application No. PCT/AU2019/050615, filed Jun. 14, 2019, which claims the benefit of the priority of Australian Patent Application No. 2018902143, filed Jun. 15, 2018, and Australian Patent Application No. 2019901525, filed May 3, 2019, the contents of which are incorporated by reference herein.

### SEQUENCE LISTING

The instant application contains a Sequence Listing which has been submitted electronically in XML file format and is hereby incorporated by reference in its entirety. Said XML copy, created on Aug. 19, 2024, is named 120755_000006_SL.xml and is 81,771 bytes in size.

### FIELD OF INVENTION

The invention relates to a composition comprising a combination of Δ⁹-tetrahydrocannabinol (THC) and cannabidiol (CBD). The invention also relates to methods using the composition.

### BACKGROUND

The following includes information that may be useful in understanding the present invention. It is not an admission that any of the information provided herein is prior art, or relevant, to the presently described or claimed inventions, or that any publication or document that is specifically or implicitly referenced is prior art.

There has been a resurgence in popularity of therapies targeting the endocannabinoid system, both due to increased scientific support for the efficacy of these treatments and relaxation of regulation of Cannabis products in some jurisdictions. This resurgence is also evidenced by the regulatory approval of some Cannabis-derived therapeutic compositions for a limited range of indications. The endocannabinoid system is highly conserved across many animal species, including mammals, such as companion animals. This system includes the cannabinoid receptors CB1, CB2, and GPR55, as well as the transient receptor potential ion channel TRPV1. The expression pattern for each of these proteins is different; however, the endocannabinoid system is expressed widely throughout the body, including in the central nervous system (CNS), the peripheral nervous system (PNS), gastrointestinal tract (GIT), immune and endocrine tissues, reproductive organs, and in the skeleton and/or tissues associated with skeletal functions.

Currently, medicinal Cannabis is popular among human patients and prescribers alike due to the reported benefits of cannabinoids, the active components of the Cannabis plant, and the long history of use which provides a known safety profile. This popularity has led many companies to develop formulations of cannabinoids for human patients. However, there may be significant interspecies differences in terms of formulation requirements.

Accordingly, there is a continuing need to develop cannabinoid-based treatment options that are suitable for administration to non-human subjects, preferably in addition to humans. There is also an ongoing need for alternative therapies for the treatment of pain, inflammation and/or anxiety.

### SUMMARY

In one aspect, the invention provides a liquid composition comprising a combination of Δ⁹-tetrahydrocannabinol (THC) and cannabidiol (CBD) and an oral delivery system.

In some embodiments, the ratio by weight of THC:CBD is 1:1.2 or more.

In some embodiments, the ratio by weight of THC:CBD is 1 or more:1.

In a further aspect, the invention provides a food product comprising the liquid composition of the invention.

In another aspect, the invention provides a method of treating pain, inflammation and/or anxiety, comprising orally administering to a subject in need thereof an effective amount of the liquid composition of the invention or the food product of the invention.

In a further aspect, the invention provides use of a liquid composition of the invention or the food product of the invention in the manufacture of a medicament for treating pain, inflammation and/or anxiety.

In another aspect, the invention provides a method of controlling a heart rate of a subject, comprising orally administering to the subject an effective amount of the liquid composition of the invention or the food product of the invention.

In a further aspect, the invention provides use of the liquid composition of the invention or the food product of the invention in the manufacture of a medicament for controlling a heart rate of a subject.

In another aspect, the invention provides a method of downregulating expression of chemokine (C-C motif) ligand 5 gene (CCL5, as shown in SEQ ID NO:4) and/or downregulating expression of cerebellar degeneration-related protein 2 gene (CDR2, as shown in SEQ ID NO:2) and/or upregulating expression of cannabinoid Receptor 2 gene (CNR2, as shown in SEQ ID NO:3) and/or upregulating expression of interleukin 8 gene (CXCL8, as shown in SEQ ID NO:1) and/or upregulating expression of adrenoreceptor beta 2 gene (ADRB2, as shown in SEQ ID NO:5), comprising contacting a cell with a combination of THC and CBD, wherein the ratio by weight of THC:CBD is 1 or more:1.

In a further aspect, the invention provides a method of downregulating expression of interleukin 8 gene (CXCL8, as shown in SEQ ID NO:1) and/or upregulating expression of chemokine (C-C motif) ligand 5 gene (CCL5, as shown in SEQ ID NO:4) and/or upregulating expression of cannabinoid Receptor 2 gene (CNR2, as shown in SEQ ID NO:3) and/or upregulating expression of adrenoreceptor beta 2 gene (ADRB2, as shown in SEQ ID NO:5), comprising contacting a cell with a combination of THC and CBD, wherein the ratio by weight of THC:CBD is 1:1.2 or more.

In another aspect, the invention provides a method of affecting the concentration of a biomarker associated with inflammation selected from granulocyte-macrophage colony-stimulating factor (GM-CSF) and interleukin-15 (IL-15) or a combination thereof, comprising contacting a cell with a combination of THC and CBD.

In a further aspect, the invention provides a kit comprising in separate parts:
(a) Δ⁹-tetrahydrocannabinol (THC) and an oral delivery system; and
(b) cannabidiol (CBD) and an oral delivery system.

### BRIEF DESCRIPTION OF THE ACCOMPANYING DRAWINGS

The invention may be further described with reference to the following non-limiting drawings, in which:

FIG. 1 shows a chart of the pharmacokinetic profile of THC in a first canine subject following administration of a composition of the invention;

FIG. 2 shows a chart of the pharmacokinetic profile of THC in a second canine subject following administration of a composition of the invention;

FIG. 3 shows a chart of the pharmacokinetic profile of THC in a third canine subject following administration of a composition of the invention;

FIG. 4 shows a chart of the pharmacokinetic profile of THC in a fourth canine subject following administration a composition of the first aspect of the invention;

FIG. 5 shows a chart of the pharmacokinetic profile of THC in a fifth canine subject following administration of a composition of the invention;

FIG. 6 shows a chart of the pharmacokinetic profile of THC in a sixth canine subject following administration of a composition of the invention;

FIG. 7 shows a chart of the pharmacokinetic profile of THC in a seventh canine subject following administration of a composition of the invention;

FIG. 8 shows a chart of the pharmacokinetic profile of THC in an eighth canine subject following administration of a composition of the invention;

FIG. 9 shows a chart of the pharmacokinetic profile of CBD in the first canine subject following administration of a composition of the invention;

FIG. 10 shows a chart of the pharmacokinetic profile of CBD in the second canine subject following administration of a composition of the invention;

FIG. 11 shows a chart of the pharmacokinetic profile of CBD in the third canine subject following administration of a composition of the invention;

FIG. 12 shows a chart of the pharmacokinetic profile of CBD in the fourth canine subject following administration of a composition of the invention;

FIG. 13 shows a chart of the pharmacokinetic profile of CBD in the fifth canine subject following administration of a composition of the invention;

FIG. 14 shows a chart of the pharmacokinetic profile of CBD in the sixth canine subject following administration of a composition of the invention;

FIG. 15 shows a chart of the pharmacokinetic profile of CBD in the seventh canine subject following administration of a composition of the invention;

FIG. 16 shows a chart of the pharmacokinetic profile of CBD in the eighth canine subject following administration of a composition of the invention;

FIG. 17 shows a plot of the heart rates of the 3 treatment groups (11 dogs) for the 12 hours post administration;

FIG. 18 shows a plot of the respiratory rates of the 3 treatment groups (11 dogs) for the 12 hours post administration;

FIG. 19 shows a plot of the heart rates of the 3 treatment groups (11 dogs) for the 72 hours post administration;

FIG. 20 shows a chart showing the change in chemokine ligand 5 gene (CCL5) expression at 1.5 h and 72 h in blood following administration of (i) medium-chain triglyceride (MCT) oil; (ii) a composition of the invention comprising a 2:1 ratio by weight of THC:CBD and MCT oil; and (iii) a composition of the invention comprising a 1:2 ratio by weight of THC:CBD and MCT oil;

FIG. 21 shows a chart showing the change in cerebellar degeneration-related protein 2 gene (CDR2) expression at 1.5 h and 72 h in blood following administration of (i) MCT oil; (ii) a composition of the invention comprising a 2:1 ratio by weight of THC:CBD and MCT oil; and (iii) a composition of the invention comprising a 1:2 ratio by weight of THC:BD and MCT oil;

FIG. 22 shows a chart showing the change in cannabinoid receptor 2 gene (CNR2) expression at 1.5 h and 72 h in blood following administration of (i) MCT oil; (ii) a composition of the invention comprising a 2:1 ratio by weight of THC:CBD and MCT oil; and (iii) a composition of the invention comprising a 1:2 ratio by weight of THC:CBD and MCT oil;

FIG. 23 shows a chart showing the change in interleukin 8 gene (CXCL8) expression at 1.5 h and 72 h in blood following administration of (i) MCT oil; (ii) a composition of the invention comprising a 2:1 ratio by weight of THC:CBD and MCT oil; and (iii) a composition of the invention comprising a 1:2 ratio by weight of THC:CBD and MCT oil;

FIG. 24 shows a chart showing the change in adrenoceptor beta 2 gene (ADRB2) expression at 1.5 h and 72 h in blood following administration of (i) MCT oil; (ii) a composition of the invention comprising a 2:1 ratio by weight of THC:CBD and MCT oil; and (iii) a composition of the invention comprising a 1:2 ratio by weight of THC:CBD and MCT oil;

FIG. 25 shows a chart of the concentration of granulocyte-macrophage colony-stimulating factor (GM-CSF) in blood over 24 hours following administration of (i) MCT oil; (ii) a composition of the invention comprising a 2:1 ratio by weight of THC:CBD and MCT oil; and (iii) a composition of the invention comprising a 1:2 ratio by weight of THC:CBD and MCT oil; and

FIG. 26 shows a chart of the concentration of interleukin 15 (IL-15) in blood over 24 hours following administration of (i) MCT oil; (ii) a composition of the invention comprising a 2:1 ratio by weight of THC:CBD and MCT oil; and (iii) a composition of the invention comprising a 1:2 ratio by weight of THC:CBD and MCT oil.

### DEFINITIONS

Before describing the present invention in detail, it is to be understood that this invention is not limited to particularly exemplified embodiments, such as the compositions, methods and uses, which may, of course, vary. It is also to be understood that the terminology used herein is for the purpose of describing particular embodiments of the invention only and is not intended to be limiting.

All publications, patents and patent applications that may be cited herein are hereby incorporated by reference in their entirety.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “administering” refers to providing the composition to a subject suffering from or at risk of the disorders(s) and/or condition(s) to be treated.

By “effective amount” it is meant an amount sufficient that, when administered to a subject, an amount of the composition is provided to achieve an effect. In the case of a therapeutic method, this effect may be the treatment of a pain, inflammation and/or anxiety, or to control a heart rate of a subject. Therefore, the “effective amount” may be a “therapeutically effective amount”. By “therapeutically effective amount” it is meant an amount sufficient that when administered to a subject an amount of composition is provided to treat the disease, disorder and/or condition, or a symptom of the disease, disorder and/or condition.

As used herein, the terms “treating”, “treatment”, “treat” and the like mean affecting a subject (e.g. a patient), tissue or cell to obtain a desired pharmacological and/or physiological effect. The effect may be prophylactic in terms of completely or partially preventing, or reducing the severity of, a disease or associated symptom, and/or may be therapeutic in terms of a partial or complete cure of a disease. For example, a reference to “treating” inflammation may therefore encompass: (a) arresting the progress of the disease, e.g. preventing worsening of a symptom or complication over time; (b) relieving or ameliorating the effects of inflammation, i.e. causing an improvement of at least one symptom or complication of inflammation; (c) preventing additional symptoms or complications of inflammation from developing; and/or (d) preventing inflammation or a symptom associated with inflammation from occurring in a subject. In another example, a reference to “treating” pain may therefore encompass: (a) preventing the severity of the pain from increasing; (b) relieving or ameliorating the severity of pain as experienced by the subject; (c) preventing the spread of pain from its originating location; and/or (d) preventing or delaying the onset of pain in the subject. In a further example, a reference to “treating” anxiety may therefore encompass: (a) preventing the severity of the anxiety from increasing, for example by preventing increase of a subject's heart rate; (b) relieving or ameliorating the severity of the anxiety as experienced by the subject in the short and/or long term, for example by lowering a heart rate of a subject; (c) preventing additional symptoms associated with anxiety from developing; and/or (d) preventing or delaying the onset of anxiety in the subject.

It must be noted that as used herein and in the appended claims, the singular forms “a,” “an,” and “the” include plural reference unless the context clearly dictates otherwise. Thus, for example, a reference to “a fatty acid” and/or “at least one fatty acid” may include one or more fatty acids, and so forth.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The term “(s)” following a noun contemplates the singular or plural form, or both.

The term “and/or” can mean “and” or “or”.

Unless the context requires otherwise, all percentages referred to herein are percentages by weight of the composition.

Unless the context requires otherwise, all amounts referred to herein are intended to be amounts by weight.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±25%, ±10%, ±5%, ±1% or ±0.1% of that value.

The term “comprising” is used in an inclusive sense, i.e. to specify the presence of the stated features but not to preclude the presence or addition of further features in various embodiments of the invention. When interpreting statements in this specification that include that term, the features, prefaced by that term in each statement, all need to be present but other features can also be present. Related terms such as “comprise” and “comprised” are to be interpreted in the same manner.

The term “pharmaceutically acceptable” in the context of a form of a compound or an additive to the composition, is intended to mean that the form of the compound or the additive to the composition is suitable for use in a pharmaceutical sense. Therefore, pharmaceutically acceptable forms and/or additives are non-toxic to the subject in the amounts in which they are present in the compositions described herein. In some embodiments, the composition of the invention is a nutraceutical composition. It will be appreciated that any ingredient that is pharmaceutically acceptable will also be suitable for nutraceutical use.

The term “veterinary acceptable” in the context of a form of a compound or an additive to the composition, is intended to mean that the form of the compound or the additive to the composition is suitable for use in a veterinary sense. Therefore, veterinary acceptable forms and/or additives are non-toxic to the non-human subject in the amounts in which they are present in the composition described herein.

The term “nutraceutically acceptable” in the context of a form of a compound or an additive to the composition, is intended to mean that the form of the compound of the additive to the composition is suitable for use in a nutraceutical sense. Therefore, nutraceutically acceptable forms and/or additives are non-toxic to the subject in the amounts in which they are present in the composition described herein. It will be appreciated that all pharmaceutically acceptable forms and additives will typically also be nutraceutically acceptable.

The term “cannabinoid” as used herein relates to any compound that has activity involving the endocannabinoid system.

The term “phytocannabinoid” refers to a cannabinoid that has been reported in an extract of a Cannabis plant, whether derived from a Cannabis plant or synthetically created.

The term “cannabinoid fraction” is used to describe the combination of cannabinoids present in the Cannabis extract.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### DESCRIPTION OF EMBODIMENT(S)

The invention provides a liquid composition comprising a combination of Δ⁹-tetrahydrocannabinol (THC) and cannabidiol (CBD) and an oral delivery system.

The inventors have surprisingly found that oral administration of the liquid composition of the invention to a subject does not result in any significant observed adverse events while achieving sufficient bioavailability of both cannabinoids to provide a biological response in the subject.

THC is the main psychotropic constituent of Cannabis, its main pharmacological effects including analgesia, muscle relaxation, antiemesis, appetite stimulation and psychoactivity. THC is a partial agonist of both the CB1 and CB2 receptors. THCs activity at the CB1 receptor are primarily implicated in its psychotropic effects. CB1 receptors are primarily expressed in the central nervous system, including within the brain, while CB2 receptors are typically expressed throughout the body, including in the peripheral nervous system. Further, due to its well-documented psychotropic efficacy, it is known that THC can cross the blood brain barrier when administered in a variety of forms, including inhalation and oral dosing.

Most of the recent data regarding the activity of THC focusses on the human endocannabinoid system. However, prior to the present invention it was commonly assumed that ingestion of THC by a companion animal, such as a dog or a cat, results in THC toxicity which includes symptoms of:
panting (in dogs), anxiety, and extreme agitation
dilated pupils, glossy-eyes, and a “dazed and confused” appearance
extreme lethargy
staggering, stumbling, and being unable to walk without falling or losing their balance
drooling and vomiting
diarrhea (especially if a pet has consumed high-fat edibles, cannabutter, or oils)
inability to control their bladders
abnormal heart rate and blood pressure.

CBD is the main non-psychotropic cannabinoid present in extracts of the Cannabis sativa plant, in some cases constituting up to 40 percent of its extract depending on plant genetics and the extraction technique employed. Both animal and human studies suggest that the pharmacokinetics and pharmacodynamics of CBD are very complex. CBD appears to operate at both CB1 and CB2 endocannabinoid receptors within the endocannabinoid system (ECS) indirectly stimulating endogenous cannabinoid signalling (anandamide) by suppressing fatty acid amide hydrolase (FAAH), the enzyme that breaks down anandamide. Importantly, this enables more anandamide to remain at the receptors, which elicits anxiolytic and antidepressant like effects. This indirect agonist property at the cannabinoid receptors may also explain its promising safety profile. Furthermore, CBD has been shown to also act on the vanilloid, adenosine and serotonin receptors explaining its broad spectrum of potential therapeutic properties in animal models and humans, including anxiolytic, antidepressant, neuroprotective, anti-inflammatory and immunomodulatory actions.

It is believed that THC and CBD used in combination act synergistically in the treatment of some indications, such as to maximize biological response. CBD has been demonstrated to antagonise some undesirable effects of THC including intoxication, sedation and tachycardia, while contributing analgesic, anti-emetic, and anti-carcinogenic properties. The liquid compositions of the invention therefore comprise the combination of THC and CBD to exploit the beneficial cooperation of these two active cannabinoids.

The composition of the invention is in a liquid form. Liquid form compositions may be preferred as the amount administered may be more readily adjusted depending on requirements for a subject compared to other dosage forms, such as tablets. In addition, liquid form compositions may also be preferred for oral dosing to subjects that may not be able to swallow solid compositions, such as non-human subjects. Accordingly, the liquid composition may be a pharmaceutical composition, a veterinary composition and/or a nutraceutical composition. The liquid composition is typically a solution and/or a suspension of the cannabinoids in an oral delivery system.

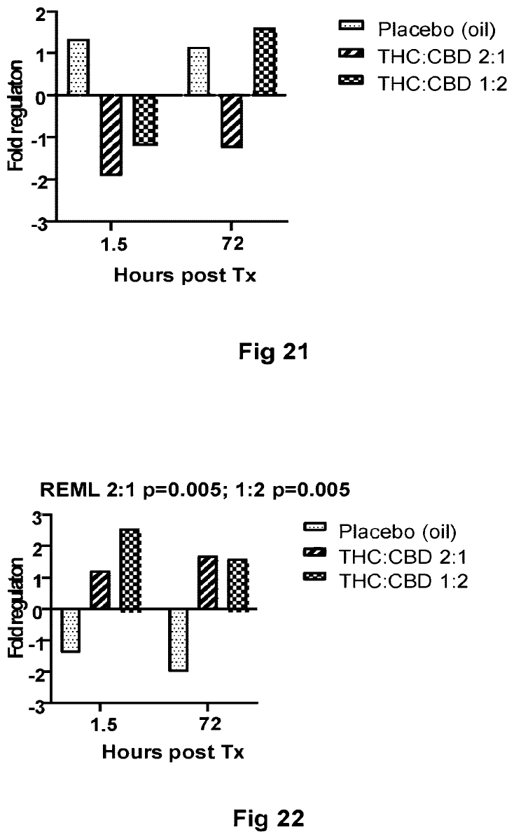

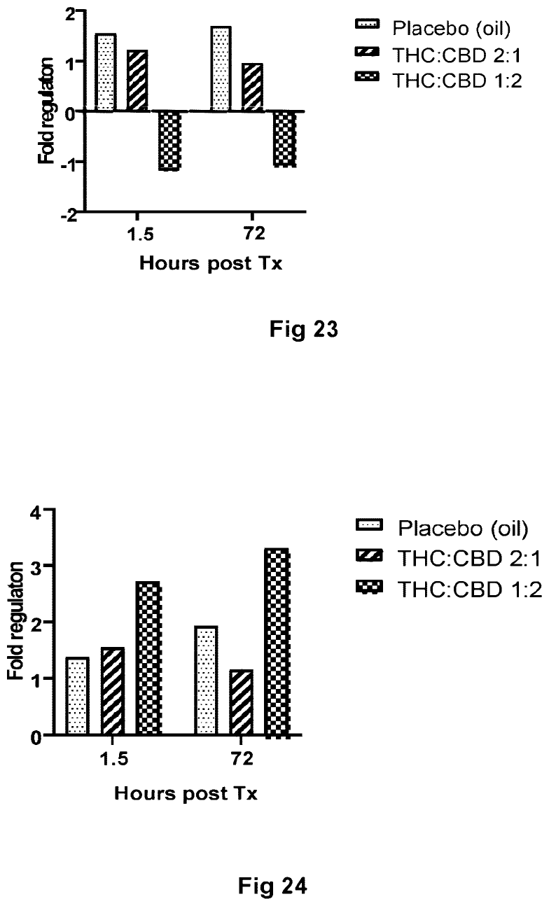

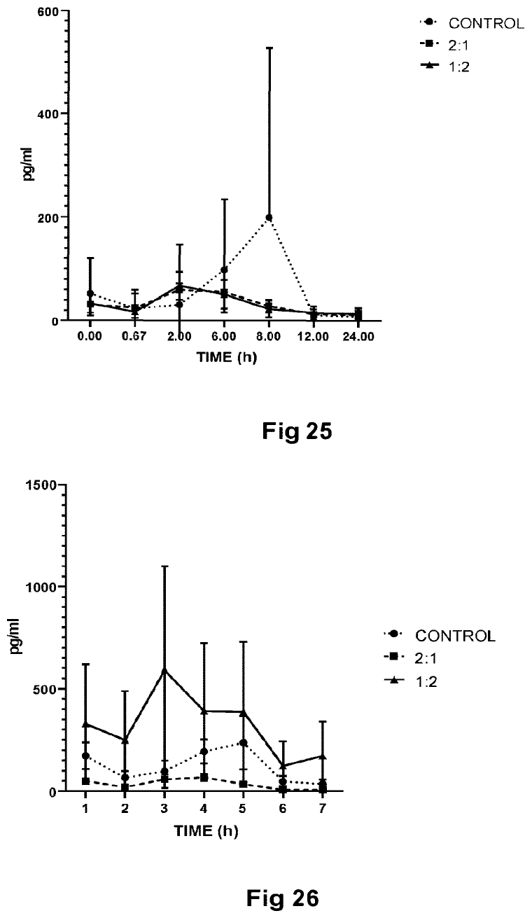

It was also surprisingly found that administering the liquid composition of the invention affected the gene expression profile (Example 4) and inflammatory biomarker concentrations (Example 5) for the subjects. Further, the expression of genes surprisingly differed depending on the ratio of THC:CBD contained in the composition (see FIGS. 20-26).

Accordingly, in some embodiments, the liquid composition comprises a higher amount as a proportion by weight of THC relative to CBD. Such liquid compositions may be referred to herein as “high-THC compositions”. The ratio by weight of THC:CBD in these embodiments may be greater than 1:1. For example, in some embodiments the ratio by weight of THC:CBD may be about 1.01:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.5:1, 4:1, 4.5:1, 5:1, 6:1, 7:1, 8:1, 9:1, 10:1, 20:1, 30:1, 40:1, 50:1, 60:1, 70:1, 80:1, 90:1, 95:1, 99:1 or higher. The ratio by weight of THC:CBD may be from any of these ratios to any other of these ratios without limitation. For example, the ratio by weight of THC:CBD may be from about 1.01:1 to about 99:1, about 1.01:1 to about 10:1 or about 1.1:1 to about 3:1.

Surprisingly, administration of a high-THC composition showed downregulation of CDR2 at 72 h after treatment and upregulation of CXCL8 at 1.5 h and 72 h after administration, which differed significantly from results obtained after administration of a vehicle (control) or compositions comprising a lower ratio of THC relative to CBD.

In other embodiments, the liquid composition comprises a lower amount as a proportion by weight of THC relative to CBD. Put another way, the liquid composition comprises a higher amount as a proportion by weight of CBD relative to THC in these embodiments. Such liquid compositions may be referred to herein as “high-CBD compositions”. In these embodiments, the ratio by weight of THC:CBD may be 1:1.2 or higher. For example, in some embodiments, the ratio by weight of THC:CBD may be about 1:1.2, 1:1.21, 1:1.25, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.5, 1:4, 1:4.5, 1:5, 1:6, 1:7, 1:8, 1:9, 1:10, 1:20, 1:30, 1:40, 1:50, 1:60, 1:70, 1:80, 1:90, 1:95, 1:99 or lower. The ratio by weight of THC:CBD may be from any of these ratios to any other of these ratios without limitation. For example, the ratio by weight of THC:CBD may be from 1:1.2 to about 1:99, about 1:1.3 to about 1:10 or about 1:1.4 to about 1:5.

Surprisingly, administration of liquid compositions comprising a higher ratio of CBD relative to THC showed upregulation of CCL5 at 1.5 h and 72 h after treatment, downregulation of CDR2 at 1.5 h after treatment, upregulation of CNR2 at 1.5 h after treatment at a level greater than following administration of a 2:1 THC:CBD composition, downregulation of CXCL8 at 1.5 h and 72 h after administration and greater than 3-fold upregulation of ADRB2. In addition, it was surprising that the concentrations for inflammatory biomarkers GM-CSF and IL-15 were elevated for the high-CBD compositions as compared to administration of the control and high-THC compositions.

It is believed that THC and CBD are artefacts of isolation and extraction from the Cannabis plant, where their natural biosynthesised precursors are Δ⁹-tetrahydrocannabinolic acid (THCA) and cannabidiolic acid (CBDA), respectively. Analytically it is also difficult to detect the precise concentration of THCA and CBDA due to their propensity to decompose into decarboxylated THC and decarboxylated CBD. In some embodiments, the liquid compositions comprising a non-natural amount of decarboxylated THC and decarboxylated CBD. In some embodiments, the liquid compositions comprise less than about 0.5 mg/ml THCA and/or CBDA.

In general, when cannabinoids are accessed from Cannabis extraction, the concentration of THC and THCA, and similarly the concentration of CBD and CBDA, are reported together due to the difficulty in preventing decarboxylation under analytical conditions, e.g. conditions associated with gas chromatography (GC), high-performance liquid chromatography (HPLC) or ultra-high-performance liquid chromatography (UPLC) techniques. Unless the context requires otherwise, any amount of THC referred to herein may be read as referring to the combined amount of THC and THCA. Unless the context requires otherwise, any amount of CBD referred to herein may be read as referring to the combined amount of CBD and CBDA. Further, unless the context requires otherwise, the relative amounts of THC:CBD described above may in some embodiments refer to the combined amount of THC and THCA relative to the combined amount of CBD and CBDA. In some embodiments, the liquid compositions may comprise the combination of THC, THCA, CBD and CBDA. The structures of THC, THCA, CBD and CBDA are shown in Table 1 below.

TABLE 1

| # | Cannabinoid | Structure |
|---|---|---|
| 1 | Δ⁹-Tetrahydrocannabinol (THC) | 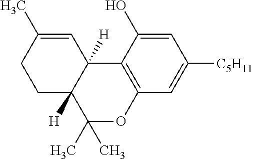 |
| 2 | Δ⁹-Tetrahydrocannabinolic acid (THCA) | 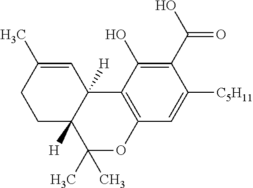 |
| 3 | Cannabidiol (CBD) | 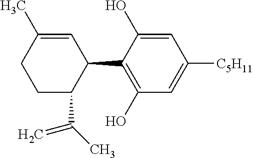 |
| 4 | Cannabidiolic acid (CBDA) | 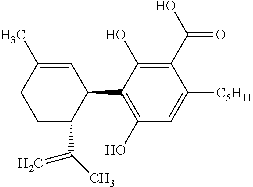 |

The composition may comprise any effective amount of THC. In some embodiments, the minimum amount of THC may be at least about 0.01%, 0.05%, 0.1%, 0.2%, 0.3%, 0.4% or 0.5%. In some embodiments, the maximum amount of THC may be not more than about 10%, 9%, 8%, 7%, 6%, 5%, 4%, 3%, 2%, 1%, 0.9%, 0.8%, 0.7% or 0.6%. The composition may comprise THC in an amount from any of these minimum amounts to any of these maximum amounts, for example, from about 0.01% to about 10% or about 0.1% to about 1%.

The composition may comprise any effective amount of CBD. In some embodiments, the minimum amount of CBD may be at least about 0.01%, 0.05%, 0.1%, 0.2%, 0.3%, 0.4% or 0.5%. In some embodiments, the maximum amount of CBD may be not more than about 10%, 9%, 8%, 7%, 6%, 5%, 4%, 3%, 2%, 1%, 0.9%, 0.8%, 0.7% or 0.6%. The composition may comprise CBD in an amount from any of these minimum amounts to any of these maximum amounts, for example, from about 0.01% to about 10% or about 0.1% to about 1%.

The THC and CBD present in the composition may be provided from a natural or a synthetic source, or a combination sources, including semi-synthetic sources. Cannabinoids provided from natural sources may be isolated, purified and then combined. Alternatively, an extract of a Cannabis plant (e.g. an extract that has been enriched in one or more of THC, THCA, CBD and CBDA) may be used to provide the cannabinoids. Synthetic or semi-synthetic or isolated natural THC, THCA, CBD and/or CBDA may be added to a Cannabis extract to enrich the extract in any of these cannabinoids. These mixtures may be prepared by any means known in the art.

In some embodiments, in addition to the combination of THC and CBD, the liquid compositions comprise one or more additional cannabinoids. The one or more additional cannabinoids may be selected from cannabigerol (CBG), cannabigerolic acid (CBGA), cannabinol (CBN) and cannabichromene (CBC). In other embodiments, the amount of any of these additional cannabinoids may be not more than about 1%, 0.7% or 0.5%, or the composition may be free of any or all of these additional cannabinoids.

In some embodiments, the combination of THC and CBD is provided in the form of a Cannabis extract.

To date, over 100 cannabinoids have been identified in Cannabis extracts. A comprehensive list of these cannabinoids may be found in Mahmoud A. El Sohly and Waseem Gul, “Constituents of Cannabis sativa” In Handbook of Cannabis Roger Pertwee (Ed.) Oxford University Press (2014) (ISBN: 9780199662685). Cannabinoids that have been identified in Cannabis plants include: Cannabigerol (E)-CBG-C5, Cannabigerol monomethyl ether (E)-CBGM-C5A, Cannabigerolic acid A (Z)-CBGA-C5 A, Cannabigerovarin (E)-CBGV-C3,Cannabigerolic acid A (E)-CBGA-C5 A, Cannabigerolic acid A monomethyl ether (E) CBGAM-C5 A and Cannabigerovarinic acid A (E)-CBGVA-C3 A; (+)-Cannabichromene CBC-C5, (+)-Cannabichromenic acid A CBCA-C5 A, (+)-Cannabivarichromene, (+)-Cannabichromevarin CBCV-C3, (+)-Cannabichromevarinic acid A CBCVA-C3 A; (−)-Cannabidiol CBD-C5, Cannabidiol monomethyl ether CBDMC, Cannabidiol-C4 CBD-C4, (−)-Cannabidivarin CBDV-C3, Cannabidiorcol CBD-CI, Cannabidiolic acid CBDA-C5, Cannabidivarinic acid CBDVA-C3; Cannabinodiol CBND-C5, Cannabinodivarin CBND-C3; Δ9-Tetrahydrocannabinol Δ9-THC-C5, Δ9-Tetrahydrocannabinol-C4 Δ9-THC-C4, Δ9-Tetrahydrocannabivarin Δ9-THCV-C3, Δ9-Tetrahydrocannabiorcol Δ9-THCO-CI, Δ9-Tetrahydrocannabinolic acid A Δ9-THCA-C5 A, Δ9-Tetrahydrocannabinolic acid B Δ⁹-THCA-C5 B, Δ⁹-Tetrahydrocannabinolic acid-C4 A and/or B Δ9-THCA-C4 A and/or B, Δ9-Tetrahydro-cannabivarinic acid A Δ⁹-THCVA-C3 A, Δ9-Tetrahydrocannabiorcolic acid A and/or B Δ9-THCOA-CI A and/or B, (−)-Δ8-trans-(6aR,10aR)-Δ8-Tetrahydrocannabinol Δ8-THC-C5, (−)-Δ8-trans-(6aR,10aR)-Tetrahydrocannabinolic acid A Δ8-THCA-C5 A, (−)-(6aS,10aR)-Δ9-Tetrahydrocannabinol (−)-cis-Δ9-THC-C5; Cannabinol CBN-C5, Cannabinol-C4 CBN-C4, Cannabivarin CBN-C3, Cannabinol C2 CBN-C2, Cannabiorcol CBN-CI, Cannabinolic acid A CBNA-C5 A, Cannabinol methyl ether CBNM-C5, (−)-(9R,10R)-trans-Cannabitriol (−)-trans-CBT-C5, (+)-(9S,10S)-Cannabitriol (+)-trans-CBT-C5, (±)-(9R,10S/9S,10R)-Cannabitriol (+)-cis-CBT-C5, (−)-(9R,10R)-trans-10-O-Ethyl-cannabitriol (−)-trans-CBT-OEt-C5, (±)-(9R,10R/9S,10S)-Cannabitriol-C3 (±)-trans-CBT-C3,8,9-Dihydroxy-46a (10a)-tetrahydrocannabinol 8,9-Di-OH-CBT-C5, Cannabidiolic acid A cannabitriol ester CBDA-C5 9-OH-CBT-C5 ester, (−)-(6aR,9S,10S,10aR)-9,10-Dihydroxyhexahydrocannabinol, Cannabiripsol, Cannabiripsol-C5, (−)-6a,7,10a-Trihydroxy-Δ9-tetrahydrocannabinol (−)-Cannabitetrol, 10-Oxo-Δ6a(10a)tetrahydrocannabinol (OTHC); (5aS,6S,9R,9aR)-Cannabielsoin CBE-C5, (5aS,6S,9R,9aR)-C3-Cannabielsoin CBE-C3, (5aS,6S,9R,9aR)-Cannabielsoic acid A CBEA-C5 A, (5aS,6S,9R,9aR)-Cannabielsoic acid B CBEA-C5 B; (5aS,6S,9R,9aR)-C3-Cannabielsoic acid B CBEA-C3 B, Cannabiglendol-C3 OH-iso-HHCV-C3, Dehydrocannabifuran DCBF-C5, Cannabifuran CBF-C5, (−)-Δ7-trans-(1R,3R,6R)-Isotetrahydrocannabinol, (±)-Δ7-1,2-cis-(1R,3R,6S/1S,3S,6R)-Isotetrahydrocannabivarin, (−)-Δ7-trans-(1R,3R,6R)-Isotetrahydrocannabivarin; (±)-(IaS,3aR,8bR,8cR)-Cannabicyclol CBL-C5, (±)-(1aS,3aR,8bR,8cR)-Cannabicyclolic acid A CBLA-C5 A, (±)-(IaS,3aR,8bR,8cR)-Cannabicyclovarin CBLV-C3; Cannabicitran CBT-C5; Cannabichromanone CBCN-C5, CannabichromanoneC3 CBCN-C3, and Cannabicoumaronone CBCON-C5.

The sum of the weights of any cannabinoids present in a Cannabis extract may be referred to herein as a cannabinoid fraction. The cannabinoid fraction typically accounts for the majority of the compounds present in the Cannabis extract. In some embodiments, the Cannabis extract comprises a cannabinoid fraction in a maximum amount of up to about 97%, 96%, 95%, 94%, 90%, 80%, 70% or 60%. The minimum amount of cannabinoid fraction may be at least about 15%, 20%, 30%, 40% or 50%. The Cannabis extract may comprise the cannabinoid fraction from any of these minimum amounts to any of these maximum amounts, for example, from about 15% to about 97% or about 50% to about 94%.

In addition to a cannabinoid fraction, Cannabis extracts may also comprise a diverse array of secondary metabolites, including terpenes and terpenoids, sterols such as phytosterols, triglycerides, alkanes, squalenes, tocopherols, carotenoids, flavonoids, polyphenols, cannflavins and alkaloids. The mix of these secondary metabolites varies depending on several factors, including Cannabis variety, part of the Cannabis plant extracted, method of extraction, processing of the extract, and season. In some embodiments, the Cannabis extract may comprise the non-cannabinoid fraction in a maximum amount of up to about 20%, 15%, 10%, 6% or 5%. The Cannabis extract may comprise the non-cannabinoid fraction in a minimum amount of at least about 0.001%, 0.005%, 0.01%, 0.05%, 0.1%, 0.5%, 1% or 3%. The Cannabis extract may comprise the non-cannabinoid fraction in an amount from any of these minimum amounts to any of these maximum amounts, for example, from about 0.001% to about 20% or about 0.1% to about 6%.

There are several varieties of Cannabis plant, which have been described under two distinct naming conventions. One of these conventions identifies three distinct species of Cannabis plant, namely Cannabis sativa Linnaeus, Cannabis indica LAM., and Cannabis ruderalis. Another convention identifies all Cannabis plants as belonging to the Cannabis sativa L. species, with the various varieties divided amongst several subspecies, including: Cannabis sativa ssp. sativa and ssp. indica. As used herein, the term “Cannabis” refers to any and all of these plant varieties.

The Cannabis extract may be prepared by any means known in the art. The extracts may be formed from any part of the Cannabis plant. Extracts may be formed by contacting an extractant with a leaf, seed, trichome, flower, kief, shake, bud, stem or a combination thereof. Any suitable extractant known in the art may be used, including, for example, alcohols (e.g. methanol, ethanol, propanol, butanol, propylene glycol, etc.), water, hydrocarbons (e.g. butane, hexane, etc.), oils (e.g. olive oil, vegetable oil, etc.), a polar organic solvent (e.g. ethyl acetate, polyethylene glycol, etc.) or a supercritical fluid (e.g. liquid CO₂). The extractant may be completely or partially removed prior to incorporation of the Cannabis extract into the composition, or it may be included in the composition, and in some embodiments may form part of the oral delivery system. The extractant may be removed by heating the extract optionally under reduced pressure (e.g. under vacuum). It will be appreciated that some of the more volatile plant metabolites (such as terpenes) may also be removed with the extractant and some cannabinoids may decompose under heating, such as decarboxylation of a cannabinoid acid (e.g. THCA or CBDA). In some embodiments, removing the extractant may enrich the cannabinoid fraction of the extract. In some embodiments, the extract is filtered to remove particulate material, for example, by passing the extract through filter paper or a fine sieve (e.g. a sieve with pore sizes of 5 μm).

In some embodiments, the Cannabis extract is formed by applying heat and pressure to the plant material. Typically, in these embodiments, no extractant is required.

### Oral Delivery System

The liquid composition comprises an oral delivery system. The oral delivery system comprises one or more pharmaceutically, veterinary and/or nutraceutically acceptable carriers to solubilise or suspend the THC and CBD in a manner that enables it to be administered orally to a subject.

The composition may comprise the oral delivery system in a major amount. In some embodiments, the composition comprises a minimum concentration of oral delivery system of at least about 40%, 45%, 50%, 55%, 60%, 65%, 70%, 75%, 80%, 85%, 90%, 91%, 92%, 93%, 94%, 95% or 96%. The composition may comprise a maximum concentration of oral delivery system of up to about 99.99%, 99.9%, 99.5%, 99%, 98.5, 98%, 97.5%, 97% or 96.5%. The concentration of fatty acid component in the composition may be from any of these minimum values to any of these maximum values, for example from about 40% to about 99.99% or about 75% to about 98%.

The oral delivery system preferably comprises all liquid ingredient(s). In some embodiments, the oral delivery system comprises a liquid ingredient selected from ethanol, glycerine, propylene glycol, a fatty acid (optionally in the form of an oil), an emulsifier, and water or a combination thereof.

Suitable emulsifiers include cationic, anionic, zwitterionic and non-ionic surfactants. In some embodiments, the emulsifier may be selected from the following group: a sulfate ester, a sulfonate ester, a phosphate ester, an alkyl carboxylate, lecithin, a quaternary ammonium salt, a polyethylene glycol (PEG), an alkylated fatty acid and/or fatty acid ester, a fatty acid ester of a polyol (such as sorbitan monolaurate, sorbitan monostearate and sorbitan tristearate), an alkylpolyglucoside, an ethoxylated amine, an ethoxylated fatty acid amide, a poloxamer, a polysorbate (e.g. polysorbate 20, 40, 60 or 80 or tween 20, 40, 60 or 80), a pegylated oil (e.g. polyoxyl 35 castor oil), and a combination thereof.

As THC and CBD are both lipid soluble, in some embodiments, the oral delivery system comprises a fatty acid. The fatty acid may be in the form of a free acid or an ester, such as a glyceride. It will therefore be understood that a reference to a “fatty acid” includes the free fatty acid and an ester thereof. Fatty acid esters include glyceryl esters, such as monoglycerides, diglycerides and triglycerides. The fatty acid residues included in a diglyceride and/or triglyceride may be the same or different and may be selected from any of the fatty acids described herein.

The fatty acid may be selected from saturated and unsaturated fatty acids or a combination thereof.

Unsaturated fatty acids may comprise from 1 to n/2 double carbon-carbon bonds, wherein n is the number of carbon atoms in the fatty acid side chain. Typically, unsaturated fatty acids comprise from 1 to 10 double carbon-carbon bonds. The double carbon-carbon bonds may be cis or trans. Typically, the double carbon-carbon bonds are cis.

The fatty acid may be:
a short chain fatty acid (SCFA) comprising from 2 to 6 carbon atoms (inclusive of the carboxyl carbon);
a medium-chain fatty acid (MCFA) comprising from 7 to 13 carbon atoms (inclusive of the carboxyl carbon);
a long-chain fatty acid (LCFA) comprising from 14 to 22 carbon atoms (inclusive of the carboxyl carbon); and/or
a very long chain fatty acid (VLCFA) comprising 23 or more carbon atoms (inclusive of the carboxyl carbon), for example from 23 to 100 carbon atoms.

In some embodiments, the fatty acid component comprises a MCFA, a LCFA or a combination thereof.

In some embodiments, the fatty acid component comprises a fatty acid selected from one or more of the group consisting of butyric acid (4:0); caproic acid (6:0); caprylic acid (8:0); capric acid (10:0); undecanoic acid (11:0); lauric acid (12:0); tridecanoic acid (13:0); myristic acid (14:0); myristoleic acid (14:1); pentadecanoic acid (15:0); cis-10-pentadecanoic acid; cis-10-pentadecenoic acid; palmitic acid (16:0); palmitoleic acid (16:1n-9); hexadecenoic acid (16:1); hexadecadienoic acid (16:2); margic/heptadecanoic acid (17:0); cis-10-heptadecanoic acid; cis-10-heptadecenoic acid; margaroleic acid (17:1); stearic acid (18:0); vaccenic acid (18:1); oleic acid (18:1); elaidic acid (18:1); linoleic acid (LA; 18:2); linolelaidic acid (18:2n-6); linolenic acid (18:3) including a-linolenic acid (ALA) and γ-linolenic acid (GLA); octadecatrienoic acid (18:3); stearidonic acid (SDA; 18:4n-3); arachidic acid (20:0); eicosenoic acid (20:1) including gadoleic acid (20:1n-11), gondoic acid (20:1n-9) and paullinic acid (20:1n-7); eicosadienoic acid (20:1n-6); cis-11,14,17-eicosatrienoic acid; cis-8,11,14-eicosatrienoic acid; eicosatetraenoic acid; arachidic acid (AA; 20:0); eicosapentaenoic acid (20:5n-3); heneicosanoic acid (21:0); behenic acid (22:0); cetoleic/erucic acid (22:1n-9); dicosadienoic acid (22:2n-6); docosapentanoic/docosapentaenoic acid (DPA; 22:5); docosahexaenoic acid (DHA; 22:6n-3); tricosanoic acid (23:0); lignoceric acid (24:0); and nervonic acid (24:1n-9).

The fatty acid component may comprise fatty acids derived from nature or produced synthetically (i.e. non-natural). In some embodiments, the liquid composition comprises at least one fatty acid from a non-natural source.

In some embodiments, the fatty acid is provided in the form of an edible oil. In some embodiments, the edible oil is selected from flax seed oil, hemp seed oil, fish oil, coconut oil, cocoa butter, palm kernel oil, palm oil, cottonseed oil, wheat germ oil, soybean oil, olive oil, corn oil, sunflower oil, safflower oil, canola oil, sesame oil, peanut oil, rosemary oil, anise oil, or a combination thereof. In some embodiments, the fatty acid component comprises a combination of 2, 3, 4 or more oils, such as any of the oils described herein.

Triglycerides comprising at least 1 MCFA may be referred to herein as a medium chain triglyceride (MCT). In some embodiments, the oral delivery system comprises a MCT. In these embodiments, the MCT may be provided in the form of an oil comprising an MCFA, herein referred to as an MCT oil. MCT oils are advantageously associated with a range of health benefits when administered to a subject, including when administered in the composition of the invention.

In some embodiments, wherein the oral delivery system comprises a fatty acid, the composition may further comprise an antioxidant to delay or prevent oxidation of the fatty acid. Any compatible antioxidant may be included. The antioxidant may be selected from ascorbic acid, butylated hydroxyanisole, butylated hydroxytoluene (BHT), propyl gallate, a tocopherol and a pentacyclic triterpenic acid (such as those described in Australian Provisional Patent Application No. 2019901093) or a combination thereof.

The composition may comprise the antioxidant in a stabilising amount. A stabilising amount is an amount effective to delay oxidation of the fatty acid component.

In some embodiments, the minimum concentration of antioxidant may be at least about 0.0001%, 0.0005%, 0.001%, 0.005%, 0.01%, 0.05% or 0.1%. In some embodiments, the maximum concentration of antioxidant may be not more than about 20%, 15%, 10%, 9%, 8%, 7%, 6% or 5%. The composition may comprise the antioxidant in a concentration from any of these minimum concentrations to any of these maximum concentrations, for example, from about 0.0001% to about 20% or from about 0.05% to about 6%.

In some embodiments, the composition may comprise a further active pharmaceutical, veterinary and/or nutraceutical ingredient other than THC and CBD. Any compatible active pharmaceutical, veterinary and/or nutraceutical ingredient may be included. In some embodiments, the further active ingredient may be selected from any one or more of the following: anti-inflammatoirenalgesics, opiates, anti-convulsants, antibiotics, anti-helmintic, steroids and hormones, anaesthetic agents, sedatives, anti-ulcer, anti-emetics, tranquilisers, antiparasitic agents, bronchodilators, decongestants, reflux medications, anti-pyretics, diuretics, antiparasitic agents, antiprutitic agents, and sympathomimetic agents.

When present, the further active pharmaceutical, veterinary and/or nutraceutical ingredient is included in a therapeutically useful amount which is sufficient to provide a suitable dosage to a subject following administration. Accordingly, the composition may comprise an effective amount of a further active pharmaceutical, veterinary and/or nutraceutical ingredient.

In some embodiments, each further active pharmaceutical, veterinary and/or nutraceutical ingredient is present in a minimum amount of at least about 0.0001%, 0.0005%, 0.001%, 0.005%, 0.01% or 0.05%. In some embodiments, each further active pharmaceutical, veterinary and/or nutraceutical ingredient is present in a maximum amount of not more than about 10%, 5%, 1% or 0.5%. The composition may comprise each further active pharmaceutical, veterinary and/or nutraceutical ingredient from any of these minimum amounts to any of these maximum amounts, for example, from about 0.0001% to about 10% or from about 0.01% to about 1%.

References to the various compounds described herein, such as THC and CBD, include the relevant compound and pharmaceutically, veterinary and/or nutraceutically acceptable salts, tautomers and solvates thereof.

The various compounds may be provided as salts which are pharmaceutically, veterinary and/or nutraceutically acceptable. Examples of pharmaceutically and veterinary acceptable salts include salts of pharmaceutically, veterinary and/or nutraceutically acceptable cations such as sodium, potassium, lithium, calcium, magnesium, ammonium and alkylammonium; acid addition salts of pharmaceutically, veterinary and/or nutraceutically acceptable inorganic acids such as hydrochloric, orthophosphoric, sulfuric, phosphoric, nitric, carbonic, boric, sulfamic and hydrobromic acids; or salts of pharmaceutically, veterinary and/or nutraceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, trihalomethanesulfonic, toluenesulfonic, benzenesulfonic, isethionic, salicylic, sulphanilic, aspartic, glutamic, edetic, stearic, palmitic, oleic, lauric, pantothenic, tannic, ascorbic, valeric and orotic acids. Salts of amine groups (if present) may also comprise quaternary ammonium salts in which the amino nitrogen atom carries a suitable organic group such as an alkyl, alkenyl, alkynyl or aralkyl moiety.

The salts may be formed by conventional means, such as by reacting the free base form of the compound with one or more equivalents of the appropriate acid.

It should be understood that a reference to a pharmaceutically, veterinary and/or nutraceutically acceptable salt includes the solvent addition forms or crystal forms thereof, particularly solvates or polymorphs.

A “tautomer” is a structural isomer of a compound that is in equilibrium with another of the compound's structural isomers. This equilibrium is typically driven by thermodynamics making isolation of only one tautomer of a compound that exhibits tautomerism impossible by conventional techniques. To the extent that any of the present compounds exhibit tautomerism, it is intended that the invention includes all tautomers of the various compounds and derivatives thereof.

The compound(s) may exist in unsolvated as well as solvated forms with acceptable solvents such as water, ethanol, and the like. Solvates contain either stoichiometric or non-stoichiometric amounts of a solvent. Hydrates are formed when the solvent is water. Alcoholates are formed when the solvent is an alcohol. In general, the solvated forms are considered equivalent to the unsolvated forms for the purposes of the compositions and methods provided herein.

The liquid compositions may additionally comprise a pharmaceutically, veterinary and/or nutraceutically acceptable additive. The additive may be selected from the group consisting of one or more colorants, bulking agents, excipients, binders, preservatives, flavouring agents, buffers, artificial and natural sweeteners, dispersants, thickeners and solubilising agents, and combinations thereof. The additive may be any additive included in the United States Pharmacopeia/National Formulary (USP/NF), the British Pharmacopoeia (BP), the European Pharmacopoeia (EP), the Japanese Pharmacopoeia (JP) or the Chinese Pharmacopoeia (ChP). In some embodiments, the composition comprises an additive which may be non-natural (e.g. synthetically produced).

The liquid compositions may be formulated according to techniques such as those well known in the art of pharmaceutical, veterinary and/or nutraceutical formulation (see, for example, Remington: The Science and Practice of Pharmacy, 21st Ed., 2005, Lippincott Williams & Wilkins and/or Veterinary pharmacology and therapeutics, Riviere, J. (Ed.); Papich, Mark G., (Ed.); Wiley-Blackwell; 2017).

The pharmaceutical, veterinary and/or nutraceutical compositions may be prepared in unit dosage form. In such form, the compositions are subdivided into unit doses containing appropriate quantities of the ingredient(s). The unit dosage form can be a packaged preparation, the package containing discrete quantities of preparation. The preparation may be a solution, suspension, emulsion, elixir, tincture or capsule filled with a solution, suspension, emulsion, elixir or tincture.

For preparing pharmaceutical, veterinary and/or nutraceutical compositions described herein, pharmaceutically, veterinary and/or nutraceutically acceptable additives can be either solid or liquid. In the case of solid additives, these should be soluble and/or dispersible within the liquid components of the liquid composition such that the composition is provided in liquid form.

The liquid compositions of the invention may be sterile. Sterile liquid form compositions include sterile solutions, suspensions, emulsions, syrups and elixirs.

Typically, the administration of the compositions is oral administration. The compositions may be formulated for oral administration in any suitable form. For example, compositions for oral administration may be formulated in one or more of the following liquid forms: a solution, a suspension, an emulsion, an elixir, a syrup, or a capsule filled with a solution, a suspension, an emulsion, an elixir, a syrup, a tincture or a combination thereof.

In some embodiments, the dosage unit form may be a capsule. To form a capsule, typically the ingredient(s) are combined with one or more of pharmaceutically, veterinary and/or nutraceutically acceptable excipients (e.g. carriers) to provide a liquid composition, which is then encased within a capsule shell. Any suitable capsule shell known in the art may be used, including hard and soft capsule shells. Suitable hard capsule shells may comprise gelatine, HPMC, starch, pullulan and/or polyvinyl acetate (PVA). Suitable soft capsules may comprise gelatin thickened with a thickening agent, such as a polyol (e.g. glycerine or sorbitol). As noted above, the capsule shell may be filled with any of the following dosage forms described herein: a solution, a suspension, an emulsion, an elixir, a syrup, or a combination thereof.

Various other materials may be present as coatings or to otherwise modify the physical form of the dosage unit. For instance, capsules may be coated with shellac, sugar or both. Of course, any material used in preparing any dosage unit form should be pharmaceutically, veterinary and/or nutraceutically acceptable and substantially non-toxic in the amounts employed. In addition, the liquid composition may be incorporated into sustained-release preparations and formulations.

Also included are preparations that are intended to be diluted, shortly before use. In such cases, a concentrated form of the liquid composition of the invention (e.g. a form lacking all or part of an oral delivery system) may be diluted with a liquid carrier (such as an oral delivery system) shortly before use. Alternatively, a liquid composition of the invention may be further diluted with a liquid carrier shortly before use.

In some embodiments, a liquid composition of the invention is incorporated into a food product. Accordingly, in another aspect, there is provided a food product comprising the liquid composition of the invention. It will be appreciated that the food product is not limited to liquids. Suitable food products include solid food products including baked goods and liquid food products such as a beverage. The composition may be incorporated into the food product during manufacture or may be added to an existing product. Therefore, the food products disclosed herein may further comprise at least one Generally Recognised As Safe (GRAS) ingredient. The GRAS ingredient may be any ingredient included in the GRAS database maintained by the US Food and Drug Administration (FDA). In some embodiments, the food product is an animal treat, such as a dog biscuit.

The food product may be a functional food product, wherein the food product also comprises a pharmaceutically, veterinary and/or nutraceutically active ingredient. Any of the active pharmaceutical, veterinary and/or nutraceutical ingredients described above may be included in the functional food. The active pharmaceutical, veterinary and/or nutraceutical ingredient may be included as a part of the composition, or may be incorporated separately into the functional food product.

Pharmaceutically, veterinary and/or nutraceutically acceptable carriers and/or diluents include any and all solvents, dispersion media, coatings, antibacterial and antifungal agents, isotonic and absorption delaying agents and the like

When desired, formulations adapted to give sustained release of the active ingredient(s) may be employed. In addition, the liquid compositions may be formulated to provide a self-emulsifying drug delivery system (SEED), a lipid-based drug-delivery system (LBDDS) and/or a self-microemulsifying drug delivery system (SMEDDS).

The practice of the present invention employs, unless otherwise indicated, conventional pharmaceutical and/or medical techniques within the skill of the art. Such techniques are well known to the skilled worker, and are explained fully in the literature.

Also provided is a method of preparing the liquid composition of the invention. The method comprises:
preparing a THC solution comprising THC and a liquid carrier;
preparing a CBD solution comprising CBD and a liquid carrier; and
combining an aliquot of the THC solution with an aliquot of the CBD solution to provide a liquid composition comprising a combination of THC and CBD.

Typically, the THC solution comprises a first concentration of THC and the CBD composition comprises a second concentration of CBD. The first and second concentrations may be predetermined or assessed through an analytical technique. Any suitable analytical technique may be employed, including GC, HPLC and UPLC.

Preparing a liquid composition according to this method allows for the control of the final ratio of THC:CBD. If either the THC or CBD is obtained by extraction of Cannabis plant material, the concentration of the THC or CBD may vary from extract to extract. Regardless of source of the THC and CBD, this method allows adjustment of the volume of the aliquots to ensure the final ratio of THC and CBD in the liquid composition.

The liquid carrier of the THC and/or CBD composition may comprise an oral delivery system. Any oral delivery system described herein may be used.

In some embodiments, the method further comprises dilution of the liquid composition. The liquid composition may be diluted with an oral delivery system. Any oral delivery system described herein may be used.

The liquid composition produced by this method may be any of the liquid compositions described herein.

### Methods of Treatment

The liquid composition of the invention may be used to treat any disease, disorder or condition associated with the endocannabinoid system. THC and CBD therapy have been indicated as useful in the treatment of one or more of the following disease, disorders and conditions: pain control—post operative, arthritis, cancer, neuropathic, injury, anxiety relief, epilepsy treatment, gastro-intestinal health conditions, improved skin health—atopic dermatitis, itching, appetite stimulation, palliative care, anti-inflammatory, alleviates glaucoma, improved joint and bone health, aid for sleep, nervous system support including OCD, depression, migraines, autism, an allergy (e.g. a contact allergy, a food allergy or any other allergy), reduced vomiting and nausea, helps suppress muscle spasms, management of phobias, management of behaviour disorders, management of cognitive dysfunction, reduction in cancer growth, killed or slowed bacteria/fungal growth, reduced blood sugar levels and imbalances of fatty acids.

In particular, the invention provides a method of treating pain, inflammation and/or anxiety. The method comprises administering to a subject in need thereof an effective amount of the liquid composition of the invention. Any of the liquid compositions of the invention described herein may be employed in these methods.

The pain treated in the present methods may be nociceptive pain, psychogenic pain and/or neuropathic pain. Nociceptive pain is associated with stimulation of sensory nerve endings (or nociceptors). Psychogenic pain is associated with psychological factors resulting in a pain disorder (often diagnosed when other physical causes for pain are ruled out). Neuropathic pain is associated with damage or malfunction of the peripheral nervous system (PNS) or central nervous system (CNS). Cannabinoid receptors (e.g. CB1 and CB2 receptors) have been reported as being expressed in the PNS and CNS. The pain to be treated may be any pain associated with the endocannabinoid system.

The inflammation treated in the present methods may be any form of inflammation associated with the activity of the endocannabinoid system. The inflammation may be localised or systemic.

In some embodiments, the inflammation may be a symptom or a cause a disease and/or disorder. The disease and/or disorder may be selected from osteoarthritis (OA), rheumatoid arthritis (RA), psoriatic arthritis (PsA), ankylosing spondylitis (AS), non-radiographic axial spondyloarthritis (nr-axSpA), idiopathic arthritis, anterior knee pain, chilblains, chronic recurrent multifocal osteomyelitis, fibromyalgia, familial Mediterranean fever (FMF), gout, growing pains, haemochromatosic arthritis, localised scleroderma, lupus, polymyalgia rheumatica, reactive arthritis, ross river fever, scleroderma, Sever's disease, Sjogren's syndrome and spondyloarthritis, or a combination thereof.

The anxiety treated in the present methods may be any form of anxiety associated with the endocannabinoid system or associated with a subject's experience of a disease and/or condition associated with the endocannabinoid system such as an inflammatory disease and/or disorder, and/or pain. The anxiety may be generalised anxiety or a specific anxiety (e.g. a phobia, anxiety to sound or travel, separation anxiety, etc.). Anxiety is experienced by humans and non-human animals alike. In dogs, for example, one measure of anxiety reduction is heart rate. It was surprisingly found that for embodiments of this invention comprising a lower amount of THC relative to CBD that the average heart rate of the subjects was reduced across the treatment period.

The method comprises administering an effective amount of the liquid composition of the invention. The effective amount may be determined by the skilled person based on numerous factors, including the severity and kind of symptoms of the subject, the subject's medical history, the subject's physical attributes (weight, sex, etc), the specific combination of active ingredients included in the pharmaceutical compositions to be administered.

In some embodiments, the liquid composition is administered to the subject to provide a minimum dose of THC of at least about 0.001 mg/kg, 0.005 mg/kg, 0.01 mg/kg, 0.05 mg/kg, 0.1 mg/kg, 0.12 mg/kg, 0.15 mg/kg or 0.2 mg/kg. The maximum dose of THC provided to the subject may be not more than about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.4 mg/kg, 0.3 mg/kg, 0.25 mg/kg or 0.24 mg/kg. The dose of THC administered to the subject may be from any of these minimum amounts to any of these maximum amounts, for example, from about 0.001 mg/kg to about 10 mg/kg, about 0.01 mg/kg to about 1 mg/kg, about 0.1 mg/kg to about 0.3 mg/kg or about 0.12 mg/kg to about 0.24 mg/kg.

In some embodiments, the liquid composition is administered to the subject to provide a minimum dose of CBD of at least about 0.001 mg/kg, 0.005 mg/kg, 0.01 mg/kg, 0.05 mg/kg, 0.1 mg/kg, 0.12 mg/kg, 0.15 mg/kg or 0.2 mg/kg. The maximum dose of CBD provided to the subject may be not more than about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.4 mg/kg, 0.3 mg/kg, 0.25 mg/kg or 0.24 mg/kg. The dose of CBD administered to the subject may be from any of these minimum amounts to any of these maximum amounts, for example, from about 0.001 mg/kg to about 10 mg/kg, about 0.01 mg/kg to about 1 mg/kg, about 0.1 mg/kg to about 0.3 mg/kg or about 0.12 mg/kg to about 0.24 mg/kg.

Any of the doses of THC and CBD given above may be combined without limitation, and may be dictated, for example, on the ratio of THC:CBD contained in the composition.

The liquid compositions of the invention may be administered with or without food. In some embodiments, the subject is fasted for at least about 3, 6, 8, 10 or 12 hours prior to administration. In other embodiments, the liquid composition may be administered within about 0.5, 1 or 2 hours of consuming food.

The method comprises administering at least two active ingredients, namely THC and CBD optionally together with a further active ingredient. While in some embodiments, the THC and CBD are formulated into the same a liquid composition, some embodiments of these methods may involve administering the active compounds simultaneously, separately or consecutively. By simultaneously it is meant that each of the active ingredients are administered at the same time in the same composition. By separately it is meant that each of the active ingredients are administered at the same time in different compositions and optionally by different routes of administration. By consecutively it is meant that each of the composition and the further active ingredient are administered separately optionally by different administration routes and may be at different times. Typically, when the active ingredients are administered consecutively, they are administered within 24 hours, or within 12, 8, 6, 5, 4, 3, 2, or 1 hour(s) of each other. THC may be administered before or after the CBD.

The THC and CBD may be administered 1, 2, 3, 4 or more times per day as required based on the disease, disorder and/or condition to be treated. Typically, the composition is administered once daily or twice daily (BID).

In some embodiments, the peak THC plasma concentration may be achieved within about 3, 2, 1.5 or 1 hours after administration. The peak THC plasma concentration may occur after at least about 0.2, 0.3, 0.4, 0.5, 0.6 or 0.67 hours after administration. The peak THC plasma concentration may be achieved from about 0.2 hours to about 3 hours after administration or about 0.67 hours to about 1.5 hours after administration.

In some embodiments, the peak CBD plasma concentration may be achieved within about 3, 2, 1.5 or 1 hours after administration. The peak CBD plasma concentration may occur after at least about 0.2, 0.3, 0.4, 0.5, 0.6 or 0.67 hours after administration. The peak CBD plasma concentration may be achieved from about 0.2 hours to about 3 hours after administration or about 0.67 hours to about 1.5 hours after administration.

While the absorption of THC and CBD from the composition may be considered independent even when dosed simultaneously, in some embodiments, the peak plasma concentration for both THC and CBD is achieved within about 0.5, 0.4, 0.3 or 0.2 hours.

In some embodiments, the peak THC plasma concentration (Cₘₐₓ) achieved was from about 19 ng/ml to about 71.3 ng/ml. Surprisingly, it was observed that THC was detectable in the subject's blood 72 h after administration. Accordingly, in some embodiments, the THC plasma concentration may be at least about 0.11 ng/ml, 0.22 ng/ml, 0.28 ng/ml, 0.34 ng/ml, 0.47 ng/ml, 0.58 ng/ml, 1.48 ng/ml, 1.91 ng/ml, 10.5 ng/ml, 71.3ng/mL. The plasma THC concentration achieved may be from any of these concentrations to any other of these concentrations, for example, from about 0.11 ng/ml to about 71.3 ng/ml, about 0.22 ng/ml to about 0.58 ng/ml, about 1.48 ng/ml to about 10.5 ng/ml, about 0.47 ng/mL to about 1.91 ng/ml, or about 0.11 ng/mL to about 0.46 ng/ml.

In some embodiments, the peak CBD plasma concentration (Cₘₐₓ) achieved was from about 5 ng/ml to about 69 ng/mL. Surprisingly, it was observed that CBD was detectable in the subject's blood up to 72 h after administration. Accordingly, in some embodiments, the CBD plasma concentration may be at least about 0.23 ng/ml, 0.26 ng/ml, 0.28 ng/ml, 0.46, ng/ml, 0.76 ng/ml, 0.88 ng/mL, 5.74 ng/ml, 69 ng/mL. The plasma CBD concentration achieved may be from any of these concentrations to any other of these concentrations, for example, from about 0.23 ng/ml to about 69 ng/ml, about 0.76 ng/ml to about 5.74 ng/ml, about 0.11 ng/ml to about 0.46 ng/mL or about 0.23 ng/ml to about 0.88 ng/ml.

Any of the THC and/or CBD plasma concentrations described herein, without limitation, may be maintained after administration for at least about 6, 12, 18, 24, 32 hours or longer.

In addition to plasma concentration, it is also useful to calculate the area under the curve (AUC) for an administered active ingredient. In some embodiments, the AUC for THC concentration after administering a dose of 0.12 mg/kg THC in combination with 0.24 mg/kg CBD to a canine subject may be from about 90 to about 110, for example about 95 to about 100. The AUC for THC concentration after administering a dose of 0.24 mg/kg THC in combination with 0.12 mg/kg CBD to a canine subject may be from about 145 to about 170, for example about 150 to about 160. In some embodiments, the AUC for CBD concentration after administering a dose of 0.12 mg/kg CBD in combination with 0.24 mg/kg THC to a canine subject may be from about 30 to about 50, for example about 35 to about 45. The AUC for CBD concentration after administering a dose of 0.24 mg/kg CBD in combination with 0.12 mg/kg THC to a canine subject may be from about 95 to about 110, for example about 100 to about 105. These AUC results show that both THC and CBD achieve measurable plasma concentrations after oral dosing of the liquid composition of the invention. Surprisingly, the AUC of THC following administration of 0.24 mg/kg THC/0.12 mg/kg CBD is less than 2 times that of the AUC of the AUC of THC following administration of 0.12 mg/kg THC/0.24 mg/kg CBD. Similarly, it was surprisingly found that the AUC of CBD achieved following administration of 0.12 mg/kg THC/0.24 mg/kg CBD is more than twice that of the AUC of CBD following administration of 0.24 mg/kg THC/0.12 mg/kg CBD. This relative improvement in bioavailability for both THC and CBD suggest a synergistic effect in terms of oral absorption for the high-CBD compositions of the invention.

The subject to be treated may be a human or a non-human subject. Non-human subjects may be any animal possessing an endocannabinoid system. It is believed that the endocannabinoid system is highly conserved across all vertebrates as well as in some invertebrates too. The term “animal” as used herein includes but is not limited to companion animals, food-production animals and zoo animals. Companion animals include dogs, cats, guinea pigs, hamsters and horses. Food-production animals include cattle, goats, sheep, fowl, poultry and swine. Zoo animals include monkeys, elephants, giraffes and other ungulates, bears, mice and other small mammals. Typically, the subject is a dog or cat, most typically a dog.

In another aspect, the invention provides a method of controlling the heart rate of a subject. The method comprises orally administering to the subject an effective amount of a liquid composition of the invention.

In some embodiments, controlling the heart rate means lowering the average heart rate of the subject for a period of time following the oral administration. In these embodiments, the liquid composition may be a high-CBD composition.

The liquid composition may be dosed in any of the effective amounts described herein. The pharmacokinetic profile for the THC and CBD following administration may therefore also be any plasma concentration profile described herein.

In some embodiments, controlling the heart rate means preventing elevation of the average heart for a period of time following administration.

The period of time following administration may be at least about 2, 3, 4, 5, 6, 7, 8, 9, 10, 11, 12, 14, 16, 18, 20, 24 hours or longer, for example up to about 72 hours.

Also disclosed herein is a kit of parts comprising in separate parts:
(a) a liquid composition comprising THC and an oral delivery system; and
(b) a liquid composition comprising CBD and an oral delivery system.

The oral delivery system of parts (a) and (b) may be any oral delivery system described herein. Typically, the oral delivery system of parts (a) and (b) is the same. In some embodiments, parts (a) and (b) may be combined to provide a liquid composition of the invention.

### Gene Expression

One aspect of the invention provides a method of modulating the expression of one or more genes selected from chemokine (C-C motif) ligand 5 (CCL5, as shown in SEQ ID NO:4), cerebellar degeneration-related protein 2 (CDR2, as shown in SEQ ID NO:2), cannabinoid receptor 2 (macrophage) (CNR2, as shown in SEQ ID NO:3), interleukin 8 (CXCL8, as shown in SEQ ID NO:1) and adrenoreceptor beta 2 (ADRB2, as shown in SEQ ID NO:5). In some embodiments, the expression of all of these genes may be modulated in these methods.

In some embodiments, the method comprises contacting a cell with a combination of THC and CBD.

In some embodiments, contacting the cell with the combination of THC and CBD may be achieved by administering to a subject a combination of THC and CBD.

The combination of THC and CBD may be provided in the form of any of the liquid compositions described herein. The liquid compositions may be provided to the cell and/or administered to the subject in any of the dosages described herein.

Chemokine (C-C motif) ligand 5 gene (CCL5) encodes a chemokine and functions as a chemoattractant for blood monocytes, memory T helper cells and eosinophils. It causes the release of histamine from basophils and activates eosinophils. Among its related pathways are pigment epithelium-derived factor (PEDF) induced signaling and toll like receptor signaling pathway. Therefore, an upregulation of this gene may result in improved immune cell communication, organization and coordination.

Cannabinoid Receptor 2 gene (CNR2) encodes the CB2 receptor also known as the peripheral cannabinoid receptor. The CB2 receptor is located primarily in immune cells both within and outside of the CNS. The upregulation of this gene may reinforce the physiological uptake involved in metabolism of the treated active compounds at a cellular level.

Interleukin 8 gene (CXCL8) encodes interleukin 8 (IL-8), which is a protein that is a member of the CXC chemokine family and is a major mediator of the inflammatory response. The encoded protein is secreted primarily by neutrophils, where it serves as a chemotactic factor by guiding the neutrophils to the site of infection. This chemokine is also a potent angiogenic factor. Among its related pathways are PEDF Induced Signaling. IL-8 is a chemotactic factor that attracts neutrophils, basophils, and T-cells, but not monocytes. It is also involved in neutrophil activation. It is released from several cell types in response to an inflammatory stimulus. It is a potent proinflammatory cytokine and implicated in various pain states including cancer pain. A downregulation of this gene suggests an anti-inflammatory effect.

The cerebellar degeneration-related protein 2 gene (CDR2) encodes a protein that plays a role in cellular calcium transport and nerve function at the mitochondrial level. CDR2 is linked with CB1 receptor activation and is believed to form part of a pathway for the neuroprotective role attributed to CBD.

The adrenoreceptor beta 2 gene (ADRB2) encodes the beta-2 adrenergic receptor. Adrenaline is a ligand of this receptor. ADRB2 has been linked to the pain response, where antidepressants have been reported as suppressing neuropathic pain mediated by a peripheral B-adrenoreceptor mediated anti-tumour necrosis factor-α (TNFα) mechanism. Actions of ADRB2 are related to facilitation of the fight-or-flight response but also are involved in neuroinflammatory processes. ADRB2 stimulation is also thought to increase migration of hematopoietic progenitor cells and stem cells.

The expression levels of a gene may be determined according to any technique known in the art.

In some embodiments, CNR2 is upregulated following administration of the combination of THC and CBD. The upregulation may be at least about 0.5-fold, 1-fold, 1.5-fold, 2-fold, 2.5-fold or 3-fold increase over expression levels of the gene in a sample obtained prior to exposure to the combination of THC and CBD. The upregulation may persist for at least about 1, 1.5, 6, 12, 18, 24, 48, 36, 72 hours or longer after THC and CBD exposure.

In some embodiments, ADRB2 is upregulated following administration of the combination of THC and CBD. The upregulation may be at least about 0.5-fold, 1-fold, 1.5-fold, 2-fold, 2.5-fold or 3-fold increase over expression levels of the gene in a sample obtained prior to exposure to the combination of THC and CBD. The upregulation may persist for at least about 1, 1.5, 6, 12, 18, 24, 48, 36, 72 hours or longer after THC and CBD exposure.

In some embodiments the regulation of a gene (upregulation or downregulation) varies depending on the ratio of THC:CBD exposed to the cell and/or administered to the subject. In some embodiments, exposure to a high-THC composition may result in a downregulation of CCL5. The downregulation may be at least about −0.5-fold or −1-fold change over expression levels of the gene in a sample obtained prior to exposure to THC and CBD. The downregulation may persist for at least about 1, 1.5, 6, 12, 18, 24, 48, 36, 72 hours or longer after THC and CBD exposure. In some embodiments, exposure to a high-CBD composition may result in an upregulation of CCL5. The upregulation may be at least about 0.5-fold, 1-fold, 1.5-fold, 2-fold, 2.5-fold or 3-fold increase over expression levels of the gene in a sample obtained prior to exposure to the combination of THC and CBD. The upregulation may persist for at least about 1, 1.5, 6, 12, 18, 24, 48, 36, 72 hours or longer after THC and CBD exposure.

In some embodiments, exposure to a high-CBD composition may result in a downregulation of CXCL8. The downregulation may be at least about −0.5-fold or −1-fold change over expression levels of the gene in a sample obtained prior to exposure to THC and CBD. The downregulation may persist for at least about 1, 1.5, 6, 12, 18, 24, 48, 36, 72 hours or longer after THC and CBD exposure. In some embodiments, exposure to a high-THC composition may result in an upregulation of CXCL8. The upregulation may be at least about 0.5-fold, 1-fold, 1.5-fold or 2-fold increase over expression levels of the gene in a sample obtained prior to exposure to the combination of THC and CBD. The upregulation may persist for at least about 1, 1.5, 6, 12, 18, 24, 48, 36, 72 hours or longer after THC and CBD exposure.

In some embodiments, exposure to a high-THC composition may result in a downregulation of CDR2. The downregulation may be at least about −0.5-fold, −1-fold, −1.5-fold or −2-fold change over expression levels of the gene in a sample obtained prior to exposure to THC and CBD. The downregulation may persist for at least about 1, 1.5, 6, 12, 18, 24, 48, 36, 72 hours or longer after THC and CBD exposure. In some embodiments, exposure to a high-CBD composition may also result in downregulating and/or upregulating CDR2, for example, within about 70, 48, 36, 24, 12, 6, 5, 4, 3 or 2 hours of THC and CBD exposure, expression of CDR2 may be downregulating, while after about 2, 3, 4, 5, 6, 12, 24, 36, 48 or 70 hours of treatment CDR2 expression may be upregulated. The CDR2 downregulation may be about −0.5-fold, −1-fold or −1.2-fold change over expression levels of the gene in a sample obtained prior to exposure to THC and CBD. The CDR2 upregulation may be about 0.5-fold, 1-fold or 1.2-fold change over expression levels of the gene in a sample obtained prior to exposure to THC and CBD. The downregulation may persist for at least about 1, 1.5, 6, 12, 18, 24, 48, 36, 72 hours or longer after THC and CBD exposure.

In some embodiments, the invention provides a method of downregulating CCL5 and CDR2 and upregulating CNR2, CXCL8 and ADRB2, comprising contacting a cell with a combination of THC and CBD, wherein the ratio by weight of THC:CBD is 1 or more:1. The combination may be provided in the form of any of the high THC liquid compositions described herein. The step of contacting the cell with the combination of THC and CBD may comprise administering the combination of THC and CBD to a subject.

In some embodiments, the invention provides a method of downregulating CXCL8 and CDR2 and upregulating CCL5, CNR2 and ADRB2, comprising contacting a cell with a combination of THC and CBD, wherein the ratio by weight of THC:CBD is 1:1.2 or more. The combination may be provided in the form of any of the high-CBD compositions described herein. The step of contacting a cell with the combination of THC and CBD may comprise administering the combination of THC and CBD to a subject.

In some embodiments, the invention provides a method of downregulating CXCL8 and upregulating CCL5, CNR2 and ADRB2, comprising contacting a cell with a combination of THC and CBD, wherein the ratio by weight of THC:CBD is 1:1.2 or more. The combination may be provided in the form of any of the high-CBD compositions described herein. The step of contacting a cell with the combination of THC and CBD may comprise administering the combination of THC and CBD to a subject.

In some embodiments, the method of gene regulation is also a method for treating pain, inflammation and/or anxiety.

### Biomarkers

One aspect of the invention provides a method of affecting the concentration of a biomarker associated with inflammation. The biomarker may be selected from granulocyte-macrophage colony-stimulating factor (GM-CSF) and interleukin-15 (IL-15) or a combination thereof.

In some embodiments, the method comprises contacting a cell with a combination of THC and CBD. The step of contacting the cell with the combination of THC and CBD may comprise administering the combination of THC and CBD to a subject.

The combination of THC and CBD may be provided in the form of any of the liquid compositions described herein.

The concentration of GM-CSF is decreased when a combination of THC and CBD is contacted with the cell and/or administered to the subject. Accordingly, the combination may be provided by any of the liquid compositions described herein. In some embodiments, the maximum peak plasma concentration of GM-CSF following administration of the combination of THC and CBD may be up to about 180 pg/ml, 150 pg/ml, 120 pg/ml, 100 pg/ml, 80 pg/ml, or 70 pg/ml. The minimum peak plasma concentration of GM-CSF CSF following administration of the combination of THC and CBD may be at least about 20 pg/ml, 30 pg/ml or 40 pg/ml. The peak plasma concentration of GM-CSF CSF following administration of the combination of THC and CBD may be from any of these minimum amounts to any of these maximum amounts, for example, from about 20 pg/ml to about 180 pg/ml or about 40 pg/ml to about 80 pg/ml. The peak concentration of GM-CSF following administration of the combination of THC and CBD may occur within about 7, 6, 5, 4, 3 or 2 hours following administration.

In some embodiments, there is provided a method of decreasing the concentration of interleukin 15, comprising contacting a cell with a combination of THC and CBD, wherein the ratio by weight of THC:CBD is 1 or more:1. The combination may be provided in the form of any of the high THC liquid compositions described herein.

In some embodiments, there is provided a method of decreasing the concentration of interleukin 15, comprising administering to a subject a combination of THC and CBD, wherein the ratio by weight of THC:CBD is 1 or more:1. The combination may be provided in the form of any of the high THC liquid compositions described herein.

In these methods of decreasing IL-15 concentration, the maximum peak plasma concentration of IL-15 following administration of the combination of THC and CBD may be up to about 150 pg/ml or 100 pg/ml. The minimum peak plasma concentration following administration of the combination of THC and CBD of IL-15 may be at least about 20 pg/ml, 40 pg/ml or 50pg/ml. The peak plasma concentration of IL-15 following administration of the combination of THC and CBD may be from any of these minimum amounts to any of these maximum amounts, for example, from about 20 pg/ml to about 150 pg/ml. The peak plasma concentration of IL-15 following administration of the combination of THC and CBD may be reached within about 4.5 hours following administration.

In some embodiments, there is provided a method of increasing the concentration of interleukin 15, comprising contacting a cell with a combination of THC and CBD, wherein the ratio by weight of THC:CBD is 1:1.2 or more. The combination may be provided in the form of any of the high-CBD compositions described herein.

In some embodiments, there is provided a method of increasing the concentration of interleukin 15, comprising administering to a subject a combination of THC and CBD, wherein the ratio by weight of THC:CBD is 1 or more:1. The combination may be provided in the form of any of the high-CBD compositions described herein.

In these methods of increasing IL-15 concentration, the maximum peak plasma concentration of IL-15 following administration of the combination of THC and CBD may be up to about 1500 pg/ml, 1000 pg/ml, 750 pg/ml, 650 pg/ml or 600 pg/ml. The minimum peak plasma concentration following administration of the combination of THC and CBD of IL-15 may be at least about 250 pg/ml, 300 pg/ml, 350 pg/ml, 400 pg/ml, 450 pg/ml or 500 pg/ml. The peak plasma concentration of IL-15 following administration of the combination of THC and CBD may be from any of these minimum amounts to any of these maximum amounts, for example, from about 250 pg/ml to about 1500 pg/ml. The peak plasma concentration of IL-15 following administration of the combination of THC and CBD may be reached within about 4.5, 4 or 3.5 hours following administration. By way of comparison, peak plasma concentration of IL-15 following administration of vehicle only is about 220 pg/ml achieved at about 5 hours after administration.

Some embodiments of the methods of treatment described herein further comprise affecting the concentration of GM-CSF and/or IL-15.

The concentration of a biomarker in a sample taken from a subject may be determined according to any technique known in the art.

### EXAMPLES

The invention will be further described by way of non-limiting example(s). It will be understood to persons skilled in the art of the invention that many modifications may be made without departing from the spirit and scope of the invention.

### Example 1—Administration of Cannabinoid Containing Compositions to Dogs

This Example describes single oral dosing to dogs of two compositions comprising different ratios of THC and CBD. After administration, blood was taken from the animals and they were observed for evidence of adverse effects or toxicity.

Two compositions, IVP1 and IVP2, were administered in varying amounts in the study. The IVP's can be administered separately as in the case of the present study or they can be mixed together to provide a fixed ratio of THC and CBD as described in Examples 2 and 3.

Investigational Veterinary Product 1 (IVP1):
Composition—4.75 mg/ml THC in MCT oil; Formulation type-Oil-based solution

Investigational Veterinary Product 2: (IVP2):
Composition—4.58 mg/ml CBD in MCT oil; Formulation type-Oil-based solution

Control Veterinary Product (CVP):
Composition-Medium Chain Triglyceride (MCT) oil no active ingredients; Formulation type—Carrier oil

### Design

Eleven (11) dogs received single concomitant oral doses of cannabidiol (CBD) and tetrahydrocannabinol (THC) oils, or placebo.
Three (3) dogs served as controls and received MCT oil with no active ingredients (Treatment 1).
Treatment 2 were dosed with IVP1 and IVP2 in a 2:1 ratio which was administered to four (4) dogs.
Treatment 3 were dosed with IVP1 and IVP2 in a 1:2 ratio which was administered to four (4) dogs.

### Dose Calculation

Dogs were treated on individual Day −1 bodyweights as indicated in Table 2 below. Dosage volumes were calculated prior to Day 0, to provide the target dosage in mg/kg bodyweight based on actual dog bodyweights and IVP active ingredient composition based on the Certificates of Analysis.

TABLE 2
Treatment Regime

| Treatment | | | | Treatment | Number of |
|---|---|---|---|---|---|
| Group | Dose Rate | Route | Frequency | Day | Animals |
| 1 | MCT oil | Oral | Once | 0 | 3 (Dogs 9-11) |
| | 0.1 mL/kg | | | | |
| 2 | 0.24 mg/kg | Oral | Once | 0 | 4 (Dogs 1-4) |
| | THC and | | | | |
| | 0.12 mg/kg | | | | |
| | CBD | | | | |
| 3 | 0.12 mg/kg | Oral | Once | 0 | 4 (Dogs 4-8) |
| | THC and | | | | |
| | 0.24 mg/kg | | | | |
| | CBD | | | | |

### Monitoring and Recording Adverse Events

Each study animal was closely monitored throughout the study period for the development of the following clinical signs (which have been associated with THC toxicity): sedation/lethargy, agitation/involuntary movement, locomotion issues, eye changes, coughing, drooling/salivating, gagging/retching, vomiting, diarrhea, incontinence, heart rates, respiratory rates, temperature changes, and/or any other abnormal observation.

A serious adverse event (SAE) was defined as an AE which is fatal or life-threatening, or results in significant disability or incapacity or a congenital anomaly/birth defect or results in permanent or prolonged signs which require professional intervention beyond routine prevention measures and common first aid. An AE was automatically classified as serious if one of the following reactions was reported: shock (all shock reactions whatever the origin is: circulatory, anaphylactic), collapse, syncope, immune mediated disease (for adverse reaction), seizures/convulsions, blindness, deafness, paralysis/paresis, malignant neoplasia (including sarcomas at the administration site), respiratory distress, dyspnoea, apnoea, failure of vital organs (total loss of function), uterine haemorrhage, uterine rupture, or pyometra, peritonitis, diabetes mellitus (if reports as SAR), and/or injection site reactions with systemic involvement and reduced mobility.

### Blood Sampling

Blood samples of approximately 5 mL were collected at each drug concentration sampling time-point from each animal using 22-gauge needles and 5 mL syringes.

### Results

TABLE 3
Actual Dosages Administered

| | Day −1 | Dosage | CBD | Treatment |
|---|---|---|---|---|
| Dog No. | weight (kg) | THC (mg) | dosage (mg) | Group |
| Dog 1 | 17.6 | 4.22 | 2.11 | 2 |
| Dog 2 | 17.6 | 4.22 | 2.11 | 2 |
| Dog 3 | 20.7 | 4.97 | 2.48 | 2 |
| Dog 4 | 18.2 | 4.37 | 2.18 | 2 |
| Dog 5 | 21.2 | 2.54 | 5.01 | 3 |
| Dog 6 | 18.6 | 2.23 | 4.46 | 3 |
| Dog 7 | 16.1 | 1.93 | 3.86 | 3 |
| Dog 8 | 18.5 | 2.22 | 4.44 | 3 |
| Dog 9 | 17.7 | 0 | 0 | 1 |
| Dog 10 | 21.1 | 0 | 0 | 1 |
| Dog 11 | 20.7 | 0 | 0 | 1 |

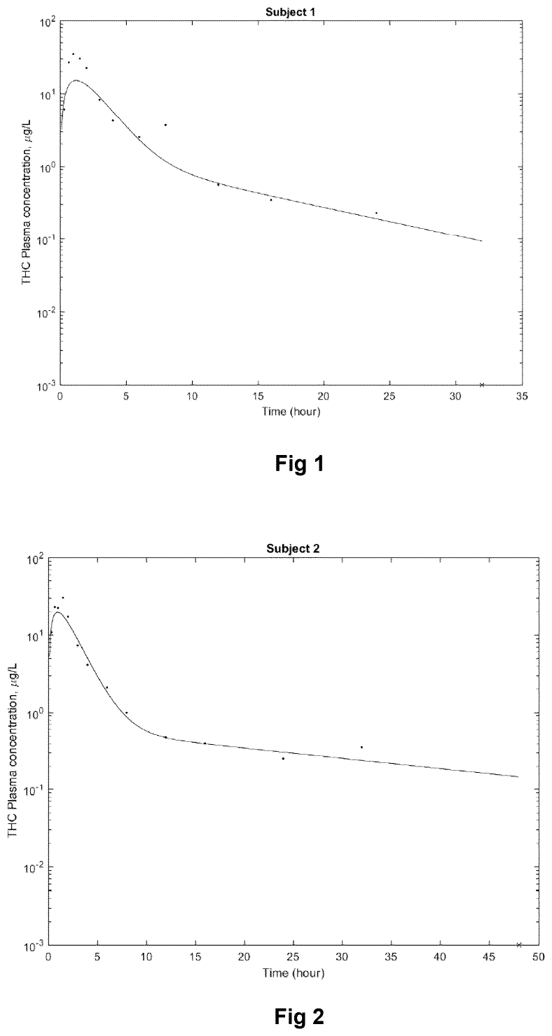

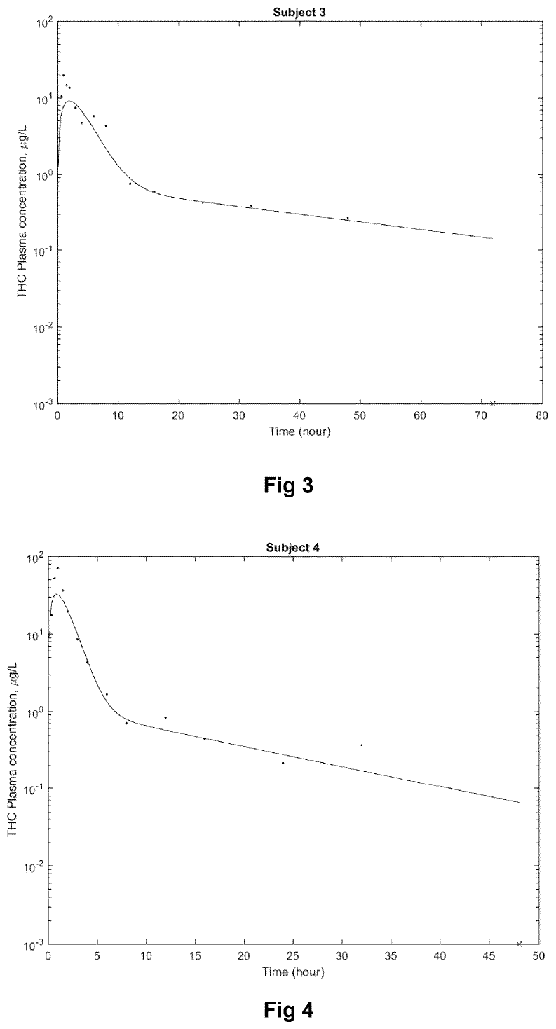

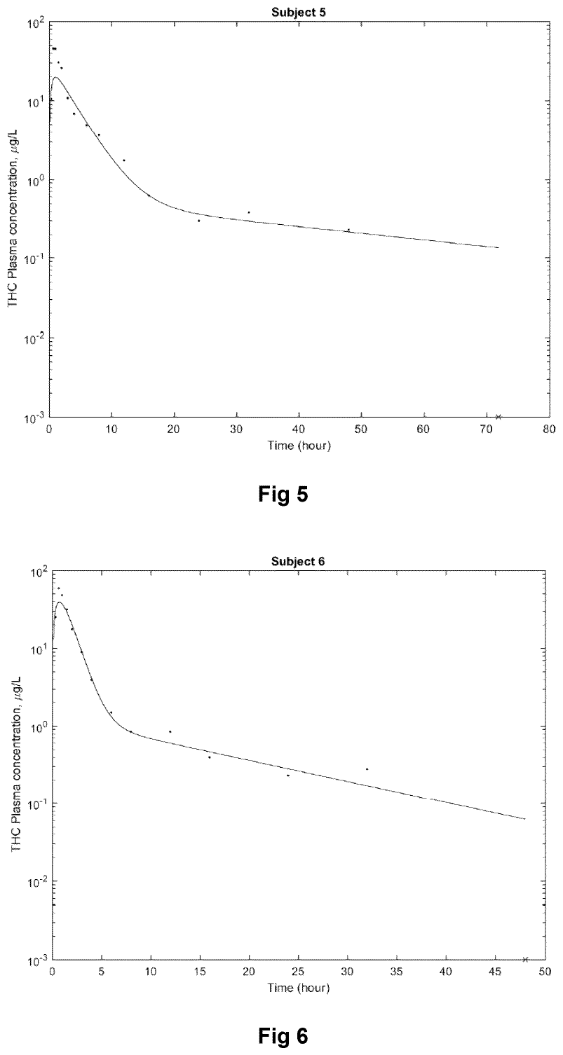

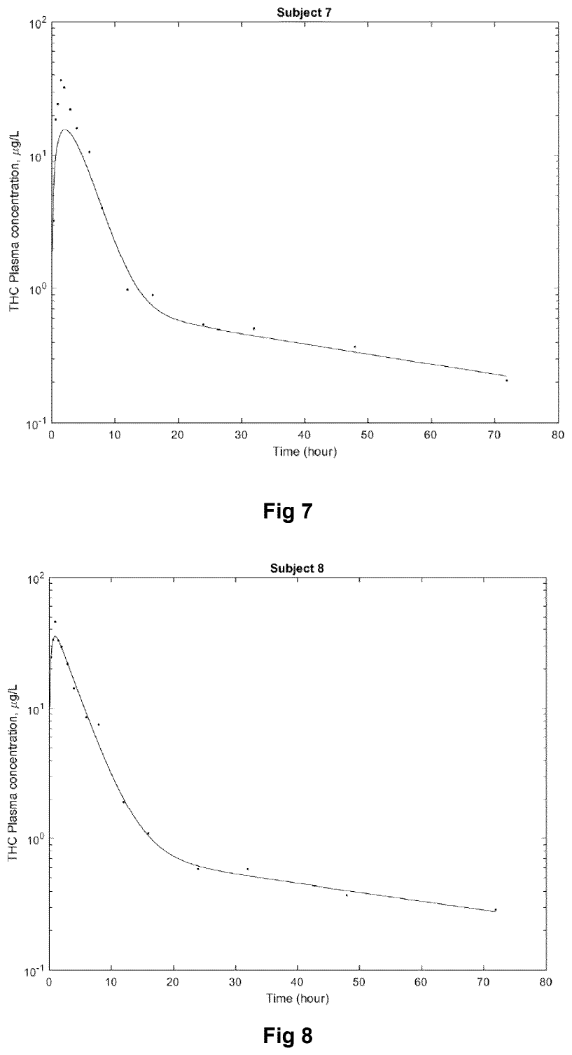

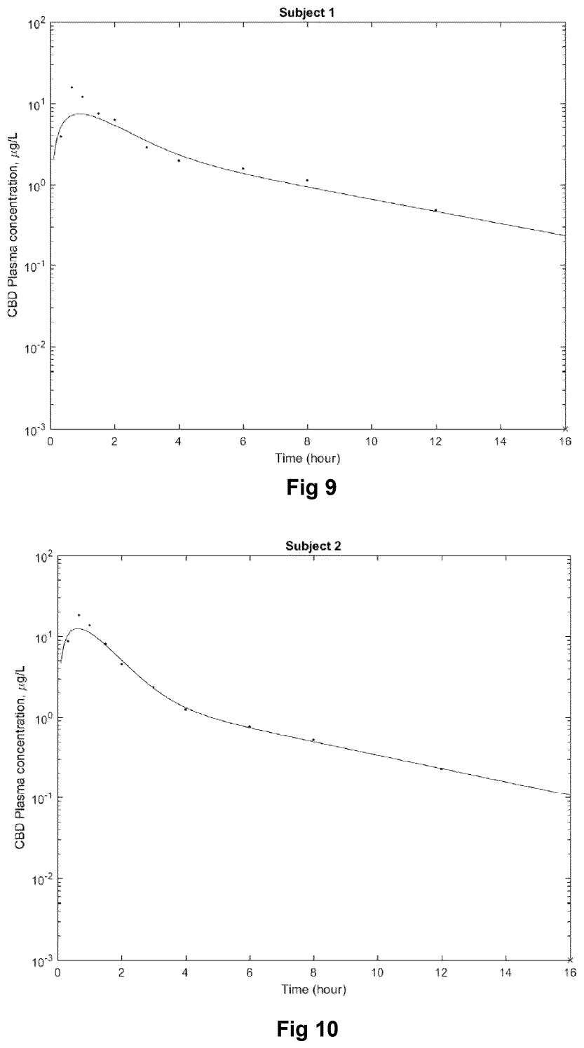

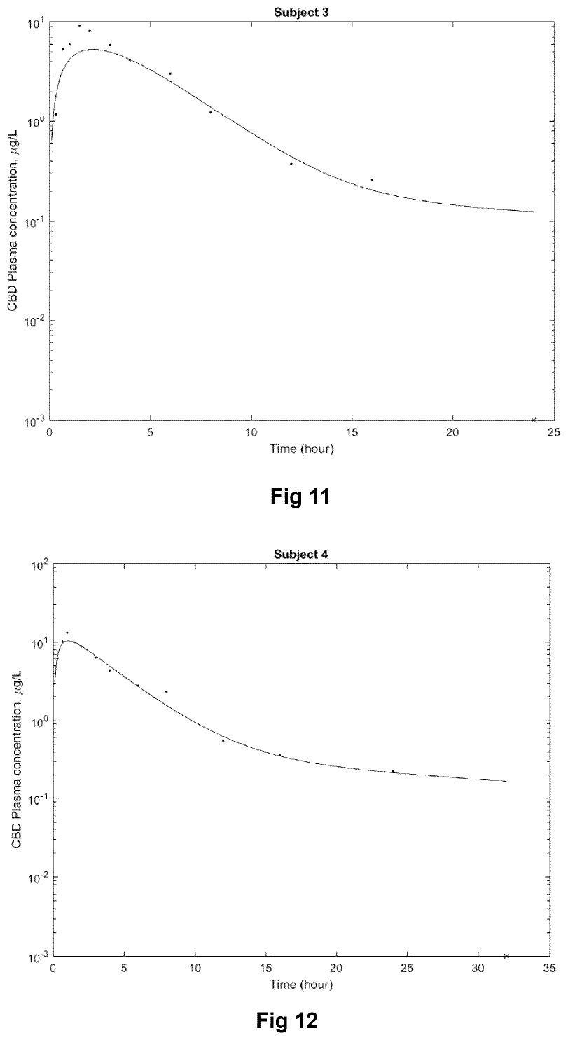

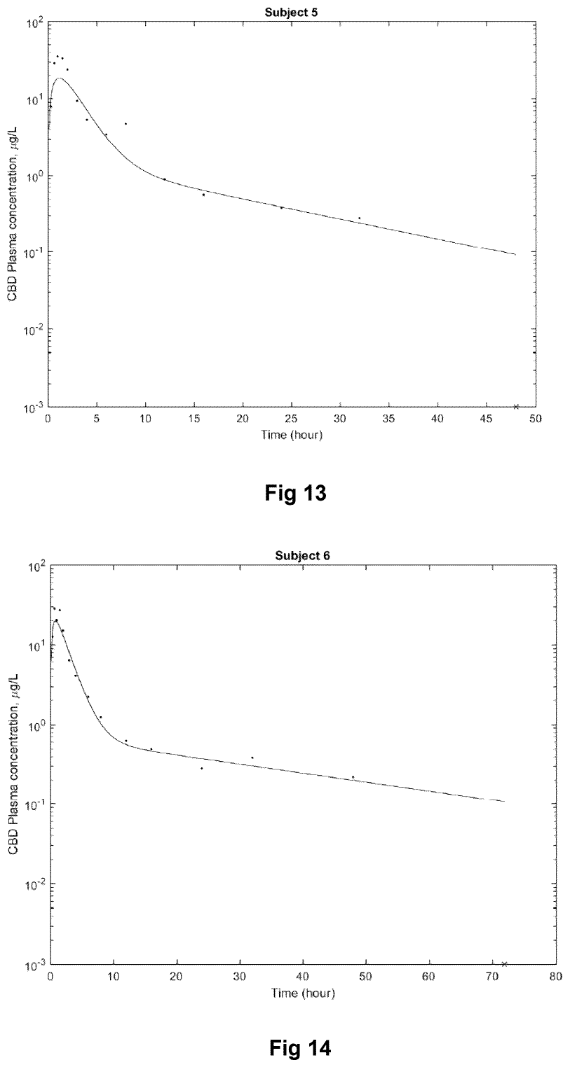

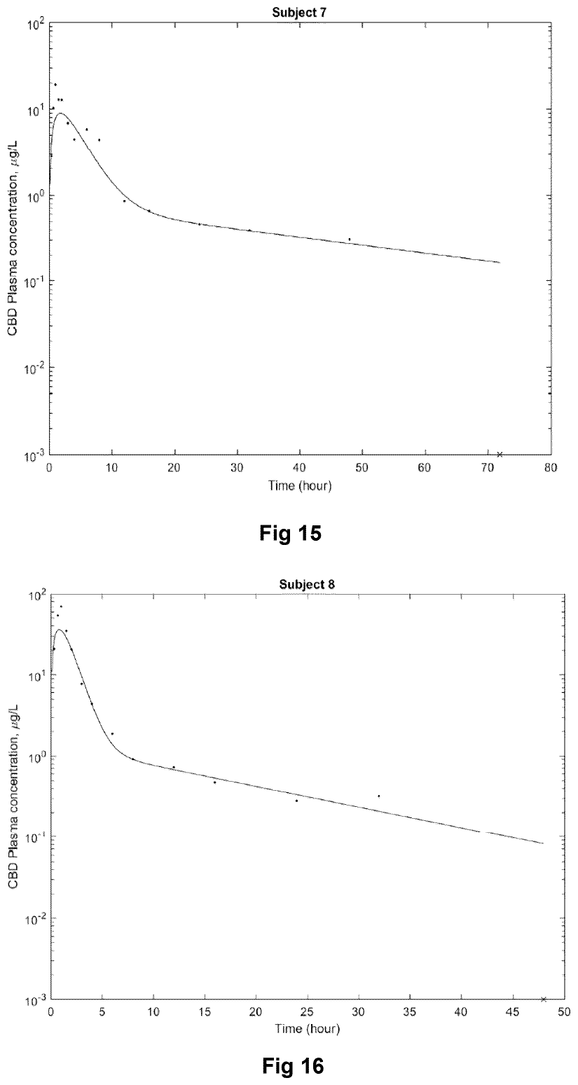

The results of the blood sampling are set out for CBD in Table 4 and for THC in Table 5. The data points from Table 4 and Table 5 were also plotted and these graphs are set out in FIGS. 1-16.

TABLE 4
Results of CBD administration and sampling

| Day −1 | 17.6 | 17.6 | 20.7 | 18.2 | | 21.2 | 18.6 | 16.1 | 18.5 | |
|---|---|---|---|---|---|---|---|---|---|---|
| weight (kg) | | | | | | | | | | |
| CBD dose | 2.11 | 2.11 | 2.48 | 2.18 | | 5.01 | 4.46 | 3.86 | 4.44 | |
| (mg) | | | | | | | | | | |
| CBD (mg/kg) | 0.12 | 0.12 | 0.12 | 0.12 | | 0.24 | 0.24 | 0.24 | 0.24 | |
| | | | | | Mean | | | | | Mean |
| Dog ID | 1 | 2 | 3 | 4 | 0.12 mg/kg | 5 | 6 | 7 | 8 | 0.24 mg/kg |
| 0 | 0 | 0 | 0 | 0 | 0.00 | 0 | 0 | 0 | 0 | 0.00 |
| 0.33 | 3.83 | 8.63 | 1.18 | 6.08 | 4.93 | 7.79 | 12.50 | 2.79 | 20.60 | 10.92 |
| 0.67 | 15.60 | 18.10 | 5.26 | 10.10 | 12.27 | 28.70 | 28.10 | 10.20 | 53.40 | 30.10 |
| 1 | 12.00 | 13.60 | 5.99 | 13.10 | 11.17 | 35.20 | 20.10 | 18.90 | 69.10 | 35.83 |
| 1.5 | 7.50 | 7.99 | 9.07 | 9.84 | 8.60 | 33.20 | 27.00 | 12.70 | 34.60 | 26.88 |
| 2 | 6.27 | 4.48 | 8.08 | 8.72 | 6.89 | 23.70 | 14.90 | 12.60 | 20.30 | 17.88 |
| 3 | 2.83 | 2.33 | 5.83 | 6.25 | 4.31 | 9.21 | 6.33 | 6.79 | 7.66 | 7.50 |
| 4 | 1.95 | 1.24 | 4.08 | 4.31 | 2.90 | 5.28 | 4.07 | 4.40 | 4.36 | 4.53 |
| 6 | 1.56 | 0.76 | 2.99 | 2.75 | 2.02 | 3.40 | 2.22 | 5.74 | 1.87 | 3.31 |
| 8 | 1.12 | 0.53 | 1.23 | 2.32 | 1.30 | 4.68 | 1.23 | 4.36 | 0.91 | 2.79 |
| 12 | 0.48 | 0.23 | 0.37 | 0.55 | 0.41 | 0.88 | 0.63 | 0.85 | 0.72 | 0.77 |
| 16 | 0.00 | 0.00 | 0.26 | 0.36 | 0.16 | 0.55 | 0.49 | 0.65 | 0.47 | 0.54 |
| 24 | | | 0.00 | 0.23 | 0.11 | 0.38 | 0.28 | 0.46 | 0.28 | 0.35 |
| 32 | | | | 0.00 | 0.00 | 0.28 | 0.39 | 0.39 | 0.32 | 0.34 |
| 48 | | | | | | 0.00 | 0.22 | 0.31 | 0.00 | 0.13 |
| 72 | | | | | | | 0.00 | 0.00 | | 0.00 |

The results of the CBD sampling provided in Table 4 demonstrate a good uptake of CBD by the subject canines. First peaks or Cₘₐₓ were achieved between 0.67 and 1.5 hours in all subjects. First peak concentrations of CBD ranged from 5 ng/mL to 69 ng/ml. The lowest level of CBD recorded in a subject 12 hours post administration was 0.23 ng/ml CBD. The highest level recorded 12 hours post administration was 0.88 ng/ml. CBD concentrations of 0.26 ng/ml and 0.28 ng/ml were recorded in subjects 16 and 32 hours post administration. The calculated AUC_{0-∞} is 0.339 ng·hour/mL and the terminal half-life ((t½) of 12.6 hours.

TABLE 5
Results of THC administration and sampling

| Day −1 | 21.2 | 18.6 | 16.1 | 18.5 | | 17.6 | 17.6 | 20.7 | 18.2 | |
|---|---|---|---|---|---|---|---|---|---|---|
| weight (kg) | | | | | | | | | | |
| THC (mg/kg) | 2.54 | 2.23 | 1.93 | 2.22 | | 4.22 | 4.22 | 4.97 | 4.37 | |
| CBD (mg/kg) | 0.12 | 0.12 | 0.12 | 0.12 | | 0.24 | 0.24 | 0.24 | 0.24 | |
| | | | | | Mean | | | | | Mean |
| Dog ID | 5 | 6 | 7 | 8 | 0.12 mg/kg | 1 | 2 | 3 | 4 | 0.24 mg/kg |
| 0 | 0 | 0 | 0 | 0 | 0.00 | 0 | 0 | 0 | 0 | 0.00 |
| 0.33 | 6.03 | 10.70 | 2.65 | 17.30 | 9.17 | 10.40 | 24.80 | 3.22 | 24.40 | 15.71 |
| 0.67 | 26.60 | 22.80 | 10.50 | 51.50 | 27.85 | 45.40 | 59.00 | 18.50 | 33.00 | 38.98 |
| 1 | 34.30 | 22.10 | 19.60 | 71.30 | 36.83 | 45.20 | 47.80 | 24.20 | 45.60 | 40.70 |
| 1.5 | 30.00 | 30.00 | 14.60 | 36.20 | 27.70 | 30.10 | 31.40 | 36.40 | 32.70 | 32.65 |
| 2 | 22.30 | 17.10 | 13.50 | 19.20 | 18.03 | 25.60 | 17.40 | 32.20 | 29.40 | 26.15 |
| 3 | 8.19 | 7.28 | 7.41 | 8.47 | 7.84 | 10.70 | 8.85 | 22.10 | 21.60 | 15.81 |
| 4 | 4.19 | 4.09 | 4.62 | 4.25 | 4.29 | 6.77 | 3.88 | 15.80 | 14.10 | 10.14 |
| 6 | 2.50 | 2.09 | 5.75 | 1.64 | 3.00 | 4.86 | 1.48 | 10.50 | 8.47 | 6.33 |
| 8 | 3.64 | 0.99 | 4.21 | 0.70 | 2.38 | 3.61 | 0.84 | 4.01 | 7.46 | 3.98 |
| 12 | 0.56 | 0.47 | 0.75 | 0.83 | 0.65 | 1.72 | 0.84 | 0.97 | 1.91 | 1.36 |
| 16 | 0.34 | 0.40 | 0.60 | 0.44 | 0.44 | 0.62 | 0.39 | 0.88 | 1.09 | 0.75 |
| 24 | 0.23 | 0.25 | 0.42 | 0.22 | 0.28 | 0.30 | 0.23 | 0.53 | 0.58 | 0.41 |
| 32 | 0.00 | 0.35 | 0.39 | 0.36 | 0.28 | 0.38 | 0.28 | 0.50 | 0.58 | 0.43 |
| 48 | | 0.00 | 0.27 | 0.001 | 0.09 | 0.23 | 0.001 | 0.37 | 0.37 | 0.24 |
| 72 | | | 0.00 | | 0.00 | 0.00 | | 0.21 | 0.29 | 0.16 |

THC levels recorded in subject's blood plasma fraction are set out in Table 5. First peaks or Cₘₐₓ were achieved between 0.67 and 1.5 hours in all subjects. First peak concentrations of CBD ranged from 19 ng/ml to 71.3 ng/ml. The lowest level of CBD recorded in a subject 12 hours post administration was 0.67 ng/mL CBD. The highest level recorded 12 hours post administration was 1.91 ng/ml. CBD concentrations of 0.34 ng/ml and 0.28 ng/ml were recorded in subject's 16 and 32 hours post administration. The calculated AUC_{0-∞} is 0.6 ng·hour/mL and the half-life (t½) of 18.4 hours.

TABLE 6
Clinical Observations

| | | | 1 hr | 1 hr | 2 hr | 3 hr | |
|---|---|---|---|---|---|---|---|
| Group | Dog | 42 min | 10 min | 28 min | 28 min | 10 min | |
| 1 | 9 | Alert, sitting | Alert, standing | Normal | Alert, sitting | Sitting, alert | Dog remained |
| | | | | | | | alert for duration |
| 1 | 10 | Active | Alert, standing | Normal | Alert, sitting, | Being bled | Dog remained |
| | | | | | jumping | | alert for duration |
| 1 | 11 | Alert | Alert | Normal | Alert | Alert | Dog remained |
| | | | | | | | alert for duration |
| 2 | 2 | Alert, standing | Head bobbing, | Looked sleepy | Asleep | Alert, sitting | Some evidence of |
| | | | sitting up | | | | relaxation after 1 hr |
| 2 | 1 | Sitting, quiet | Alert, standing | Normal | A bit sleepy, | Dozing, sitting | Some evidence of |
| | | | | | sitting | | relaxation after 2.5 hr |
| 2 | 4 | Quiet, asleep | Alert, standing | Sleepy | Asleep | Alert | Some evidence of |
| | | | | | | | relaxation from 40 min |
| 2 | 3 | Quiet | Alert, sitting, | Licking self, | A bit sleepy, | Dozing | Some evidence of |
| | | | chewing feet | a bit sleepy | licking | | relaxation after 1.5 hr |
| 3 | 7 | Standing | Alert, sitting | Sleeping | Alert | Sitting, dozing | Some evidence of |
| | | | | | | | relaxation after 1.5 hr |
| 3 | 5 | Alert, sitting, | Sitting/lying, | Lying down | A bit sleepy | Asleep | Some evidence of |
| | | relaxed | alert | | | | relaxation after 2.5 hr |
| 3 | 6 | Sitting | Alert, standing | A littlesleepy | A bit sleepy, | Alert | Some evidence of |
| | | | | | sitting | | relaxation after 1.5 hr |
| 3 | 8 | Quiet | Alert, sitting | Normal | — | Dozing | Some evidence of |
| | | (was before) | | | | | relaxation from 3 hr |

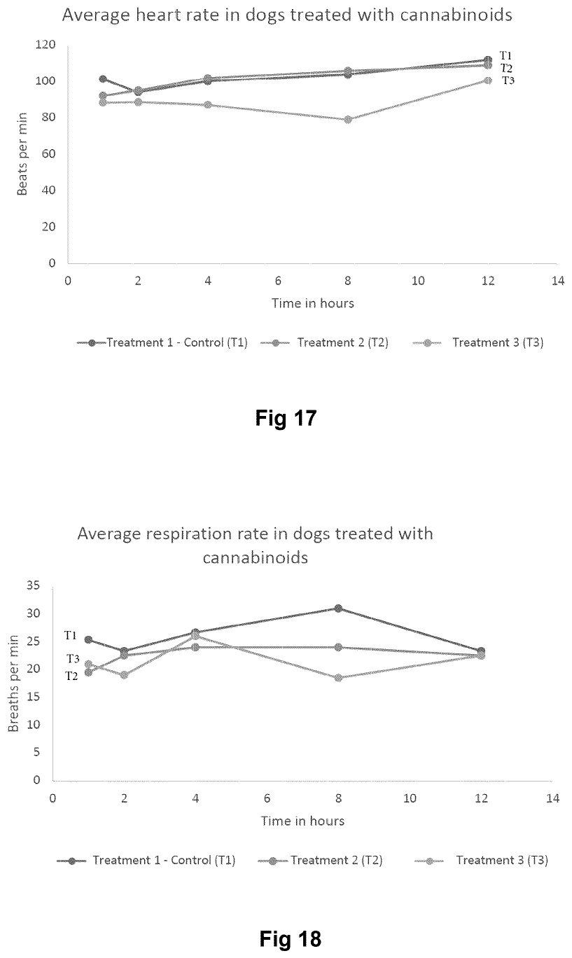

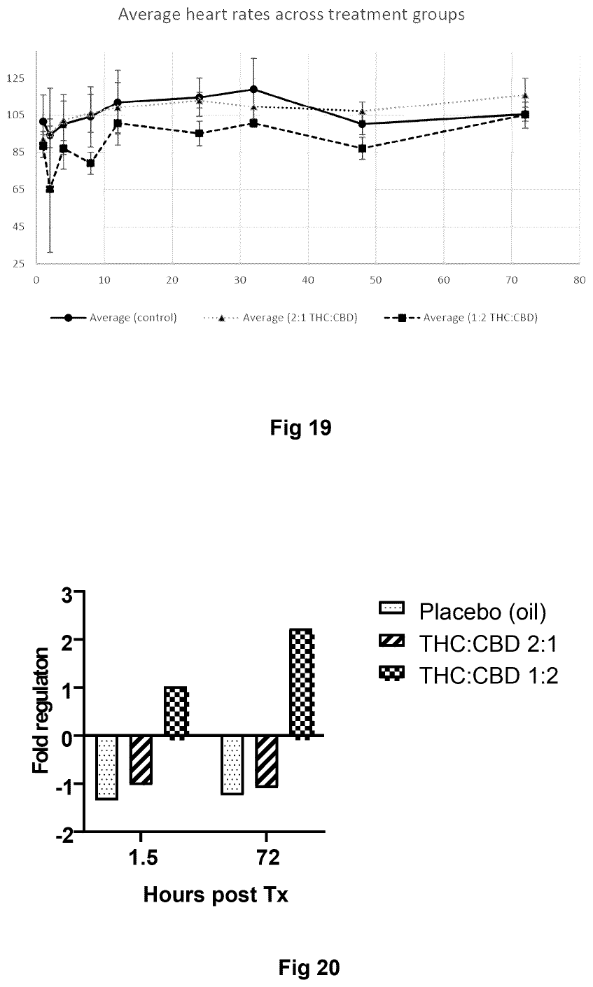

Canine subjects were observed for 12 hours post administration. The results of the visual observations are recorded in Table 6. The physical heart rates and respiratory rates recorded for each subject have been included in Table 7 and the averages for each treatment group were plotted against each other in FIGS. 17 and 18 for heart rate and respiratory rate respectively.

It should be noted that the throughout the 12 hours of observation there was no evidence of any adverse effect arising as a result of the administration of CBD/THC. There were two dogs that exhibited red eyes however two of the controls (TG3) also exhibited the same sign and so redness of the eyes was not attributed to the IVP administration. Importantly, no locomotive issues were seen in the second and third treatments groups that received CBD and THC in the administration. Whilst there were some evidence of relaxation or mild sedation the dogs did not seem incapacitated in any way and did not struggle to walk when they got up to move. There were no gastro-intestinal effects observed, such as diarrhea or vomiting and all the animals were well and ate normally the next day post administration.

TABLE 7
Heart rate and Respiratory Rate data

| Treatment | Hours Post Treatment | | | | | | | | | |
|---|---|---|---|---|---|---|---|---|---|---|
| Group | | 1 | 2 | 4 | 8 | 12 | 24 | 32 | 48 | 72 |
| 1 | HR | 100 | 72 | 80 | 104 | 120 | 120 | 125 | 92 | 100 |
| | RR | 24 | 16 | 20 | 26 | 264 | 20 | 20 | 24 | 28 |
| 2 | HR | 96 | 104 | 120 | 124 | 132 | 120 | 100 | 104 | 112 |
| | RR | 16 | 30 | 24 | sniffing | 32 | 32 | 26 | | 24 |
| 2 | HR | 88 | 92 | 96 | 100 | 104 | 108 | 104 | 100 | 104 |
| | RR | 18 | 24 | 20 | 22 | 18 | 20 | 22 | 32 | 20 |
| 2 | HR | 88 | 100 | 92 | 100 | 104 | 112 | 126 | 112 | 120 |
| | RR | 24 | 18 | 24 | 24 | 20 | panting | 28 | 16 | 24 |
| 1 | HR | 84 | 80 | 100 | 84 | 88 | 100 | 96 | 104 | 108 |
| | RR | 16 | 16 | 24 | sniffing | 16 | panting | 22 | 24 | panting |
| 1 | HR | 120 | 130 | 120 | 124 | 128 | 124 | 136 | 104 | 108 |
| | RR | 36 | 38 | 36 | 36 | 28 | panting | 28 | 24 | 28 |
| 3 | HR | 96 | 10 | 76 | 80 | 92 | 100 | 98 | 80 | 112 |
| | RR | 16 | 18 | 24 | 16 | 22 | 20 | 22 | 20 | 28 |
| 3 | HR | 92 | 66 | 100 | 88 | 104 | 100 | 100 | 88 | 112 |
| | RR | 18 | 18 | 28 | 20 | 22 | 24 | 24 | 20 | 24 |
| 3 | HR | 84 | 84 | 76 | 72 | 88 | 84 | 104 | 84 | 100 |
| | RR | 18 | 16 | 24 | 18 | 18 | panting | 26 | 20 | 24 |
| 2 | HR | 96 | 84 | 100 | 100 | 96 | 112 | 108 | 112 | 128 |
| | RR | 20 | 18 | 28 | 26 | 20 | 24 | 22 | 20 | 20 |
| 3 | HR | 81 | 100 | 96 | 76 | 118 | 96 | 100 | 96 | 96 |
| | RR | 32 | 24 | 28 | 20 | 28 | 28 | 28 | 20 | 24 |

The heart rate data plotted in FIG. 17 shows that there was little difference between the control group (TG1) and (TG2) but there did seem to be a difference with TG3 which received the lower THC dose than then CBD dose. In the respiratory rate graph in FIG. 18, TG3 is also shown as being somewhat lower levels to both TG1 (control) and TG2 (increased CBD). This shows that the concentrations of cannabinoids seen in the study are having physiological effects, up to 12 hours after administration.

### Example 2

The composition comprises a combination of THC and CBD in a carrier oil. The THC and CBD were prepared into separate stock solutions in MCT oil at known concentrations and then a portion of each stock solution was mixed to a pre-determined desired ratio of 2:1 THC to CBD. This ratio of cannabinoids is in a MCT oil. Three compositions were prepared comprising a combined concentration of THC and CBD of 0.9% (i.e. about 0.6% THC and about 0.3% CBD), 1.9% and 2.7%.

### Example 3

The composition comprises a combination of THC and CBD in a carrier oil. The THC and CBD were prepared into separate stock solutions in MCT oil at known concentrations and then a portion of each stock solution was mixed to a pre-determined desired ratio of 1:2 THC to CBD. This ratio of cannabinoids is in a MCT oil. Three compositions were prepared comprising a combined concentration of THC and CBD of 0.9% (i.e. about 0.3% THC and about 0.6% CBD), 1.9% and 2.7%.

### Example 4

This Example describes a gene expression analysis undertaken on the blood samples taken during the study described in Example 1. The gene expression analysis was conducted on blood samples taken prior to treatment, 1.5 h after administration and 72 h after administration.

Dog blood drawn and 100 μl blood immediately added to a labelled room temperature RNAprotect Animal Blood Tube which has been closed and gently inverted 8-10 times, then stored upright at 15-25° C. for 2 hours prior to storing in −20° C. freezer and then shipment on dry ice.

RNA extracted from whole blood collected in RNAprotect Animal Blood tubes using RNeasy Protect Animal Blood kit. RNA was eluted in 30 μl into eppendorf tubes and sealed with parafilm.

RNA quality and concentration are determined by using a Nanodrop spectrophotometer to measure the concentration and OD260/280 values of the samples and an Agilent RNA TapeScreen to check RNA quality.

For the integrity measurement, 1 μl of the total RNA was analysed with a RNA ScreenTape using an Agilent TapeStation.

Gene quantification was carried out using real-time reverse transcription polymerase chain reaction (RT-PCR) combined with microarray analysis, using a RT² Profiler PCR Array.

Experimental RNA samples were converted into first strand cDNA. Then, the cDNA templates were mixed with a ready-to-use RT² qPCR Master Mix and aliquoted into each well of the same plate containing pre-dispensed gene specific primer sets. After qPCR relative expression is determined with the ddCt method. Results are shown in FIGS. 20-24.

Complimentary deoxyribonucleic acid (cDNA) synthesis was carried out using a QIAGEN RT² First Strand Kit (Catalog #330401). Or, for samples containing a low RNA yield, the RT² PreAMP reaction was used before qPCR analysis.

### Example 5

This Example describes biomarker analysis undertaken on the blood samples taken during the study described in Example 1. The analysis was conducted on blood samples taken prior to treatment, 1.5 h after administration and 72 h after administration.

Biomarker concentration was determined using a magnetic bead panel comprising magnetic beads loaded with antibodies for each biomarker of interest.

### Example 6

This Example describes a study involving up to about 45 dogs, approximately balanced by sex, of various breeds and ages with osteoarthritis (OA) in any joint(s) resulting in impairment of mobility and/or signs of pain.

Treatment allocation is double-blinded placebo-controlled and randomised. Dogs are diagnosed with OA by a veterinarian. Dogs have been on a reasonably stable treatment regime (treatments listed). Dogs are fed the same recorded type and amount of diet for prior to and during study duration. No other oil-based OA treatments or supportive therapies will be used for 2 weeks before and during the study.

Dogs with OA are divided into four groups. Neither owner nor clinician will be aware of the nature of the supplementation.
1. Treatment group 1 (placebo group—15 dogs) will receive olive oil, twice daily orally at a dose of 1 ml/10 kg twice daily for eight weeks.
2. Treatment group 2 (CPAT01×0.75× group—15 dogs) will receive CannPal AUS; CPAT01 blended oil) orally at a dose of 0.54 mg/kg twice daily on food for eight weeks.
3. Treatment group 3 (CPAT01—1.5× group—15 dogs) will receive CannPal AUS; CPAT01 blended oil) orally at a dose of 1.08 mg/kg twice daily on food for eight weeks.
4. Treatment group 4 (CPAT01—2.5× group—15 dogs) will receive CannPal AUS; CPAT01 blended oil) orally at a dose of 1.8 mg/kg twice daily on food for eight weeks.

Dogs are enrolled with the written consent of owners. All procedures comply with applicable Animal Ethics Committee (AEC) requirements and Australian Animal Care laws and regulations.

At regular intervals (for example at Week −2, 0, 2, 4, 6 and 8) owners filled out a Canine Brief Pain Inventory (CPBI), Canine Orthopaedic Index (COI) and Hudson Activity Scale (or similar) which are validated owner questionnaires to assess how dogs with OA are progressing or deteriorating. Visits to the veterinarian at about Week 0, 4 and 8 include a veterinary subjective evaluation and blood sampling. Blood samples are tested for CBC/biochemistry to monitor hepatic and renal enzymes (urea, creatinine, ALP, ALT) and white blood cells. Plasma cannabinoid (CBD and THC) assays are also performed from blood at each visit.

What is claimed is:

1. A liquid composition comprising a combination of Δ⁹-tetrahydrocannabinol (THC) and cannabidiol (CBD) in a ratio by weight of THC:CBD of from 1:1.5 to 1:30, and an oral delivery system comprising a fatty acid.

2. The liquid composition of claim 1, wherein the ratio by weight of THC:CBD is about 1:about 2.

3. The liquid composition of claim 1, wherein the concentration of THC is up to about 6.5%.

4. The liquid composition of claim 1, wherein the concentration of CBD is up to about 7.5%.

5. The liquid composition of claim 1, wherein the oral delivery system comprises a medium-chain triglyceride (MCT) oil.

6. The liquid composition of claim 1, wherein the liquid composition further comprises an antioxidant.

7. The liquid composition of claim 6, wherein the antioxidant is ascorbic acid, butylated hydroxyanisole, butylated hydroxytoluene (BHT), propyl gallate, a tocopherol, or a combination thereof.

8. A liquid composition comprising a combination of Δ⁹-tetrahydrocannabinol (THC) and cannabidiol (CBD) in a ratio by weight of THC:CBD of from 1.01:1 to 99:1, and an oral delivery system comprising a fatty acid.

9. The liquid composition of claim 8, wherein the ratio by weight of THC:CBD is about 2:about 1.

10. The liquid composition of claim 8, wherein the oral delivery system comprises a medium-chain triglyceride (MCT) oil.

11. The liquid composition of claim 8, wherein the liquid composition further comprises an antioxidant.

12. The liquid composition of claim 11, wherein the antioxidant is ascorbic acid, butylated hydroxyanisole, butylated hydroxytoluene (BHT), propyl gallate, a tocopherol, or a combination thereof.

13. A food product comprising the liquid composition of claim 1.

14. A food product comprising the liquid composition of claim 8.

15. A method of treating pain, inflammation and/or anxiety in a non-human subject in need thereof, comprising orally administering to the non-human subject an effective amount of the liquid composition of claim 1.

16. The method of claim 15, wherein administration of the liquid composition controls a heart rate of the non-human subject.

17. The method of claim 16, wherein the average heart rate of the non-human subject is lowered for at least about 2 h following administration.

18. The method of claim 15, wherein administration of the liquid composition downregulates expression of interleukin 8 gene (CXCL8, as shown in SEQ ID NO:1) and/or upregulates expression of chemokine (C-C motif) ligand 5 gene (CCL5, as shown in SEQ ID NO:4) and/or upregulates expression of cannabinoid Receptor 2 gene (CNR2, as shown in SEQ ID NO:3) and/or upregulates expression of adrenoreceptor beta 2 gene (ADRB2, as shown in SEQ ID NO:5).

19. A method of treating pain, inflammation and/or anxiety in a non-human subject in need thereof, comprising orally administering to the non-human subject an effective amount of the liquid composition of claim 8.

20. The method of claim 19, wherein administration of the liquid composition downregulating expression of chemokine (C-C motif) ligand 5 gene (CCL5, as shown in SEQ ID NO:4) and/or downregulating expression of cerebellar degeneration-related protein 2 gene (CDR2, as shown in SEQ ID NO:2) and/or upregulating expression of cannabinoid Receptor 2 gene (CNR2, as shown in SEQ ID NO:3) and/or upregulating expression of interleukin 8 gene (CXCL8, as shown in SEQ ID NO:1) and/or upregulating expression of adrenoreceptor beta 2 gene (ADRB2, as shown in SEQ ID NO:5).
